(19) United States
(12) Patent Application Publication
(10) Pub. No.: US 2023/0013627 A1
(43) Pub. Date: Jan. 19, 2023

(54) THIAZOLIDINEDIONE ANALOGS FOR THE TREATMENT OF NAFLD AND METABOLIC DISEASES

(71) Applicant: Cirius Therapeutics, Inc., San Diego, CA (US)

(72) Inventors: Gerard R. COLCA, San Diego, CA (US); Howard C. DITTRICH, San Diego, CA (US); Brian K. FARMER, San Diego, CA (US); Gad COTTER, Chapel Hill, NC (US); Beth Anne COTTER-DAVISON, Chapel Hill, NC (US)

(21) Appl. No.: 17/738,974

(22) Filed: May 6, 2022

Related U.S. Application Data
(63) Continuation of application No. PCT/US2020/059548, filed on Nov. 6, 2020.
(60) Provisional application No. 62/933,238, filed on Nov. 8, 2019.
(60) Provisional application No. 63/079,905, filed on Sep. 17, 2020.

(51) Int. Cl. A61K 31/426; A61P 1/16

(52) U.S. Cl. CPC A61K 31/426; A61P 1/16

(57) ABSTRACT

Provided herein are thiazolidinedione analogues that are useful for treating non-alcoholic fatty liver disease (NAFLD), non-alcoholic steatohepatitis (NASH), diabetes, and other metabolic inflammation-mediated disease and disorders. Further, provided herein are non-invasive methods and systems for assessing a subject's risk of having NASH. Moreover, provided herein are non-invasive methods and systems for evaluating whether a treatment of NASH is effective.

### CROSS-REFERENCE

This application is a continuation of International Application No. PCT/US2020/059548, filed Nov. 6, 2020, which claims the benefit of U.S. Provisional Patent Application No. 62/933,238 filed Nov. 8, 2019; and U.S. Provisional Patent Application No. 63/079,905 filed Sep. 17, 2020; each of which is incorporated herein by reference in its entirety.

### BACKGROUND

Peroxisome Proliferator Activated Receptors (PPARs), which are members of the nuclear hormone receptor super family, are ligand-activated transcription factors regulating gene expression. PPARs have been implicated in autoimmune diseases and other diseases (e.g., diabetes mellitus, cardiovascular disease, gastrointestinal disease, and Alzheimer's disease). First generation thiazolidinediones approved for treatment of type II diabetes are direct activators of the PPAR gamma subtype. Newer agents work by modifying the activity of the mitochondrial pyruvate carrier.

The mitochondrial pyruvate carrier (MPC) comprises two proteins, MPC1 and MPC2, that form a carrier complex in the inner mitochondrial membrane. Transport into the mitochondrial matrix is required for pyruvate metabolism and critical for a number of metabolic pathways. Modulation of MPC indirectly affects PPAR networks.

### BRIEF SUMMARY OF THE INVENTION

In an aspect provided herein is a method of treating non-alcoholic fatty liver disease (NAFLD) and/or metabolic syndrome, comprising administering to a subject in need thereof: a therapeutically effective amount of a compound of structural Formula (I):

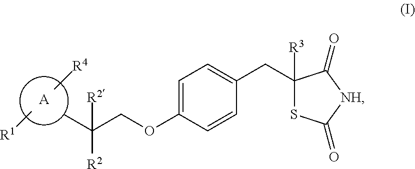

or a pharmaceutically acceptable salt thereof wherein R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some instances, the therapeutically effective amount of the compound comprises a dosage amount of about 62.5 milligrams (mg), about 125 mg, or about 250 mg. In some instances, the compound is administered to the subject once-daily.

In another aspect provided herein is a method of treating at least one metabolic inflammation-mediated disease or disorder, comprising administering to a subject in need thereof a therapeutically effective amount of a compound of structural Formula (I):

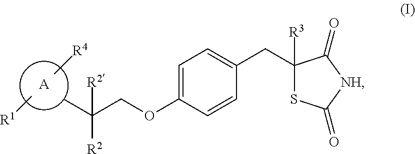

or a pharmaceutically acceptable salt thereof wherein R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some instances, the therapeutically effective amount of the compound comprises a dosage amount of about 62.5 milligrams (mg), about 125 mg, or about 250 mg. In some instances, the compound is administered to the subject once-daily.

In an aspect provided herein is a method of inhibiting cellular (including hepatocyte) mitochondrial pyruvate carrier (MPC), comprising contacting the MPC with a compound as described herein, including embodiments, or the structural Formula (I), or a pharmaceutically acceptable salt thereof. In some instances, the therapeutically effective amount of the compound comprises a dosage amount of about 62.5 milligrams (mg), about 125 mg, or about 250 mg. In some instances, the compound is administered to the subject once-daily.

In an aspect is provided a method of improving or increasing glucose tolerance and/or insulin sensitivity, comprising administering to a subject in need thereof a therapeutically effective amount of a compound as described herein, including embodiments, or the structural Formula (I), or a pharmaceutically acceptable salt thereof. In some instances, the therapeutically effective amount of the compound comprises a dosage amount of about 62.5 milligrams (mg), about 125 mg, or about 250 mg. In some instances, the compound is administered to the subject once-daily.

In an aspect is provided a method of treating or preventing a hepatic disease, disorder, or injury, comprising administering to a subject in need thereof a therapeutically effective amount of a compound as described herein, including embodiments, or the structural Formula (I), or a pharmaceutically acceptable salt thereof. In some instances, the therapeutically effective amount of the compound comprises a dosage amount of about 62.5 milligrams (mg), about 125 mg, or about 250 mg. In some instances, the compound is administered to the subject once-daily.

In one embodiment is a method for treating a patient having disease-promoting polymorphisms in PNPLA3 gene comprising administering to the patient a therapeutically effective amount of a compound of structural Formula (I):

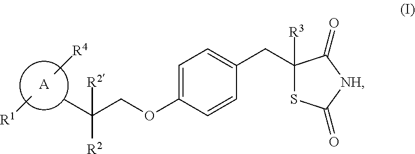

or a pharmaceutically acceptable salt thereof wherein R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some instances, the therapeutically effective amount of the compound comprises a dosage amount of about 62.5 milligrams (mg), about 125 mg, or about 250 mg. In some instances, the compound is administered to the subject once-daily.

In another embodiment, the polymorphism in the PNPLA3 gene confers an increased risk of developing NASH. In another embodiment, the polymorphism is rs738409 single nucleotide polymorphism.

In an aspect is provided a method of treating or preventing liver fibrogenesis, comprising administering to a subject in need thereof a therapeutically effective amount of a compound as described herein, including embodiments, or the structural Formula (I), or a pharmaceutically acceptable salt thereof. In some instances, the therapeutically effective amount of the compound comprises a dosage amount of about 62.5 milligrams (mg), about 125 mg, or about 250 mg. In some instances, the compound is administered to the subject once-daily.

In an aspect provided herein, is a method of treating or preventing nonalcoholic steatohepatitis (NASH) in a subject, comprising administering to a subject in need thereof a therapeutically effective amount of a compound of Formula (I), or a pharmaceutically acceptable salt thereof. In some embodiments, the pharmaceutically acceptable salt thereof is a potassium salt. In some embodiments, the subject is suffering from fibrosis. In some embodiments, the subject is suffering from diabetes. In some embodiments, the diabetes comprises Type II diabetes.

In another aspect provided herein is a method of reducing alanine transaminase (ALT) and/or aspartate aminotransferase (AST) in a subject diagnosed with a nonalcoholic fatty liver disease, the method comprising administering to the subject a therapeutically effective amount of a compound of structural Formula (I):

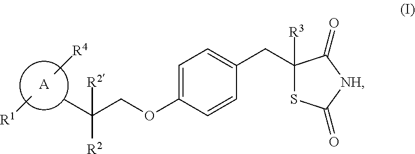

or a pharmaceutically acceptable salt thereof wherein R¹ is independently hydrogen, halogen, substituted or unsubstituted alkyl, or —OR^{1A}, R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some instances, the pharmaceutically acceptable salt comprises a potassium salt. In some instances, the therapeutically effective amount of the compound comprises a dosage amount of about 62.5 milligrams (mg), about 125 mg, or about 250 mg. In some instances, the compound is administered to the subject once-daily.

In another aspect provided herein is a method of reducing hemoglobin A1c (HbA1c) in a subject diagnosed with diabetes, the method comprising administering to the subject a therapeutically effective amount of a compound of structural Formula (I):

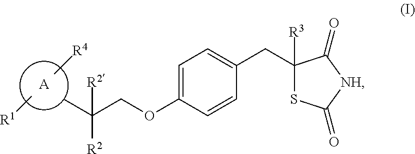

or a pharmaceutically acceptable salt thereof wherein R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some instances, the pharmaceutically acceptable salt comprises a potassium salt.

Aspects disclosed herein provide methods of inhibiting hepatic mitochondrial pyruvate carrier (MPC) with reduced PPARγ agonism, as compared to pioglitazone, in a subject, the method comprising administering to the subject a therapeutically effective amount of a compound of structural Formula (I):

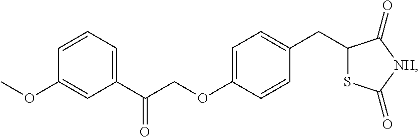 [SMILES: COc1cccc(C(=O)COc2ccc(CC3SC(=O)NC3=O)cc2)c1]

or a pharmaceutically acceptable salt thereof. In some instances, the pharmaceutically acceptable salt thereof comprises a potassium salt. In some instances, the therapeutically effective amount of the compound comprises a dosage amount of about 62.5 milligrams (mg), about 125 mg, or about 250 mg. In some instances, the compound is administered to the subject once-daily.

Disclosed herein, in some embodiments, are pharmaceutical compositions comprising a dosage amount of between about 60 milligrams (mg) and about 250 mg of the compound of structural Formula (I)

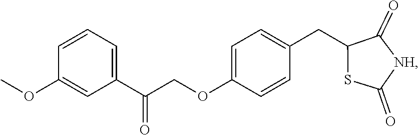 [SMILES: COc1cccc(C(=O)COc2ccc(CC3SC(=O)NC3=O)cc2)c1]

or a pharmaceutically acceptable salt thereof. In some embodiments, the pharmaceutically acceptable salt comprises a potassium salt. In some embodiments, the dosage amount comprises about 62.5 mg. In some embodiments, the dosage amount comprises about 125 mg. In some embodiments, the dosage amount comprises about 250 mg. Also disclosed are methods of treating or preventing a metabolic inflammation-mediated disease or disorder, the method comprising administering the pharmaceutical composition of a compound of Formula (I) to a subject in need thereof. In some embodiments, the subject in need thereof suffers from NASH, Type II diabetes, fibrosis, or a combination thereof. In some embodiments, the subject is prediabetic.

In another aspect, the present disclosure provides a noninvaseive method for analyzing a subject's risk of having non-alcoholic steatohepatitis (NASH). The noninvasive method comprises assaying an Aspartate aminotransferase (AST) level and a hemoglobin A1c (HbA1c) level to generate an output comprising said AST level and HbA1c level indicative that the subject has NASH. The method further comprises comparing said levels of AST and HbA1c to reference levels respectively.

In some embodiments, the reference level of AST is about 27 U/L and the reference level of HbA1c is about 6%. In some embodiments, when said levels of AST and HbA1C are greater than said reference levels, the subject has a risk of having NASH. In some embodiments, the method further comprises subjecting said subject to an image analysis.

In some embodiments, the image analysis comprises ultrasound, CT scan, vibration-controlled transient elastography (VCTE), magnetic resonance imaging (MRI), and magnetic resonance elastography (MRE). In some embodiments, the said output has a specificity of about 95%.

In another aspect, the present disclosure provides a method for evaluating an effectiveness of a treatment for non-alcoholic steatohepatitis (NASH). The method comprises obtaining a baseline level or score of at least three biomarkers selected from the group consisting of Aspartate aminotransferase (AST), Gamma-glutamyltransferase (GGT), Hemoglobin A1c (HbA1c), insulin, Cytokeratin-18 (CK-18), Enhanced Liver Fibrosis (ELF), and alkaline phosphatase in a subject; administering said treatment to said subject for a period of time; obtaining a treatment level or score of said corresponding at least three biomarkers selected from the group consisting of AST, GGT, HbA1c, insulin, CK-18, ELF, and alkaline phosphatase in said subject after said period of time; and generating a composite score based on a difference between said baseline level or score and said treatment level or score, wherein said composite score is indicative of said effectiveness of said treatment.

In some embodiment, the at least three biomarkers are AST, CK-18, and HbA1C. In some embodiment, when said at least three biomarkers are AST, CK-18, and HbA1C, said composite score comprises about 40.3% of a difference of AST between a baseline level or score and a treatment level or score. In some embodiments, wherein when said at least three biomarkers are AST, CK-18, and HbA1C, said composite score comprises about 31.4% of a difference of CK-18 between a baseline level or score and a treatment level or score. In some embodiments, wherein when said at least three biomarkers are AST, CK-18, and HbA1C, said composite score comprises about 28.3% of a difference of HbA1c between a baseline level or score and a treatment level or score.

In some embodiments, the treatment comprises administering pioglitazone to said subject. In some embodiments, the treatment comprises administering Aramachol to said subject. In some embodiments, the treatment comprises administering resmetiron to said subject. In some embodiments, the treatment comprises administering MSDC-0602K to said subject. In some embodiments, the treatment comprises administering liraglutide to said subject.

In some embodiments, the period comprises 6 months. In some embodiments, the period comprises 12 months. In some embodiments, the period comprises 18 months. In some embodiments, the period comprises 24 months.

In some embodiments, the composite score comprises a specificity of at least about 80%. In some embodiments, the composite score comprises a sensitivity of at least about 80%.

In some embodiments, the obtaining a baseline level or score comprises measuring said baseline level or score from a biological sample from said subject. In some embodiments, the obtaining a treatment level or score comprises measuring said treatment level or score from a biological sample from said subject.

In some embodiments, the biological sample comprises whole blood. In some embodiments, the biological sample comprises plasma or serum.

In another aspect, the present disclosure provides a system for evaluating an effectiveness of a treatment for non-alcoholic steatohepatitis (NASH), comprising: a communication interface; and a computer processor operatively coupled to said communication interface, wherein said computer process is programmed to: (i) obtain a baseline level or score of at least three biomarkers selected from the group consisting of Aspartate aminotransferase (AST), Gamma-glutamyltransferase (GGT), Hemoglobin A1c (HbA1c), insulin, Cytokeratin-18 (CK-18), Enhanced Liver Fibrosis (ELF), and alkaline phosphatase in a subject; (ii) administer said treatment to said subject for a period of time; (iii) obtain a treatment level or score of said corresponding at least three biomarkers selected from the group consisting of AST, GGT, HbA1c, insulin, CK-18, ELF, and alkaline phosphatase in said subject after said period of time; and (iv) generate a composite score based on a difference between said baseline level or score and said treatment level or score, wherein said composite score is indicative of said effectiveness of said treatment.

In some embodiments, the subject or patient carries two copies of PNPLA3 rs738409-G. In some embodiments, the subject or patient carries one copy of PNPLA3 rs738409-G and one copy of PNPLA3 rs738409-C. In some embodiments, the subject or patient carries two copies of PNPLA3 rs738409-C.

In another aspect, the present disclosure provides a method for improving a histological feature in a subject's liver, the method comprising administering to the subject a therapeutically effective amount of a compound of structural Formula (I):

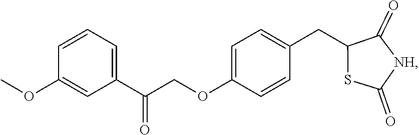 [SMILES: COc1cccc(C(=O)COc2ccc(CC3SC(=O)NC3=O)cc2)c1]

or a pharmaceutically acceptable salt thereof.

In some embodiments, the subject carries two copies of PNPLA3 rs738409-G. In some embodiments, the histological feature is measured by a NAFLD Activity Score (NAS). In some embodiments, the therapeutically effective amount of the compound comprises a dosage amount of about 62.5 milligrams (mg), about 125 mg, or about 250 mg. In some embodiments, the dosage amount is about 125 mg. In some embodiments, the dosage amount is about 250 mg.

### BRIEF DESCRIPTION OF THE DRAWINGS

FIGS. 1A-1C. Show the comparative results from diet-induced obese (60% HF diet) LS-MPC2−/− and WT (fl/fl) mice: Body weight (FIG. 1A); Blood glucose levels (FIG. 1B); Blood glucose AUC (FIG. 1C).

FIGS. 2A-2C. Show the results after a single dose of MSDC-0602 given to LS-MPC2−/− and WT (fl/fl) mice. Blood glucose levels (FIG. 2A); Blood glucose Area Under the Curve (AUC) (FIG. 2B); Plasma insulin levels (FIG. 2C).

FIGS. 3A-3B. Show the results after a single dose of MSDC-0602 given to LS-MPC2−/− and WT (fl/fl) mice. Plasma ALT concentrations (FIG. 3A); Gene expression for markers of liver injury (FIG. 3B).

FIG. 4. A schematic representation of the effects of hepatocyte metabolism on exosome communication with stellate cells.

FIGS. 5A-5I Show the levels of serum miRNAs after treatment of mice with MSDC-0602: mmu-miR-29c-3p (FIG. 5A); mmu-miR-802-3p (FIG. 5B); mmu-miR-802-5p (FIG. 5C); mmu-miR-127-3p (FIG. 5D); mmu-miR-129-2-3p (FIG. 5E); mmu-miR-615-3p (FIG. 5F); mmu-miR-129-5p (FIG. 5G); mmu-miR-205-5p (FIG. 5H); mmu-miR-341-3p (FIG. 5I). (* FDR<0.05 vs LF; † FDR<0.05 vs HTF-C.)

FIG. 6. Is a graphic representation of the data of thyroid hormones with MSDC-0602 in vitro. Line “A” at X=0 is MSDC-0602 itself without T3. Line “B” is T3 doses without MSDC-0602.

FIG. 7. Is a graphic representation of the data of the relative binding affinities of compound MSDC-0602, the metabolite of MSDC-0602, and the two insulin sensitizers rosiglitazone and pioglitazone with PPARγ.

FIG. 8. Is a graphic representation of the data of the relative binding affinities of compound MSDC-0602, the metabolite of MSDC-0602, and the two insulin sensitizers rosiglitazone and pioglitazone with the MPC.

FIGS. 9A-9B. Shows the levels of change from baseline in ALT (FIG. 9A) and AST (FIG. 9B) by visit over 6 months of treatment with MSDC-0602K (62.5 mg, 125 mg, 250 mg).

FIG. 10. Show NASH pathology after 19 weeks of high fat, high cholesterol, and high sugar diet in mice, as compared to mice who were fed a normal diet.

FIG. 11. Shows the participant flow through the EMMINENCE study.

FIGS. 12A-12D. Shows the effects of MSDC-0602K on changes of glucose metabolism markers from baseline to 6 and 12 months: glucose (FIG. 12A), HOMA-IR (FIG. 12B), HbA1c (FIG. 12C), and fasting insulin (FIG. 12D). Mean changes relative to placebo with SEs adjusted for age, sex, diabetes mellitus (yes/no), Vitamin E>=400 IU (yes/no), baseline fibrosis score (F1 vs. F2/F3) and baseline value of marker.

FIGS. 13A-13D. Shows the effects of MSDC-0602K on liver enzymes: ALT (FIG. 13A), AST (FIG. 13B), alkaline phosphatase (FIG. 13C), and GGT (FIG. 13D). Mean changes relative to placebo with SEs adjusted for age, sex, diabetes mellitus (yes/no), Vitamin E>=400 IU (yes/no), baseline fibrosis score (F1 vs. F2/F3) and baseline value of marker.

FIGS. 14A-B. Shows the effects on apoptosis and fibrosis biomarkers of MSDC-0602K 125 and 250 mg doses combined compared to placebo. Mean Z-score differences from placebo and associated 95% CIs derived from changes to 6 months (A) and 12 months (B) are presented.

FIGS. 15A-15C. Shows the associations of changes in fasting insulin 15(A), HbA1c 15(B), and AST 15(C) with changes in weight from baseline to 12 months comparing combined 125 and 250 mg MSDC-0602K doses (blue) with placebo (gray). Linear regression lines with 95% confidence bounds are shown.

FIG. 16. Shows the effect in combined 125 and 250 mg MSDC-0602K dose groups versus placebo on ALT (FIG. 16A), AST (FIG. 16B), HbA1C (FIG. 16C), fasting insulin (FIG. 16D), and liver histology endpoints in patients without (FIG. 16E) versus with (FIG. 16F) AST>27 U/L and HgbA1C>6%. Mean changes relative to placebo with SEs adjusted for age, sex, diabetes mellitus (yes/no), Vitamin E>=400 IU (yes/no), baseline fibrosis score (F1 vs. F2/F3) and baseline value of marker are presented in FIGS. 16A-16D.

FIG. 17. Shows an improvement in nonalcoholic fatty liver disease activity score (NAS) after receiving MSDC-0602K in patients with PNPLA3 GG genotype. In 125 mg MSDC-0602K dose group with PNPLA3 GG genotype, about 28.6% of the patients improved nonalcoholic fatty liver disease activity score (NAS) score and in 250 mg MSDC-0602K dose group with PNPLA3 GG genotype, about 34.5% as compared to 17.6% in the placebo group.

FIG. 18. Shows the association of changes in aspartate aminotransferase (AST; FIGS. 18A-18B), hemoglobin A1c (HbA1c; FIGS. 18C-18D), and cytokeratin-18 (CK-18; FIGS. 18E-18F) with changes in histological parameters in 17 NASH clinical trials.

FIG. 19. Shows the association of the treatment ratio for changes in AST (FIGS. 19A-19B), HbA1c (FIGS. 19C-19D), and CK-18 (FIGS. 19E-19F) with the treatment effect on histological parameters in 17 NASH clinical trials.

FIG. 20. Shows the association of changes in AST, HbA1c, and CK-18 with change in NAS and rates of histological endpoints in 17 NASH clinical trials. Specifically, FIG. 20A shows these changes in AST and HbA1c. FIG. 20B shows these changes in CK-18.

FIG. 21. Shows the association of the treatment ratio for changes in AST, HbA1c, and CK-18 with the treatment effect for change in NAS and histological endpoints in 17 NASH clinical trials. Specifically, FIG. 21A shows these changes in AST and HbA1c. FIG. 21B shows these changes in CK-18.

FIGS. 22A-22E. Show the ROC curves for associations of baseline-adjusted 12-month changes with (left side) Fibrosis improvement without worsening of NASH and (right side) NASH resolution without worsening of fibrosis in the EMMINENCE trial: (FIG. 22A) ELF, (FIG. 22B) Fibrotest, (FIG. 22C) FIB-4, (FIG. 22D) Fibroscan stiffness, (FIG. 22E) weighted average Z of AST, HbA1c, and CK-18.

FIG. 23. Shows a flow chart of systematic review of literature.

FIG. 24. Shows an association of percent change from baseline in markers with change in ballooning.

FIG. 25. Shows an association of treatment ratio of change from baseline in markers with the treatment effect of change in ballooning.

FIG. 26. Shows an association of percent change from baseline in markers with rate of improvement by >=1 stage in ballooning.

FIG. 27. Shows an association of treatment ratio of change from baseline in markers with the treatment effect on improvement by >=1 stage in ballooning.

FIG. 28. Shows an association of percent change from baseline in markers with change in steatosis.

FIG. 29. Shows an association of treatment ratio of change from baseline in markers with the treatment effect of change in steatosis.

FIG. 30. Shows an association of percent change from baseline in markers with rate of improvement by >=1 stage in steatosis.

FIG. 31. Shows an association of treatment ratio of change from baseline in markers with the treatment effect on improvement by >=1 stage in steatosis.

FIG. 32. Shows an association of percent change from baseline in markers with change in inflammation.

FIG. 33. Shows an association of treatment ratio of change from baseline in markers with the treatment effect of change in inflammation.

FIG. 34. Shows an association of percent change from baseline in markers with rate of improvement by >=1 stage in inflammation.

FIG. 35. Shows an association of treatment ratio of change from baseline in markers with the treatment effect on improvement by >=1 stage in inflammation.

FIG. 36. Shows an association of percent change from baseline in markers with change in fibrosis.

FIG. 37. Shows an association of treatment ratio of change from baseline in markers with the treatment effect of change in fibrosis.

FIG. 38. Shows an association of percent change from baseline in markers with rate of improvement by >=1 stage in fibrosis.

FIG. 39. Shows an association of treatment ratio of change from baseline in markers with the treatment effect on improvement by >=1 stage in fibrosis.

FIG. 40. Shows an association of percent change from baseline in markers with change in NAS.

FIG. 41. Shows an association of treatment ratio of change from baseline in markers with the treatment effect of change in NAS.

FIG. 42. Shows an association of percent change from baseline in markers with rate of improvement by >=1 stage in NAS.

FIG. 43. Shows an association of treatment ratio of change from baseline in markers with the treatment effect on improvement by >=1 stage in NAS.

FIG. 44. Shows an association of percent change from baseline in markers with rate of resolution of NASH.

FIG. 45. Shows an association of treatment ratio of change from baseline in markers with the treatment effect on resolution of NASH.

FIG. 46. Shows an association of percent change from baseline in markers with rate of the endpoint improvement of fibrosis with no worsening of NASH.

FIG. 47. Shows an association of treatment ratio of change from baseline in markers with the treatment effect on endpoint improvement of fibrosis with no worsening of NASH.

### DETAILED DESCRIPTION

Provided herein are, for example, compounds and compositions that modulate the MPC and have reduced binding and activation of the nuclear transcription factor PPARγ. Also provided herein are, for example, methods of treating or preventing a metabolic inflammation-mediated disease or disorder (e.g., diabetes mellitus type II), metabolic syndrome, non-alcoholic fatty liver disease (NAFLD), and/or non-alcoholic steatohepatitis (NASH).

NASH is characterized by a high degree of liver damage and can lead to cirrhosis, hepatocellular carcinoma, liver failure and the need for liver transplant and, potentially, liver-related death. The prevalence of NASH is growing globally. In the United States alone, an estimated 16 million people have NASH, a number which is projected to increase to 27 million by the year 2030. It is estimated that approximately half of NASH patients also have Type II diabetes, and these patients are at a high risk for poor clinical outcomes. There are currently no approved therapies for the treatment of NASH.

NASH is a rapidly growing cause of end-stage liver disease and liver transplant. Currently, NASH is the second leading cause of liver transplants in the United States. The availability of liver donors is extremely limited and the economic burden is severe. In 2017, the costs per transplant were estimated to be approximately $812,500 in the United States. Additionally, in patients with several comorbidities, liver transplant may not be a viable option, or it may not equate to a beneficial outcome.

NASH is currently underdiagnosed, and is often discovered incidentally, for example by blood tests showing elevated liver enzyme levels. NASH patients may be asymptomatic or suffer from fatigue, with other symptoms occurring as the liver disease advances. Diagnosis of NASH is based on the exclusion of other reasons for liver disease, such as the use of medications, viral hepatitis or excessive use of alcohol, followed by non-invasive imaging tests, such as ultrasound, Computed Tomography scan, or CT scan, and Magnetic Resonance Imaging, or MRI. Liver biopsy is currently the standard procedure for the diagnosis of NASH.

There are currently no therapies approved for the treatment of NASH, creating a significant unmet need for safe and efficacious pharmacological options. Current options, which include lifestyle modification and bariatric surgery, have significant limitations.

Lifestyle modifications consisting of diet, exercise and weight loss are advocated to treat patients with NASH. While these modifications may address the underlying overnutrition in NASH patients, the weight loss goal of approximately 10% that is recommended for patients with NASH is difficult to achieve and sustain due to poor adherence. For example, in a large prospective study evaluating the impact of lifestyle intervention on NASH resolution and fibrosis, only 30% achieved meaningful weight loss, and even fewer sustained the reported weight loss over time.

Bariatric surgery is an effective treatment option for individuals who are severely obese. Although surgery provides for long-term weight loss, resolution of obesity-associated diseases in most patients as well as regression and/or histologic improvement of NASH, it is expensive and involves significant risks which make it impractical for widespread use.

NASH is the liver manifestation of metabolic syndrome, a constellation of disorders that includes insulin resistance, Type II diabetes and obesity. Overnutrition, wherein available nutrients exceed energy needs, is a major driver of metabolic syndrome leading to an unhealthy imbalance in metabolic signals. A diet rich in processed foods with high fat and sugar content, and a sedentary lifestyle, results in a state of overnutrition which drives pathological metabolic processes in the liver. These disruptive processes may lead to excess fat deposition, inflammation, insulin resistance and cellular damage. Over time, liver cells may be replaced by scar tissue, or fibrosis, and this process may ultimately lead to cirrhosis. At this point in the disease progression, the diminished liver function may necessitate a liver transplantation or lead to liver cancer, and potentially liver-related death.

Key disturbances in metabolism caused by overnutrition are mediated by the mitochondrial pyruvate carrier (MPC), which regulates the rate of mitochondrial pyruvate metabolism. In the setting of overnutrition, an excess of pyruvate, an energy source for cells, is rapidly transported into the mitochondria through the MPC, leading to the modification of multiple downstream pathways including transcription factors such as peroxisome proliferator-activated receptors, or PPARs. Downstream effects of this include insulin resistance, increased fat storage, decreased fat oxidation, inflammation, cell damage and fibrosis.

The MPC is a recently discovered protein complex in the inner mitochondrial membrane that mediates the rate of entry of pyruvate—an end product of carbohydrate metabolism and an important source of energy for the cell—into the mitochondria where subsequent oxidative metabolism occurs. This complex is present in the mitochondria of every cell in the body and orchestrates downstream signals that coordinate the cellular machinery, enzymatic pathways and gene expression with the nutritional state and energy need. In animal studies, liver-specific knockout of the MPC has been shown to protect against liver damage, particularly fibrosis, otherwise caused by overnutrition.

Modifications of the MPC affect downstream pathways that regulate multiple cell functions. For example, slowing the entry of pyruvate into the mitochondrion results in an increase in the metabolism of amino acids such as alanine, aspartate and glutamate. Experimental NASH therapies, that function as insulin sensitizers (e.g., pioglitazone) show detrimental side effects including edema, unnecessary weight gain, and bone loss, that are thought to be caused by antagonism of the nuclear transcription factor, PPARγ. Other experimental NASH therapies (e.g., rosiglitazone) that have been shown to have reduced binding affinity to PPARγ, are less efficacious in subjects with NASH, particularly for the treatment of fibrosis in NASH patients. The present compositions and methods provide a standalone NASH therapy that is a highly specific modulator of the MPC with characterized by a significant reduction in binding affinity to PPARγ.

Aspects disclosed herein provide compounds of structural Formula (I), or a pharmaceutically acceptable salt thereof, modulates the activity of the MPC can thus exert pleiotropic pharmacology in the context of overnutrition. In some embodiments, MSDC-0602K, modulates the activity of the MPC and can exert pleiotropic pharmacology in the context of overnutrition. Disclosed herein, in some embodiments, are methods of treating NASH, the treatment comprising administering a compound of structural Formula (I), or a pharmaceutically acceptable salt thereof. In some embodiments, the compound of structural Formula (I), or a pharmaceutically acceptable salt thereof, is MSDC-0602K. In some embodiments, MSDC-0602K is administered as a monotherapy.

MSDC-0602K is a second generation TZD that selectively binds to the MPC and modulates the entry of pyruvate into the mitochondria. Unlike, the first generation TZDs, which are approved for the treatment of Type II diabetes, MSDC-0602K minimizes direct agonism of PPARγ, thereby reducing adverse effects caused by direct agonism of PPARγ. Thus, MSDC-0602K demonstrates the beneficial effects observed with first generation TZDs, but without the adverse effects that limit the use of first generation TZDs. By selectively targeting the MPC—the initial point of metabolic dysfunction, rather than downstream pathways, MSDC-0602K addresses the core pathology of NASH, insulin resistance, and Type II diabetes.

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and includes mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl. An alkoxy is an alkyl attached to the remainder of the molecule via an oxygen linker (—O—).

As used herein, an “alkenyl” group refers to an aliphatic carbon group that contains 2-8 (e.g., 2-12, 2-6, or 2-4) carbon atoms and at least one double bond. Like an alkyl group, an alkenyl group can be straight or branched. Examples of an alkenyl group include, but are not limited to allyl, isoprenyl, 2-butenyl, and 2-hexenyl. An alkenyl group can be optionally substituted with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, heterocycloaliphaticamino, or aliphaticsulfonylamino], sulfonyl [e.g., alkyl-SO₂—, cycloaliphatic-SO₂—, or aryl-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-12, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl [e.g., aliphaticsulfanyl or cycloaliphaticsulfanyl], sulfinyl [e.g., aliphaticsulfinyl or cycloaliphaticsulfinyl], sulfonyl [e.g., aliphatic-SO₂—, aliphaticamino-SO₂—, or cycloaliphatic-SO₂—], amido [e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl], urea, thiourea, sulfamoyl, sulfamide, alkoxycarbonyl, alkylcarbonyloxy, cycloaliphatic, heterocycloaliphatic, aryl, heteroaryl, acyl [e.g., (cycloaliphatic)carbonyl or (heterocycloaliphatic)carbonyl], amino [e.g., aliphaticamino], sulfoxy, oxo, carboxy, carbamoyl, (cycloaliphatic)oxy, (heterocycloaliphatic)oxy, or (heteroaryl)alkoxy

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

As used herein, an “amido” encompasses both “aminocarbonyl” and “carbonylamino”. These terms when used alone or in connection with another group refer to an amido group such as —N(R^{X})—C(O)—R^{Y} or —C(O)—N(R^{X})2, when used terminally, and —C(O)—N(R^{X})— or —N(R^{X})—C(O)— when used internally, wherein RX and RY are defined below. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—. R^{X} has the same meaning as defined above.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic [e.g., alkyl, alkenyl, or alkynyl, including carboxyalkyl, hydroxyalkyl, or haloalkyl such as trifluoromethyl], cycloaliphatic [e.g., cycloalkyl or cycloalkenyl], (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido [e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino], cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

As used herein, a “carbamoyl” group refers to a group having the structure —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X}, when used as a terminal group; or —OC(O)— or —C(O)O— when used as an internal group.

As used herein, a “haloaliphatic” group refers to an aliphatic group substituted with 1-3 halogens. For instance, the term haloalkyl includes the group —CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)— when used internally, wherein R^{X} has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R^{X} when used terminally and —S(O)₂— when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂— or the like.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidine” group refers to the structure —N═C(N(R^{X}R^{Y}))N(R^{X}R^{Y}) or —NR^{X}—C(═NR^{X})NR^{X}R^{Y} wherein R^{X} and R^{Y} have been defined above.

As used herein, the term “amidino” group refers to the structure —C═(NR^{X})N(R^{X}R^{Y}) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[CQQ]ᵥ- where Q is independently a hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, and R₃, and other variables contained in formulae described herein encompass specific groups, such as alkyl and aryl. Unless otherwise noted, each of the specific groups for the variables R₁, R₂, and R₃, and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this disclosure are those combinations that result in the formation of stable or chemically feasible compounds.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring.

The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

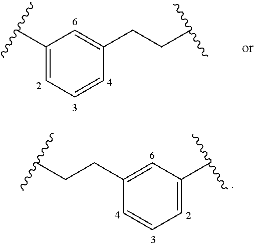 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃ —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR'SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.
(c) A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C6-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable moiety” as used herein refers to a moiety that can be covalently or noncovalently attached to a compound or biomolecule that can be detected for instance, using techniques known in the art. In embodiments, the detectable moiety is covalently attached. The detectable moiety may provide for imaging of the attached compound or biomolecule. The detectable moiety may indicate the contacting between two compounds. Exemplary detectable moieties are fluorophores, antibodies, reactive dies, radio-labeled moieties, magnetic contrast agents, and quantum dots. Exemplary fluorophores include fluorescein, rhodamine, GFP, coumarin, FITC, Alexa fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of a compound to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

An “MPC modulator” refers to a compound (e.g., compounds described herein) that directly or indirectly modulate the activity of the MPC when compared to a control, such as absence of the compound or a compound with known inactivity.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. In embodiments, the terms “polypeptide,” “peptide,” and “protein,” used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences, with or without N-terminus methionine residues; immunologically tagged proteins; and the like.

A polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway (e.g., MAP kinase pathway).

As defined herein, the term “activation”, “activate”, “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc., refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist. In embodiments, an agonist is a molecule that interacts with a target to cause or promote an increase in the activation of the target. In embodiments, activators are molecules that increase, activate, facilitate, enhance activation, sensitize, or up-regulate, e.g., a gene, protein, ligand, receptor, or cell.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease.

As used herein “metabolic inflammation-mediated disease or disorder” refers to disease states where metabolic inflammation is the basis of the pathology. Metabolic inflammation-mediated disease or disorder are diseases or disorders resulting from metabolic inflammation, including but not limited to hypertension, diabetes (e.g., diabetes mellitus type II), diabetes, metabolic syndrome, all aspects of insulin resistance associated with metabolic syndrome (including dyslipidemia and central obesity as well as fatty liver disease and NASH). In some embodiments, the metabolic inflammation-mediated disease or disorder comprises an “inflammatory disease” or and “autoimmune disease,” as described herein.

As used herein “metabolic syndrome” is a clustering of at least three of the five following medical conditions: abdominal obesity, high blood pressure, high blood sugar, high serum triglycerides and low high-density lipoprotein (HDL) levels and insulin resistance.

As used herein “non-alcoholic fatty liver disease” and “NAFLD” are interchangeable and refer to fatty liver, which occurs when fat is deposited (steatosis) in the liver due to causes other than excessive alcohol use. Non-alcoholic fatty liver disease (NAFLD) may be related to insulin resistance and the metabolic syndrome.

As used herein “non-alcoholic steatohepatitis” and “NASH” are interchangeable and refers to the a form of non-alcoholic fatty liver disease (NAFLD) as defined by histopathology, particularly hepatocyte ballooning and fibrotic scarring. Exemplary diseases and phenotypes associated with NASH include but are not limited to fibrosis, cirrhosis, hepatocellular carcinoma (HCC), liver failure, the need for a liver transplant, portal hypertension, esophageal varicies in between cirrhosis and HC, heart failure, myocardial infarcts, coronary and peripheral vascular disease and stroke.

As used herein, the term “inflammatory disease” refers to a disease or condition characterized by aberrant inflammation (e.g. an increased level of inflammation compared to a control such as a healthy person not suffering from a disease). Exam pies of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis. Such conditions are frequently inextricably intertwined with other diseases, disorders and conditions. A non-limiting list of inflammatory-related diseases, disorders and conditions which may, for example, be caused by inflammatory cytokines, include, arthritis, kidney failure, lupus, asthma, psoriasis, colitis, pancreatitis, allergies, fibrosis, surgical complications (e.g., where inflammatory cytokines prevent healing), anemia, and fibromyalgia. Other diseases and disorders which may be associated with chronic inflammation include Alzheimer's disease, congestive heart failure, stroke, aortic valve stenosis, arteriosclerosis, osteoporosis, Parkinson's disease, infections, inflammatory bowel disease (IBD), allergic contact dermatitis and other eczemas, systemic sclerosis, transplantation and multiple sclerosis. Some of the aforementioned diseases, disorders and conditions for which a compound (e.g., a MPC modulator) of the present disclosure may be particularly efficacious (due to, for example, limitations of current therapies) are described in more detail hereafter.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

The term “prediabetes,” refers to a condition characterized by a susceptibility to develop diabetes in a subject. In some instances, a subject is “prediabetic” provided an increase in biomarkers indicating a presence or severity of hyperglycemia are detected in a sample obtained from the subject, as compared to a reference level. In some instances, the biomarker comprises bemoglobin A1c (HbA1c).

The terms “treating,” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. As indicated above, the prevention may be complete (no detectable symptoms) or partial, such that fewer symptoms are observed than occurs absent treatment. In embodiments, prevent refers to slowing the progression of the disease, disorder or condition or inhibiting progression thereof to a harmful or otherwise undesired state.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In some embodiments, the subject carries both copies of risk allele of PNPLA3 rs738409-G. In some embodiments, the subject carries both copies of PNPLA3 rs738409-C allele. In some embodiments, the subject carries one copy of the risk allele of PNPLA3 rs738409-G and one copy of PNPLA3 rs738409-C.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function enhancing amount,” as used herein, refers to the amount of agonist required to enhance the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). The therapeutically effective amount can be ascertained by measuring relevant physiological effects, and it can be adjusted in connection with the dosing regimen and diagnostic analysis of the subject's condition, and the like. By way of example, measurement of the serum level of a MPC modulator (or, e.g., a metabolite thereof) at a particular time post-administration may be indicative of whether a therapeutically effective amount has been administered.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Adjusting the dose to achieve maximal therapeutic window efficacy or toxicity in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent, chemotherapeutic, or treatment for a neurodegenerative disease). The compound of the disclosure can be administered alone or can be coadministered to the patient.

Coadministration is meant to include simultaneous or sequential administration of the compound individually or in combination (more than one compound or agent). Thus, the preparations can also be combined, when desired, with other active substances (e.g. to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. These patents are incorporated herein by reference for such disclosure. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

By “co-administer” it is meant that a composition described herein is administered at the same time, just prior to, or just after the administration of one or more additional therapies. The compounds of the disclosure can be administered alone or can be coadministered to the patient. Coadministration is meant to include simultaneous or sequential administration of the compounds individually or in combination (more than one compound). The compositions of the present disclosure can be delivered transdermally, by a topical route, or formulated as applicator sticks, solutions, suspensions, emulsions, gels, creams, ointments, pastes, jellies, paints, powders, and aerosols.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds (e.g., MPC modulators) disclosed herein can be administered by any acceptable route, such oral, intraadiposal, intraarterial, intraarticular, intracranial, intradermal, intralesional, intramuscular, intranasal, intraocularal, intrapericardial, intraperitoneal, intrapleural, intraprostatical, intrarectal, intrathecal, intratracheal, intratumoral, intraumbilical, intravaginal, intravenous, intravesicullar, intravitreal, liposomal, local, mucosal, parenteral, rectal, subconjunctival, subcutaneous, sublingual, topical, transbuccal, transdermal, vaginal, in cremes, in lipid compositions, via a catheter, via a lavage, via continuous infusion, via infusion, via inhalation, via injection, via local delivery, via localized perfusion, bathing target cells directly, or any combination thereof.

The compounds (e.g., MPC modulators) disclosed herein may be administered once daily until study reached endpoint. The immune modulator disclosed herein may be administered at least three times but in some studies four or more times depending on the length of the study and/or the design of the study.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” refers to a composition that increases or decreases the level of a target molecule or the function of a target molecule or the physical state of the target of the molecule. In some embodiments, a MPC-associated disease modulator is a compound that reduces the severity of one or more symptoms of a disease associated with MPC and/or PPAR (e.g., metabolic inflammation-mediated disease or disorder (e.g., diabetes mellitus type II), metabolic syndrome, non-alcoholic fatty liver disease (NAFLD), and/or non-alcoholic steatohepatitis (NASH)). A MPC modulator is a compound that increases or decreases the activity or function or level of activity or level of function of MPC. A modulator may act alone, or it may use a cofactor, e.g., a protein, metal ion, or small molecule. Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an activator

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. In embodiments, the terms “modulate,” “modulation” and the like refer to the ability of a molecule (e.g., an activator or an inhibitor) to increase or decrease the function or activity of MPC or PPAR, either directly or indirectly, relative to the absence of the molecule.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The phrase “in a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like.

“Substantially pure” indicates that a component makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition (percentage in a weight per weight basis).

The terms “specifically binds” and “selectively binds”, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two-fold greater, at least 10-times greater, at least 20-times greater, or at least 100-times greater than the affinity with any other antibody, or binding composition derived therefrom. In embodiments, the antibody will have an affinity that is greater than about 10⁹ liters/mol, as determined by, e.g., Scatchard analysis (Munsen, et al. (1980) Analyt. Biochem. 107:220-239).

The terms “DNA,” “nucleic acid,” “nucleic acid molecule,” “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

As used herein, the terms “variants” and “homologs” are used interchangeably to refer to amino acid or nucleic acid sequences that are similar to reference amino acid or nucleic acid sequences, respectively. The term encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Thus, variants and homologs encompass naturally occurring amino acid and nucleic acid sequences encoded thereby and their isoforms, as well as splice variants of a protein or gene. The terms also encompass nucleic acid sequences that vary in one or more bases from a naturally-occurring nucleic acid sequence but still translate into an amino acid sequence that corresponds to the naturally-occurring protein due to degeneracy of the genetic code. Non-naturally-occurring variants and homologs include polypeptides and nucleic acids that comprise a change in amino acid or nucleotide sequence, respectively, where the change in sequence is artificially introduced (e.g., muteins); for example, the change is generated in the laboratory by human intervention (“hand of man”). Therefore, non-naturally occurring variants and homologs may also refer to those that differ from the naturally-occurring sequences by one or more conservative substitutions and/or tags and/or conjugates.

The term “muteins” as used herein refers broadly to mutated recombinant proteins. These proteins usually carry single or multiple amino acid substitutions and are frequently derived from cloned genes that have been subjected to site-directed or random mutagenesis, or from completely synthetic genes.

### II. Compounds

In an aspect provided herein, is a compound having structural Formula (I):

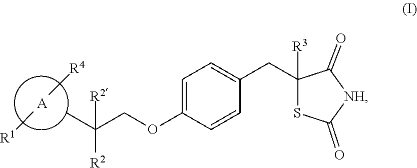

or a pharmaceutically acceptable salt thereof wherein R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, R³ is hydrogen.

In other embodiments, R⁴ is independently hydrogen, methyl, or —OR^{4A}; and R^{4A} is independently methyl, ethyl, isopropyl, —CHF₂, or —CF₃. In some embodiments, R⁴ is hydrogen.

In further embodiments, R¹ is hydrogen, halogen or —OR^{1A}; and R^{1A} is substituted or unsubstituted alkyl. In another embodiments R^{1A} is substituted or unsubstituted C₁-C₃alkyl. In other embodiments, R^{1A} is —CHF₂ or —CF₃. In some embodiments, R¹ is hydrogen. In a further embodiment, R¹ is —OR^{1A}; and R^{1A} is substituted or unsubstituted alkyl. In other embodiments, R¹ is halogen. In some embodiments, R¹ is —F or —Cl. In further embodiments, R¹ is attached to the para or meta position of the phenyl. In other embodiments, R¹ is attached to the ortho or meta position of the phenyl. In further embodiments, R¹ is attached to the meta position of the phenyl.

In some embodiments, R^{2′} is hydrogen. In some other embodiments, R² is hydroxyl. In further embodiments, R² and R^{2′} are joined to form oxo.

In some embodiments, the compound of Formula (I) is:

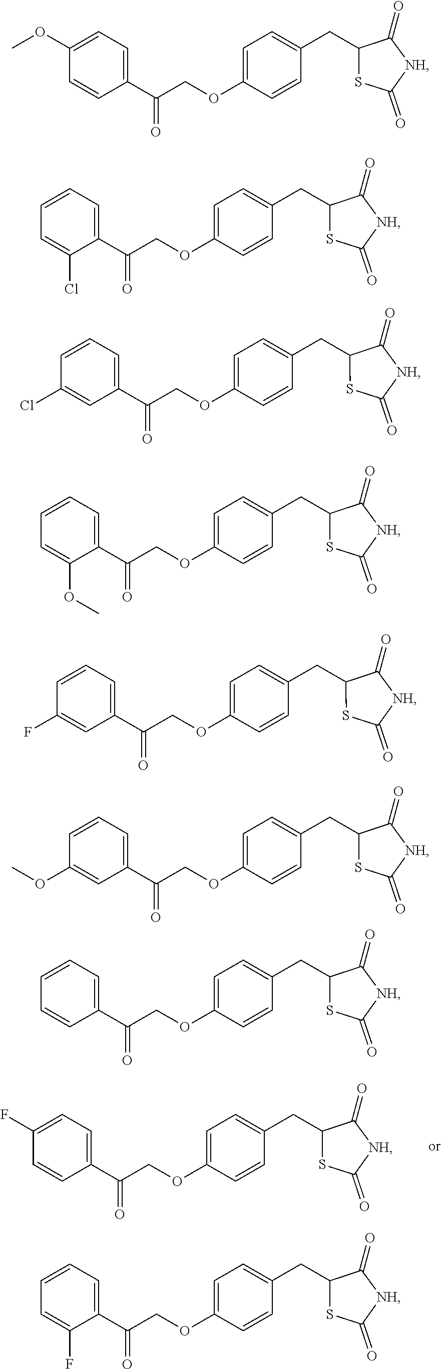

or a pharmaceutically acceptable salt thereof.

In another embodiment, the compound of Formula (I) is:

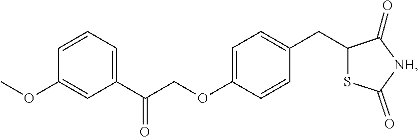 [SMILES: COc1cccc(C(=O)COc2ccc(CC3SC(=O)NC3=O)cc2)c1]

or a pharmaceutically acceptable salt thereof, also termed MSDC-0602. In some embodiments, the potassium salt of

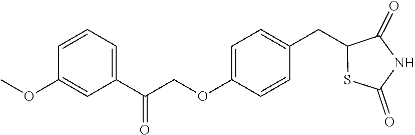 [SMILES: COc1cccc(C(=O)COc2ccc(CC3SC(=O)NC3=O)cc2)c1]

### is MSDC-0602K.

### III. Compositions

In an aspect is provided a composition, including (i) a compound having structural Formula (I):

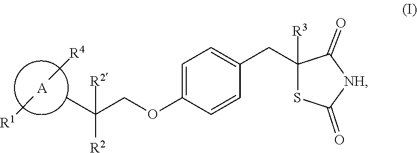

or a pharmaceutically acceptable salt thereof. In the compound having structural Formula (I), R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a pharmaceutical composition, including a compound as described herein, including embodiments, or the structural Formula (I), and at least one pharmaceutically acceptable excipient.

In embodiments, the pharmaceutically acceptable salt is a potassium salt.

The compounds (e.g., MPC or PPAR modulator(s)) of the present disclosure may be in the form of compositions suitable for administration to a subject. In general, such compositions are “pharmaceutical compositions” comprising a compound (e.g., MPC or PPAR modulator(s)) and one or more pharmaceutically acceptable or physiologically acceptable diluents, carriers or excipients. In certain embodiments, the compounds (e.g., MPC or PPAR modulator(s)) are present in a therapeutically acceptable amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein.

The pharmaceutical compositions containing the active ingredient (e.g., a modulator of MPC or PPAR function) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture thereof. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a MPC or PPAR modulator contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a MPC or PPAR modulator, including implants (e.g., implantable pumps) and catheter systems, slow injection pumps and devices, all of which are well known to the skilled artisan.

Depot injections, which are generally administered subcutaneously or intramuscularly, may also be utilized to release the compound (e.g., MPC or PPAR modulator) disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, N.J.) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin).

The present disclosure contemplates the administration of the compound (e.g., MPC or PPAR modulator) in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The compound (e.g., MPC or PPAR modulator) contemplated by the present disclosure may be in the form of any other suitable pharmaceutical composition (e.g., sprays for nasal or inhalation use) currently known or developed in the future.

### IV. Methods for Treating or Preventing Metabolic Inflammation-Mediated Disease or Disorder

Provided herein is a method of treating or preventing at least one metabolic inflammation-mediated disease or disorder, comprising administering to a subject in need thereof:

a therapeutically effective amount of a compound of structural Formula (I):

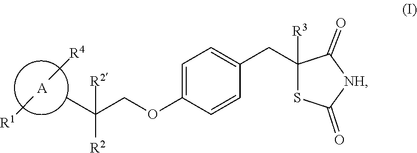

or a pharmaceutically acceptable salt thereof wherein R¹ is independently hydrogen, halogen, substituted or unsubstituted alkyl, or —OR^{1A}; R² is halogen, hydroxyl, or optionally substituted aliphatic; R^{2′} is hydrogen, or R² and R^{2′} may optionally be joined to form oxo; R³ is hydrogen or deuterium; R⁴ is independently hydrogen, halogen, substituted or unsubstituted alkyl, or —OR^{4A}; A is phenyl; R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, R³ is hydrogen.

In other embodiments, R⁴ is independently hydrogen, methyl, or —OR^{4A}; and R^{4A} is independently methyl, ethyl, isopropyl, —CHF₂, or —CF₃. In further embodiments, R⁴ is hydrogen.

In some embodiments, R¹ is hydrogen, halogen or —OR^{1A}; and R^{VA} is substituted or unsubstituted alkyl. In other embodiments, R^{VA} is substituted or unsubstituted C₁-C₃alkyl. In some embodiments, R^{1A} is —CHF₂ or —CF₃. In further embodiments, R¹ is hydrogen. In yet further embodiments, R¹ is —OR^{1A}; and R^{1A} is substituted or unsubstituted alkyl. In some embodiments, R¹ is halogen. In some other embodiments, R¹ is —F or —Cl. In further embodiments, R¹ is attached to the para or meta position of the phenyl; or R¹ is attached to the ortho or meta position of the phenyl. In yet further embodiments, R¹ is attached to the meta position of the phenyl.

In some other embodiments, R^{2′} is hydrogen. In further embodiments, R² is hydroxyl. In some embodiments, R² and R^{2′} are joined to form oxo.

In other embodiments, the compound of Formula (I) is:

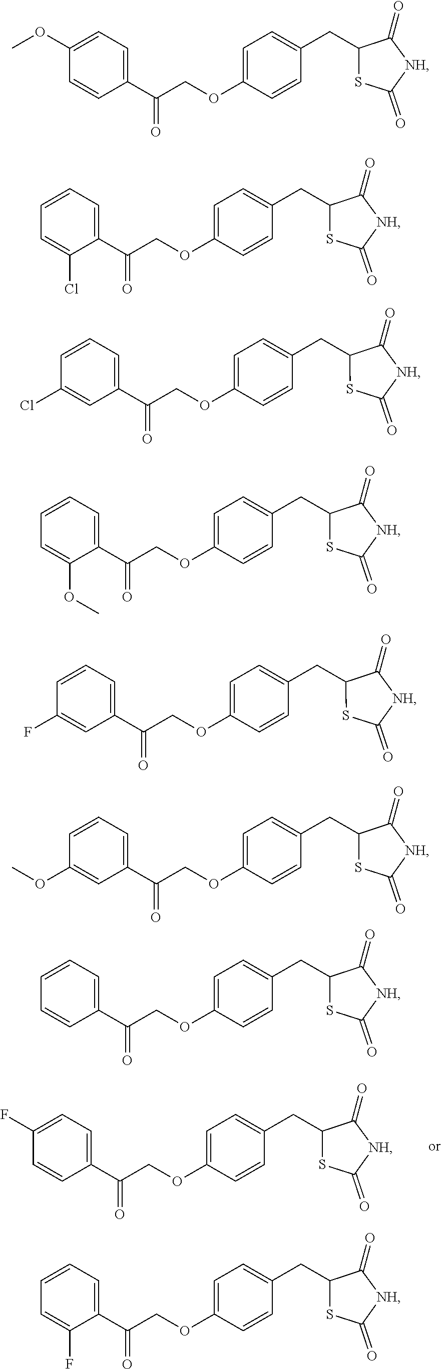

or a pharmaceutically acceptable salt thereof.

In further embodiments, the compound of Formula (I) is:

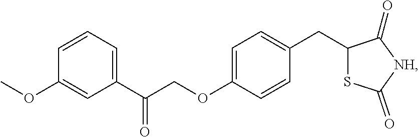 [SMILES: COc1cccc(C(=O)COc2ccc(CC3SC(=O)NC3=O)cc2)c1]

or a pharmaceutically acceptable salt thereof.

In some embodiments, the pharmaceutically acceptable salt is a potassium salt.

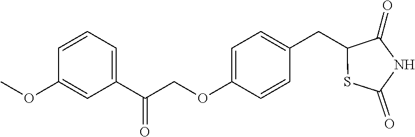 [SMILES: COc1cccc(C(=O)COc2ccc(CC3SC(=O)NC3=O)cc2)c1]

In some embodiments, the potassium salt of is MSDC-0602K.

In another embodiment, MSDC-0602K has the structure:

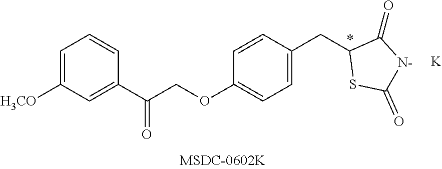

In embodiments, the compound of Formula (I), or a pharmaceutically acceptable salt thereof, is administered orally.

In embodiments, the compound of Formula (I), or a pharmaceutically acceptable salt thereof, is formulated as a tablet or capsule.

In other embodiments is a tablet comprising a compound of Formula (I), or a pharmaceutically acceptable potassium salt thereof; and excipients selected from anhydrous lactose, magnesium stearate, microcrystalline cellulose, sodium croscarmellose, povidone K-30, colloidal silicon dioxide and opadry. In further embodiments, the tablet comprises MSDC-0602K. In yet a further embodiment the tablet comprises about 62.5 mg of MSDC-0602K. In another embodiment, the tablet comprises about 125 mg of MSDC-0602K. In yet another embodiment, the tablet comprises about 250 mg of MSDC-0602K.

In one embodiment is a method of treating a patient having NASH and diabetes comprising administering to the patient about 125 mg of MSDC-0602K wherein the liver enzymes of the patient prior to treatment are elevated compared to a patient having a normal range of liver enzymes. In another embodiment the liver enzyme is ALT or AST. In another embodiment, is a method for treating a patient having NASH and diabetes comprising administering to the patient about 250 mg of MSDC-0602K wherein the administration results in significant reduction in HOMA-IR and/or HbA1c. In a further embodiment is a method for treating a patient having NASH and diabetes comprising administering to the patient about 125 mg of MSDC-0602K wherein the administration results in significant reduction in HOMA-IR and/or HbA1c. In another embodiment is a method for treating a patient having NASH and diabetes comprising administering to the patient about 62.5 mg of MSDC-0602K. In another embodiment is a method for treating a patient having NASH and diabetes comprising administering to the patient about 125 mg of MSDC-0602K. In yet another embodiment is a method for treating a patient having NASH and diabetes comprising administering to the patient about 250 mg of MSDC-0602K.

In yet a further embodiment is a method of treating a patient having NASH and diabetes comprising administering to the patient about 125 mg or 250 mg of MSDC-0602K once a day wherein the patient shows at least one of the following: improved liver status, improved glycemic control, improved outcomes such as cardiovascular, mortality, liver outcomes, and long-term outcomes.) In yet another embodiment, the amount administered to the patient is a single-dose administration. In yet a further embodiment, the patient is confirmed as having NASH by biopsy. In yet another embodiment, the diabetes is Type II diabetes. In another embodiment, the patient has met the ADA criteria for diabetes. In a further embodiment, the patient has been diagnosed by a physician as having diabetes. In another embodiment, the patient has met the classification and diagnosis of diabetes as described in Diabetes Care 2018; 41(Suppl. 1):S13-S27.

In yet another embodiment, the patient has features of fatty liver disease that leads one to believe they may have NASH.

In one embodiment the single dose formulation of 125 mg of MSDC-0602K provides the patient with an improved ALT enzyme levels as compared to 250 mg of MSDC-0602K. In a further embodiment, the single dose formulation of 250 mg of MSDC-0602K provides a faster effect on liver enzyme levels such as ALT and/or AST enzyme levels as compared to the 125 mg dosage formulation. In yet another embodiment the single dose formulation of 62.5 mg of MSDC-0602K provides good efficacy on patients requiring improved insulin sensitivity. In another embodiment is a method for treating a patient having NASH and diabetes comprising administering to the patient about 125 mg or about 250 mg of MSDC-0602K wherein the treatment produces improved cardiovascular outcome relative to a dosage amount of about 62.5 mg of MSDC-0602K despite a significant weight gain.

In one embodiment is a method for improving glycemic control in patients with Type II diabetes comprising administering to the patient about 125 mg of MSDC-0602K.

In one embodiment, the patient is administered MSDC-0602K in the morning. In another embodiment, the patient is administered MSDC-0602K in the evening. In a further embodiment, the patient is administered MSDC-0602K at night.

In one embodiment is a method for treating a patient having a higher than normal baseline of ALT and/or AST liver enzyme levels comprising administering to the patient a therapeutically effective amount of MSDC-0602K. In another embodiment, the therapeutically effective amount of MSDC-0602K is about 125 mg. In a further embodiment, the treatment results in a greater lowering of liver enzyme levels such as ALT and/or AST.

In one embodiment is a method of treating a patient having NASH and diabetes comprising administering to the patient about 62.5 mg of MSDC-0602K wherein the liver enzymes of the patient prior to treatment are elevated compared to a patient having a normal range of liver enzymes. In another embodiment the liver enzyme is ALT or AST. In another embodiment, is a method for treating a patient having NASH and diabetes comprising administering to the patient about 62.5 mg of MSDC-0602K wherein the administration results in significant reduction in HOMA-IR and/or HbA1c.

In one embodiment is a method of treating patients having NASH and diabetes comprising: measuring the circulating liver enzyme levels of a patient prior to initial treatment; treating the patient with a compound of Formula (I); measuring the circulating liver enzyme levels following the initial treatment; and determining whether treatment with a compound of Formula (I) should continue based on the liver enzyme levels as compared to a standard.

In another embodiment is a method described above wherein the compound of Formula (I) is MSDC-0602K. In a further embodiment is a method described above wherein the liver enzyme is ALT. In a further embodiment, the liver enzyme is AST.

In one embodiment is a method of treating a prediabetic patient comprising administering to the patient a therapeutically effective amount of MSDC-0602K. In another embodiment, the therapeutically effective amount is about 62.5 mg, about 125 mg, or about 250 mg.

In certain embodiments, in the treatment, prevention, or amelioration of one or more symptoms of the disorders, diseases, or conditions described herein (e.g., treatment or prevention of at least one metabolic inflammation-mediated disease or disorder), an appropriate dosage level of a compound of Formula (I), or a pharmaceutically acceptable salt, solvate, hydrate, or prodrug thereof generally is ranging from about 1 to 3000 mg, from about 1 to 2000 mg, from about 1 to 1000 mg, from about 1 to about 500 mg, from about 5 to about 500 mg, from about 5 to about 400 mg, from about 5 to about 300 mg, from about 5 to about 250 mg, from about 5 to about 125 mg or from about 62.5 to about 250 mg, which can be administered in single or multiple doses. In certain embodiments, in the treatment, prevention, or amelioration of one or more symptoms of the disorders, diseases, or conditions described herein (e.g., treatment or prevention of at least one metabolic inflammation-mediated disease or disorder), an appropriate dosage level of a compound of Formula (I), or a pharmaceutically acceptable salt, solvate, hydrate, or prodrug thereof is 62.5 mg, 125 mg, or 250 mg. In certain embodiments, the compound of Formula (I), or a pharmaceutically acceptable salt, solvate, hydrate, or prodrug thereof is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 300, 350, 400, or 500 mg. In certain embodiments, the compound of Formula (I), or a pharmaceutically acceptable salt, solvate, hydrate, or prodrug thereof is administered in an amount of about 60, 60.5, 61, 61.5, 62, 62.5, 63, 63.5, 64, 64.5, 65, 65.5, 66, 66.5, 67, 67.5, 68, 68.5, 69, 69.5, or 70 mg. In certain embodiments, the compound of Formula (I), or a pharmaceutically acceptable salt, solvate, hydrate, or prodrug thereof is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 300, 350, 400, or 500 mg/day. In certain embodiments, the compound of Formula (I), or a pharmaceutically acceptable salt, solvate, hydrate, or prodrug thereof is administered in an amount of about 60, 60.5, 61, 61.5, 62, 62.5, 63, 63.5, 64, 64.5, 65, 65.5, 66, 66.5, 67, 67.5, 68, 68.5, 69, 69.5, or 70 mg/day.

For oral administration, the pharmaceutical compositions provided herein can be formulated in the form of tablets or capsules containing from about 1.0 to about 1,000 mg of a compound of Formula (I), or a pharmaceutically acceptable salt, solvate, hydrate, or prodrug thereof. In certain embodiments, for oral administration, the pharmaceutical compositions provided herein can be formulated in the form of tablets or capsules containing about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 300, about 350, about 400, or about 500 mg of a compound of Formula (I), or a pharmaceutically acceptable salt, solvate, hydrate, or prodrug thereof for the symptomatic adjustment of the dosage to the patient to be treated. In certain embodiments, for oral administration, the pharmaceutical compositions provided herein can be formulated in the form of tablets or capsules containing about 60, 60.5, 61, 61.5, 62, 62.5, 63, 63.5, 64, 64.5, 65, 65.5, 66, 66.5, 67, 67.5, 68, 68.5, 69, 69.5, or 70 mg of a compound of Formula (I), or a pharmaceutically acceptable salt, solvate, hydrate, or prodrug thereof for the symptomatic adjustment of the dosage to the patient to be treated.

The pharmaceutical compositions can be administered on a regimen of one (1) to four (4) times per day, including once, twice, three times, and four times per day. In some embodiments, the compound of Formula (I), or an isotopic variant thereof or a pharmaceutically acceptable salt, solvate, hydrate, or prodrug thereof is administered once per day.

In some embodiments, the compound of Formula (I), or a pharmaceutically acceptable salt thereof, is administered in a dose of from about 60 mg to about 250 mg. In some embodiments, the compound of Formula (I), or a pharmaceutically acceptable salt thereof, is administered in a dose of about 62.5 mg. In other embodiments, the compound of Formula (I), or a pharmaceutically acceptable salt thereof, is administered in a dose of about 125 mg. In further embodiments, the compound of Formula (I), or a pharmaceutically acceptable salt thereof, is administered in a dose of about 250 mg. In certain embodiments, the compound of Formula (I), or a pharmaceutically acceptable salt thereof, is administered in a dose of about 125 mg or about 250 mg for a period of time comprising one month, two months, three months, four months, five months, six months, seven months, eight months, nine months, ten months, eleven months, or twelve months. Thereafter, in certain embodiments, the compound of Formula (I), or a pharmaceutically acceptable salt thereof, is administered in a dose of about 62.5 mg for a period of time comprising one year, two years, three years, four years, five years, six years, seven years, eight years, nine years, ten years, 20 years, 30 years, 40 years, 50 years, 60 years, 70 years, 80 years, or more. In a further embodiment, the compound of Formula (I), or a pharmaceutically acceptable salt thereof is administered in a dose of about 250 mg for a period of time followed by administration in a dose of about 125 mg thereafter. In yet a further embodiment, the compound of Formula (I), or a pharmaceutically acceptable salt thereof is administered in a dose of about 250 mg for a period of time followed by administration in a dose of about 62.5 mg thereafter. In another embodiment, the compound of Formula (I), or a pharmaceutically acceptable salt thereof is administered in a dose of about 125 mg for a period of time followed by administration in a dose of about 62.5 mg thereafter.

In other embodiments, the compound of Formula (I), or a pharmaceutically acceptable salt thereof, is administered daily. In embodiments, the compound of Formula (I), or a pharmaceutically acceptable salt thereof, is administered once daily.

Provided herein is a method of treating or preventing non-alcoholic fatty liver disease (NAFLD) and/or metabolic syndrome, comprising administering to a subject in need thereof a therapeutically effective amount of a compound as described herein, including embodiments, or the structural Formula (I), or a pharmaceutically acceptable salt thereof.

Provided herein is a method of treating or preventing non-alcoholic fatty liver disease (NAFLD) and/or at least one metabolic inflammation-mediated disease or disorder, comprising administering to a subject in need thereof a therapeutically effective amount of a pharmaceutical composition comprising:

a compound of structural Formula (I), or a pharmaceutically acceptable salt thereof:

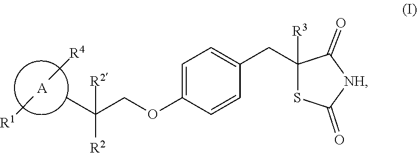

wherein R¹, R², R^{2′}, R³, and R⁴ are as described herein, including embodiments;

and

(iii) and a pharmaceutically acceptable.

In an aspect is provided a method of treating at least one metabolic inflammation-mediated disease or disorder, comprising administering to a subject in need thereof a therapeutically effective amount of a compound of structural Formula (I), or a pharmaceutically acceptable salt thereof:

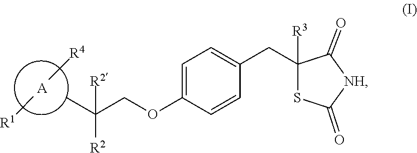

wherein R¹, R², R^{2′}, and R⁴ are as described herein, and R³ is deuterium.

In some embodiments, the subject has NAFLD. In other embodiments, the subject has at least one metabolic inflammation-mediated disease or disorder. In further embodiments, the subject has NAFLD and at least one metabolic inflammation-mediated disease or disorder. In some embodiments, the subject has (NAFLD) and/or metabolic syndrome and at least one metabolic inflammation-mediated disease or disorder. In other embodiments, the subject has diabetes mellitus, NAFLD, or metabolic syndrome, or any combination thereof. In other embodiments, the subject is suffering from obesity, non-alcoholic fatty liver disease (NAFLD), a metabolic inflammation-mediated disease or disorder, metabolic syndrome, or any combination thereof.

In some embodiments, the NAFLD is non-alcoholic steatohepatitis (NASH). In other embodiments, the subject has NASH with fibrosis.

In further embodiments, the at least one metabolic inflammation-mediated disease or disorder is diabetes mellitus type II.

In another aspect is provided a method of inhibiting hepatocyte mitochondrial pyruvate carrier (MPC), comprising contacting the MPC with a compound as described herein, including embodiments, or the structural Formula (I)

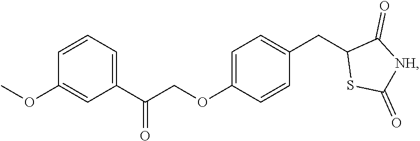 [SMILES: COc1cccc(C(=O)COc2ccc(CC3SC(=O)NC3=O)cc2)c1]

or a pharmaceutically acceptable salt thereof.

In one aspect is provided a method of inhibiting hepatocyte mitochondrial pyruvate carrier (MPC), comprising contacting the MPC with a compound as described herein, including embodiments, or the structural Formula (I)

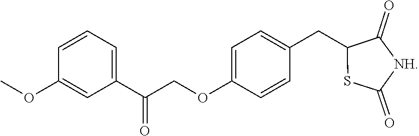 [SMILES: COc1cccc(C(=O)COc2ccc(CC3SC(=O)NC3=O)cc2)c1]

In embodiments, the hepatocyte is in vivo. In embodiments, the hepatocyte is a human hepatocyte.

In an aspect is provided a method of improving or increasing glucose tolerance and/or insulin sensitivity, comprising administering to a subject in need thereof a therapeutically effective amount of a compound as described herein, including embodiments, or the structural Formula (I)

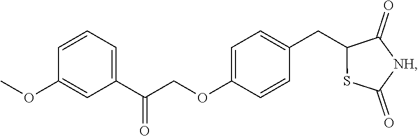 [SMILES: COc1cccc(C(=O)COc2ccc(CC3SC(=O)NC3=O)cc2)c1]

or a pharmaceutically acceptable salt thereof. In an aspect is provided a method of improving or increasing glucose tolerance and/or insulin sensitivity, comprising: administering to a subject in need thereof a therapeutically effective amount of a compound as described herein, including embodiments, or the structural Formula (I)

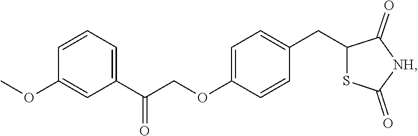 [SMILES: COc1cccc(C(=O)COc2ccc(CC3SC(=O)NC3=O)cc2)c1]

or a pharmaceutically acceptable salt thereof.

In an aspect is provided a method of treating or preventing a hepatic disease, disorder, or injury, comprising administering to a subject in need thereof a therapeutically effective amount of a compound as described herein, including embodiments, or the structural Formula (I)

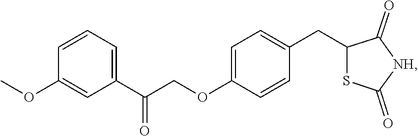 [SMILES: COc1cccc(C(=O)COc2ccc(CC3SC(=O)NC3=O)cc2)c1]

or a pharmaceutically acceptable salt thereof. In an aspect is provided a method of treating or preventing a hepatic disease, disorder, or injury, comprising: administering to a subject in need thereof a therapeutically effective amount of a compound as described herein, including embodiments, or the structural Formula (I)

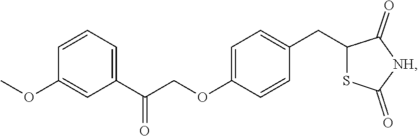 [SMILES: COc1cccc(C(=O)COc2ccc(CC3SC(=O)NC3=O)cc2)c1]

or a pharmaceutically acceptable salt thereof.

In embodiments, the hepatic disease, disorder, or injury is fibrosis.

In an aspect is provided a method of treating or preventing hepatocyte fibrogenesis, comprising administering to a subject in need thereof a therapeutically effective amount of a compound as described herein, including embodiments, or the structural Formula (I)

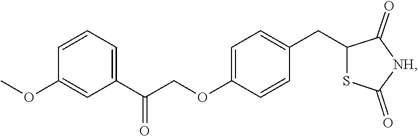 [SMILES: COc1cccc(C(=O)COc2ccc(CC3SC(=O)NC3=O)cc2)c1]

or a pharmaceutically acceptable salt thereof. In an aspect is provided a method of treating or preventing hepatocyte fibrogenesis, comprising: administering to a subject in need thereof a therapeutically effective amount of a compound as described herein, including embodiments, or the structural Formula (I)

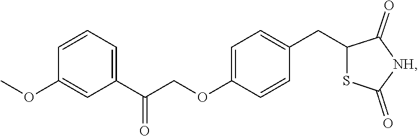 [SMILES: COc1cccc(C(=O)COc2ccc(CC3SC(=O)NC3=O)cc2)c1]

or a pharmaceutically acceptable salt thereof.

In an aspect provided herein, the present disclosure provides methods of reducing a level of alanine transaminase (ALT) and/or aspartate aminotransferase (AST) in a subject diagnosed with a nonalcoholic fatty liver disease, the method comprising administering to the subject a therapeutically effective amount of a compound of structural Formula (I)

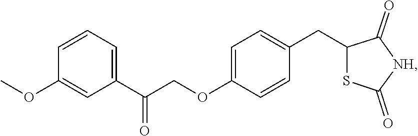 [SMILES: COc1cccc(C(=O)COc2ccc(CC3SC(=O)NC3=O)cc2)c1]

or a pharmaceutically acceptable salt thereof. Also provided, in some aspects, are methods of reducing a level of hemoglobin A1c (HbA1c) in a subject diagnosed with diabetes, the method comprising administering to the subject a therapeutically effective amount of a compound of structural Formula (I)

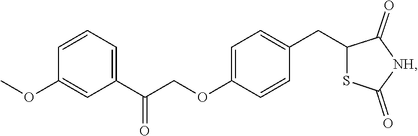 [SMILES: COc1cccc(C(=O)COc2ccc(CC3SC(=O)NC3=O)cc2)c1]

or a pharmaceutically acceptable salt thereof. In some embodiments the pharmaceutically acceptable salt comprises a potassium salt. Disclosed herein in some embodiments are methods of reducing a level of hemoglobin A1c (HbA1c) in a subject who is prediabetic, the method comprising administering to the subject a therapeutically effective amount of a compound of structural Formula (I)

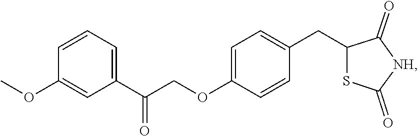 [SMILES: COc1cccc(C(=O)COc2ccc(CC3SC(=O)NC3=O)cc2)c1]

or a pharmaceutically acceptable salt thereof. In some embodiments the pharmaceutically acceptable salt comprises a potassium salt.

In another aspect provided herein, the present disclosure provides methods of inhibiting hepatic mitochondrial pyruvate carrier (MPC) with reduced PPARγ agonism, as compared to pioglitazone, in a subject, the method comprising administering to the subject a therapeutically effective amount of a compound of structural Formula (I)

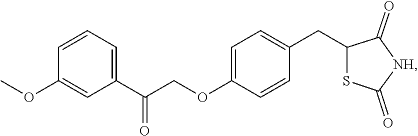 [SMILES: COc1cccc(C(=O)COc2ccc(CC3SC(=O)NC3=O)cc2)c1]

or a pharmaceutically acceptable salt thereof. In some embodiments the pharmaceutically acceptable salt comprises a potassium salt.

In another aspect, the present disclosure provides method for improving a subject's liver's histological features. The methods comprises administering to the subject a therapeutically effective amount of a compound of structural Formula (I)

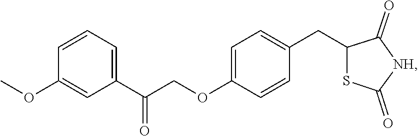 [SMILES: COc1cccc(C(=O)COc2ccc(CC3SC(=O)NC3=O)cc2)c1]

or a pharmaceutically acceptable salt thereof. In some embodiments, the histological features are measured by a NAFLD Activity Score (NAS). The scoring system comprised 14 histological features, 4 of which are evaluated semi-quantitatively: steatosis (0-3), lobular inflammation (0-2), hepatocellular ballooning (0-2), and fibrosis (0-4). Another nine features are recorded as present or absent. A liver biopsy of the subject is formalin-fixed and paraffin-embedded. Serial sections of the liver biopsy are hematoxylin and eosin (H&E) stained. The liver biopsy stained sections are examined for NAS scores. For example, hepatocellular ballooning is graded from 0 to 2 (0: normal hepatocytes with cuboidal shape and pink eosinophilic cytoplasm; 1: presence of clusters of hepatocytes with a rounded shape and pale cytoplasm usually reticulated. Lobular inflammation is defined as a focus of two or more inflammatory cells within the lobule. Foci are counted at 20 magnification (0: none; 1: 2 foci per 20; 2: >2 foci per 20).

As shown in FIG. 17, subjects carrying both copies of the risk alleles of PNPLA3 rs738409-G show NAS histological improvement when treated with MSDC-0602K compound compared to placebo. When subjects are treated with placebo, about 17.6% of the subjects show some NAS histological improvement. When subjects are treated with 62.5 mg of the MSDC-0602K compound, about 17.9% of the subjects show some NAS histological improvement. A significant increase in the number of subjects showing NAS histological improvement occurs when subjects are treated either with 125 mg or 250 mg of the MSDC-0602K compound. About 28.6% of the subjects show some NAS histological improvement when treated with 125 mg of the MSDC-0602K compound and about 34.5% when treated with 250 mg of the MSDC-0602K compound. In some embodiments, the subjects may histological improvement by decreasing NAS by at least 1 score, 2 scores, 3 scores, 4 scores, 5 scores, 6 scores, 7 scores, or 8 scores.

### V. Noninvasive Methods for Assessing NASH Risk

Further, provided here are noninvaseive method for determining whether a subject is at risk for having NASH. The clinical diagnosis of NASH may require findings of hepatic steatosis, lobular inflammation, and ballooning, with or without fibrosis. Even though there seems to be general agreement that liver biopsies are limited for the diagnosis of NASH due to sampling error, intra- and inter-observer variability, significant patient burden, invasiveness, and associated risks, regulatory agencies have required NASH diagnosis by liver biopsy as an entry criterion for NASH studies for approval of new therapies. Some non-invasive tests have been proposed for the diagnosis of NASH. First, imaging techniques such as ultrasound, CT scan, vibration-controlled transient elastography (VCTE) and magnetic resonance imaging (MRI) and MR elastography (MRE) are often used and are mainly directed at detecting steatosis, inflammation or fibrosis. Equally, some combined scores exist for assessment of NASH, such as NAFLD fibrosis score and FIB-4 score, although those scores are mainly validated for detection of fibrosis. Some studies have suggested that liver enzymes and cytokeratin-18 may have some utility in diagnosing NASH; however, research assessing these proposed non-invasive markers, particularly in combination, has been limited.

In some embodiments, the disclosed noninvasive methods comprise assaying an expression level of AST enzyme and an expression level of HbA1c in a subject that is suspected of having NASH. When the subject's AST enzyme level is higher than a reference level of about 27 U/L and the HbA1C level is higher than a reference level of about 6%, the subject has a high probability of having NASH. In some embodiments, the specificity (the ability of the test to correctly identify those without the disease, i.e., true negative rate) of the disclosed methods herein is at least about 70%, 75%, 80%, 85%, 90%, 95%, 95.5%, 96%, 96.6%, 97%, 97.5%, 98%, 98.5%, 99%, or 99.5%. In some embodiments, the specificity of the disclosed methods herein is about 95%. In some embodiments, the specificity of the disclosed methods herein is about 95.4%.

In some embodiments, the expression level of AST is measured from a subject's blood sample. AST is an enzyme found in cells throughout the body but mostly in the heart and liver and, to a lesser extent, in the kidneys and muscles. In healthy individuals, levels of AST in the blood are low. When liver or muscle cells are injured, they release AST into the blood. This makes AST a useful test for detecting or monitoring liver damage. Any suitable methods may be used to measure the AST enzyme levels in the blood samples collected from patients. In some embodiments, the reference level of AST enzyme is at least about 20, 21, 22, 23, 24, 25, 26, 27, 28, 29, or 30 U/L. In some embodiments, the reference level of AST enzyme is at most about 32, 31, 30, 29, 28, 27, 26, 25, 24, 23, 22, 21, or 20 U/L. In some embodiments, the reference level of AST enzyme is about 27 U/L.

In some embodiments, the level of glycated hemoglobin (HbA1c) is measured from a subject's blood sample. Glucose in the subject's bloodstream attaches to the hemoglobin of red blood cells, and the more glucose that is present in the subject's blood, the more it will be attracted to the hemoglobin. Measuring the levels of HbA1c can help determine the average blood glucose control over a 12-week period, as that is the average lifetime of the red blood cell in a human body. In some embodiments, the reference level of HbA1c level is at least about 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, or 7.5%. In some embodiments, the reference level of HbA1c is at most about 7.5%, 7.4%, 7.3%, 7.2%, 7.1%, 7.0%, 6.9%, 6.8%, 6.7%, 6.6%, 6.5%, 6.4%, 6.3%, 6.2%, 6.1%, 6.0%, 5.9%, 5.8%, 5.7%, 5.6%, or 5.5%. In some embodiments, the reference level of HbA1c level is about 42 mmol (6%).

In some embodiments, when the subject's both AST level and HbA1c level are both greater than reference levels disclosed here, the subject is further subject to a noninvasive imaging test. In some embodiments, the imaging tests comprise ultrasound, CT scan, vibration-controlled transient elastography (VCTE), magnetic resonance imaging (MRI), and magnetic resonance elastography (MRE).

### VI. Methods for Evaluating Effectiveness of a Treatment for NASH

In an aspect, provided herein are methods and systems for evaluating an effectiveness of a treatment for non-alcoholic steatohepatitis (NASH). The methods comprise obtaining a baseline level or score of at least three biomarkers selected from the group consisting of Aspartate aminotransferase (AST), Gamma-glutamyltransferase (GGT), Hemoglobin A1c (HbA1c), insulin, Cytokeratin-18 (CK-18), Enhanced Liver Fibrosis (ELF), and alkaline phosphatase in a subject; administering a treatment to the subject for a period of time; obtaining obtaining a treatment level or score of said corresponding at least three biomarkers selected from the group consisting of AST, GGT, HbA1c, insulin, CK-18, ELF, and alkaline phosphatase in said subject after the period of treatment; and generating a composite score based on a difference between said baseline level or score and said treatment level or score, wherein said composite score is indicative of said effectiveness of said treatment.

In some embodiments, the at least three biomarkers are AST, CK-18, and HbA1C. In some embodiments, the at least three biomarkers are AST, GGT, and HbA1C. In some embodiments, the at least three biomarkers are AST, GGT, and insulin. In some embodiments, the at least three biomarkers are AST, GGT, and CK-18. In some embodiments, the at least three biomarkers are AST, GGT, and ELF score. In some embodiments, the at least three biomarkers are GGT, HbA1C, and insulin. In some embodiments, the at least three biomarkers are CK-18, ELF score, and HbA1c. In some embodiments, the at least three biomarkers may be any biomarkers selected from the group consisting of AST, GGT, HbA1c, insulin, CK-18, ELF score, and alkaline phosphatase. Further, in some embodiments, the panel of biomarkers disclosed herein may comprise additional biomarkers. In some embodiments, the panel of biomarkers may contain at least four biomarkers, which may be selected from the group consisting of AST, GGT, HbA1c, insulin, CK-18, ELF score, and alkaline phosphatase. In some embodiments, the panel of biomarkers disclosed herein may comprise at least 5 biomarkers, which may be selected from the group consisting of AST, GGT, HbA1c, insulin, CK-18, ELF score, and alkaline phosphatase. In some embodiments, the panel of biomarkers disclosed herein may comprise at least 6 biomarkers, which may be selected from the group consisting of AST, GGT, HbA1c, insulin, CK-18, ELF score, and alkaline phosphatase. In some embodiments, the panel of biomarkers disclosed herein may comprise at least 7 biomarkers, which may be selected from the group consisting of AST, GGT, HbA1c, insulin, CK-18, ELF score, and alkaline phosphatase.

In some embodiments, baseline levels of AST, GGT, HbA1C, insulin, CK-18, and alkaline phosphatase in a subject before starting any treatment designed to treat or ameliorate NASH or NASH symptoms are obtained. In some cases, a blood sample is obtained from the subject to measure the baseline levels of AST, GGT, HbA1C, insulin, CK-18, and alkaline phosphatase. In some cases, the baseline levels of AST, GGT, HbA1C, insulin, CK-18, and alkaline phosphatase are obtained from published clinical studies. Further, a baseline ELF score is obtained by measuring the levels of tissue inhibitor of metalloproteinases 1 (TIMP-1), amino-terminal propeptide of type III procollagen (PIIINP) and hyaluronic acid (HA) from the blood sample or from published clinical studies.

After the subject receives a treatment, the treatment levels of AST, GGT, HbA1C, insulin, CK-18, and alkaline phosphatase in the subject are obtained. In some cases, a blood sample is obtained from the subject to measure the treatment levels of AST, GGT, HbA1C, insulin, CK-18, and alkaline phosphatase. In some cases, the treatment levels of AST, GGT, HbA1C, insulin, CK-18, and alkaline phosphatase are obtained from published clinical studies.

The differences between the baselines levels of AST, GGT, HbA1C, insulin, CK-18, and alkaline phosphatase and the treatment levels are obtained respectively. Further, the difference between the baseline ELF score and the treatment score is obtained.

In some embodiments, a composite score is obtained comprising the weighted differences of AST, CK-18, and HbA1c levels. The composite score comprises about 40.3% of a difference of AST between a baseline level and a treatment level, about 31.4% of a difference of CK-18 between a baseline level and a treatment level, and about 28.3% of a difference of HbA1c between a baseline level or score and a treatment level or score. In some embodiments, the weighted percentages of the AST, CK-18, and HbA1C differences may vary. The weighted percentage of a difference of AST between a baseline level and a treatment level may be about 30%, 35%, 40%, 40.5%, 41%, 41.5%, 42%, 42.5%, or 43%. The weighted percentage of a difference of CK-18 between a baseline level and a treatment level may be about 25%, 27.5%, 30%, 30.5%, 30.75%, 31%, 31.2%, 31.4%, 31.6%, 31.8%, 40%, 40.5%, or 41%. The weighted percentage of a difference of HbA1C between a baseline level and a treatment level may be about 15%, 18%, 20%, 21%, 22%, 23%, 24%, 25%, 26%, 27%, 27.2%, 27.5%, 27.75%, 28%, 28.2%, 28.4%, 28.6%, 28.8%, 29%, or 30%.

In some embodiments, the period of the treatment may be at least about 2, 4, 6, 8, 12, 18, 24, 36, or 48 months. In some embodiments, the period of the treatment may be at least about 2, 4, 6, 8, 12, 18, 24, 36, 48, or 72 weeks. In some embodiments, the period of treatment may be at least 1, 2, 3, 4, 5, 6, 7, 8, 9, or 10 years.

The term sensitivity, or true positive rate, can refer to a test's ability to identify a condition correctly. For example, in a diagnostic test, the sensitivity of a test is the proportion of patients known to have the disease, who will test positive for it. In some cases, this is calculated by determining the proportion of true positives (i.e. patients who test positive who have the disease) to the total number of individuals in the population with the condition (i.e., the sum of patients who test positive and have the condition and patients who test negative and have the condition).

The quantitative relationship between sensitivity and specificity can change as different diagnostic cut-offs are chosen. This variation can be represented using ROC curves. The x-axis of a ROC curve shows the false-positive rate of an assay, which can be calculated as (1−specificity). The y-axis of a ROC curve reports the sensitivity for an assay. This allows one to easily determine a sensitivity of an assay for a given specificity, and vice versa.

In some embodiments,

### VII. Mechanism of Action

Insulin sensitizers (e.g., pioglitazone) have detrimental side effects due to antagonism of the nuclear transcription factor, PPARγ including, edema, weight gain, and bone loss. Other therapies (e.g., rosiglitazone) with reduced binding affinity to PPARγ, have not been shown to be efficacious as treatment of NASH, particularly with respect to reducing fibrosis in NASH patients.

Aspects disclosed herein provide compounds with significantly reduced ability to bind to PPARγ, while maintaining interaction with the MPC. FIG. 7 shows the relative binding affinities of compound MSDC-0602, the metabolite of MSDC-0602, and the two insulin sensitizers rosiglitazone and pioglitazone with PPARγ. FIG. 7 shows that pioglitazone had a more than ten-fold reduction in binding affinity for PPARγ compared to rosiglitazone, and MSDC-0602 had a more than eight-fold reduction in binding affinity for PPARγ compared to pioglitazone. Moreover, the primary metabolite of MSDC-0602, which comprises more than 90% of the combined exposure, had a more than fifty-fold reduction in binding affinity for PPARγ compared to pioglitazone. In addition to the demonstrable reduction in PPARγ binding, MSDC-0602 and the primary metabolite of MSDC-0602 maintain, and in some cases improve, binding to the MPC, as shown in FIG. 8. Thus, MSDC-0602 is a highly specific modulator of the MPC at concentrations that will not cause meaningful direct activation of PPARγ, resulting in reduced side effects and increased efficacy over the state of the art. The potassium salt, MSDC-0602K, additionally provides improved bioavailability as compared to the free acid alone.

Disclosed herein, in some embodiments are compounds of structural Formula (I), or pharmaceutically acceptable salts thereof that modulate the MPC and mitigate the effects of overnutrition implicated in NASH pathology, insulin resistance, and diabetes. In particular, administration of the compound MSDC-0602K results in a reversal of the effects of carbon delivery to the mitochondria in the form of pyruvate that exceeds energy needs. This modulation of the MPC positions MSDC-0602K upstream in the treatment of the pathophysiology of NASH from other targets in development for NASH. Overnutrition delivers excess pyruvate to the mitochondria through the MPC, driving changes in downstream metabolic pathways through a number of regulatory proteins, and treatment with the MPC modulator, working upstream, is able to reverse these changes.

Table 1 shows the impact of MSDC-0602K relative to overnutrition and overnutrition relative to normal function by tabulating increases and decreases in cell functions and key regulatory proteins.

TABLE 1

| | | | Impact of | Impact of |
|---|---|---|---|---|
| | | | Overnutrition | MSDC-0602K |
| | | | v Normal | vs |
| | Key Factor | Description | Function | Overnutrition |
| Cell | Insulin | Cellular | Decreased | Increased |
| Functions | sensitivity | response | | |
| | | to insulin | | |
| | de novo lipid | Making new | Increased | Decreased |
| | synthesis | fat | | |
| | Fatty acid | Burning Fat | Decreased | Increased |
| | oxidation | | | |
| | Inflammation | Ballooning | Increased | Decreased |
| | | due to injury | | |
| Key | mTORC1 | Important | Increased | Decreased |
| Regulatory | | nutrient sensor | | |
| Proteins | SREBP | Transcription | Increased | Decreased |
| | | factor | | |
| | | regulating fat | | |
| | | synthesis | | |
| | PPARγ, | Transcription | Decreased | Increased |
| | PPARα | factors | | |
| | | regulating fat | | |
| | | metabolism | | |
| | | and | | |
| | | inflammation | | |
| | HIF1α | Transcription | Increased | Decreased |
| | | factor | | |
| | | responding | | |
| | | to oxygen | | |

In some embodiments, compounds of structural Formula (I), or pharmaceutically acceptable salts thereof are effective mediators of the effects of overnutrition. In some embodiments, compounds of structural Formula (I), or pharmaceutically acceptable salts thereof are effective modulators of the mitochondrial pyruvate carrier (MPC).

Aspects disclosed herein provide methods of treating or preventing a disease or condition associated with overnutrition, comprising administering to a subject in need thereof a therapeutically effective amount of a compound as described herein, including embodiments, or the structural Formula (I), or a pharmaceutically acceptable salt thereof. Aspects further disclosed provide methods of treating or preventing NASH, comprising administering to a subject in need thereof a therapeutically effective amount of a compound as described herein, including embodiments, or the structural Formula (I), or a pharmaceutically acceptable salt thereof. Aspects disclosed herein provide methods of treating or preventing diabetes, comprising administering to a subject in need thereof a therapeutically effective amount of a compound as described herein, including embodiments, or the structural Formula (I), or a pharmaceutically acceptable salt thereof. In some embodiments, the pharmaceutically acceptable salt comprises a potassium salt. In some embodiments, diabetes is Type II diabetes.

Aspects disclosed herein provide detecting a presence or a level of one or more biomarkers in a sample obtained from a subject in need thereof. The term “biomarker” as used herein refers to a measurable substance in a subject whose presence and/or level is indicative of a certain phenomenon or phenotype of the subject. A subject in need thereof may be diagnosed with, or suspected of having, a disease or condition (e.g., metabolic inflammation-mediated disease or disorder) disclosed herein. In some instances, a decrease in a level of the one or more biomarkers in a sample obtained from the subject, as compared to a level of the one or more biomarkers (e.g., a “baseline” or “control” level) in a sample obtained from an individual, or group of individuals, that is not diagnosed with the disease or condition, is detected. In some instances, an increase of the one or more biomarkers in a sample obtained from the subject, as compared to a baseline level of the one or more biomarkers in a sample obtained from an individual, or group of individuals, that was not diagnosed with the disease or condition is detected. In some instances, the baseline level of the one or more biomarkers is determined using a single individual, two or more individuals, or a plurality of individuals who were not diagnosed with the disease or condition (e.g., a “normal” individual). In some instances, the sample comprises whole blood, peripheral blood, plasma, serum, urine, saliva, or other biological sample. In some instances, the one or more biomarkers comprises protein, ribonucleic acid (RNA), or deoxyribonucleic acid (DNA), or a combination thereof. In some instances, the one or more biomarkers comprises alanine transaminase (ALT), aspartate aminotransferase (AST), homeostatic model assessment (HOMA) (a method for assessing (3-cell function and insulin resistance (IR) from basal (fasting) glucose and insulin or C-peptide concentrations (HOMA-IR)), and/or hemoglobin lac (HbA1c).

ALT and AST are biomarkers for liver injury and disease. Non-limiting examples of biomarkers of injury include aminotransferases (e.g., ALT and AST), γ-glutamyl transferase (GGT), alkaline phosphatase (ALP), lactate dehydrogenase (LDH), and glutamate dehydrogenase (GLDH). Non-limiting examples of biomarkers of function include prothrombin time, and bilirubin. Non-limiting examples of bio markers of proliferation include α-fetoprotein. A normal range, or baseline, of ALT may be about 6 to about 34 units per liter (U/L) for women and about 6 to about 43 U/L for men. A normal range, or baseline, of AST is about 6 to about 34 U/L for women, and about 9 to about 36 U/L for men. A level of ALT that is above about 34 U/L and 43 U/L for a woman subject and a man subject, respectively, indicates that the subject either has, or will develop, a liver disease or condition (e.g., NASH, NAFLD). A level of AST that is above about 34 U/L and 36 U/L for a woman subject and a man subject, respectively, indicates that the subject either has, or will develop, a liver disease or condition (e.g., NASH, NAFLD).

HbA1c is a biomarker of blood sugar level during the previous two to three months that is used to diagnose and monitor diabetes. Normal adult hemoglobin consists predominantly of HbA (α2β2), HbA2 (α2δ2), and HbF (α2γ2) in the composition of 97%, 2.5%, and 0.5%, respectively. About 6% of total HbA is termed HbA1, which in turn is made up of HbA1a1, HbA1a2, HbA1b, and HbA1c fractions, defined by their electrophoretic and chromatographic properties. HbA1c is the most abundant of these fractions and in health comprises approximately 4-5% of the total HbA fraction. Disclosed herein, in some embodiments, is a normal range, or baseline level, of HbA1c of between 4% and 5.6% in an individual who does not have diabetes. Further provided are levels of HbA1c between 5.7% and 6.4% in a subject that indicate an increased likelihood that the subject will develop diabetes, whereas levels of HbA1c of about 6.5% and higher in a subject indicate that the individual has diabetes.

Aspects disclose herein provide methods of treating a subject in need thereof by administering to the subject a compound as described herein, including structural Formula (I), provided an increase in a level of AST and/or ALT is detected in a sample obtained from the subject, as compared to a level of AST and/or ALT in a normal individual. In some instances, the increase in the level of ALT comprises above about 34 units per liter (U/L) for woman and above about 43 U/L for a man. In some instances, the increase in the level of AST comprises above about 34 U/L for a woman, and above about 36 U/L for a man. In some instances, the subject in need thereof suffers from a metabolic inflammation-mediated disease or disorder. In some instances, the metabolic inflammation-mediated disease or disorder comprises NAFLD, or NASH. In some instances, the subject suffers from fibrosis or fibrostenotic disease. In some instances, the subject suffers from diabetes, including Type II diabetes.

Aspects disclose herein provide methods of treating a subject in need thereof by administering to the subject a compound as described herein, including structural Formula (I), provided an increase in a level of HbA1c is detected in a sample obtained from the subject, as compared to a level of HbA1c in a normal individual. In some instances, the increase in the level of HbA1c comprises above about 6.5%. In some instances, the subject in need thereof suffers from a metabolic inflammation-mediated disease or disorder. In some instances, the metabolic inflammation-mediated disease or disorder comprises NAFLD, or NASH. In some instances, the subject suffers from fibrosis or fibrostenotic disease. In some instances, the subject suffers from diabetes, including Type II diabetes.

Aspects disclosed herein also provide methods of monitoring a progression of a treatment of a subject with the compounds disclosed herein, including structural Formula (I). Also disclosed are methods of optimizing a treatment for a subject with the compounds disclosed herein, including structural Formula (I). In some instances, monitoring and/or optimizing the treatment comprises detecting a level of the one or more biomarkers disclosed herein, including ALT, AST, and/or HbA1c in a sample obtained from the subject. In some instances, the one or more biomarkers comprises protein, RNA, and/or DNA. In some instances, a decrease in the level of ALT, AST, and/or HbA1c in a sample obtained from the subject indicates the treatment of the subject is efficacious.

### VIII. Examples

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

### Example 1. Acute Hepatocyte Mitochondrial Pyruvate Carrier-Dependent and -Independent Effects of MSDC-0602 in Hepatocytes on Insulin Sensitivity and NASH Endpoints in Mice

Insulin-sensitizing thiazolidinediones (TZDs) have shown promise for the treatment of NASH, but their use is limited by side effects of PPARγ-agonism. MSDC-0602 is a “next-generation” TZD which does not bind/activate PPARγ, but binds and modulates the mitochondrial pyruvate carrier (MPC). It has been reported that treatment with MSDC-0602 prevents and reverse liver damage in a mouse model of NASH, and that these beneficial effects require MPC expression in hepatocytes. The purpose of these studies were to differentiate the acute MPC2-dependent and -independent effects of MSDC-0602 by using liver specific MPC2−/− (LS-MPC2−/−) mice. A follow-up study to investigate the circulating exosome miRNA content in a mouse model of NASH with and without MSDC-0602 treatment was then performed.

Methods:

8 week old WT and LS-MPC2−/− mice were fed a diet of 60% fat (Research Diets D12492) for 20 weeks to induce obesity and insulin resistance. Intraperitoneal glucose tolerance tests (GTT) were performed after a 4 h fast by injection of 1 g/kg D-glucose in saline i.p. to assess glucose tolerance. 3 days after the initial GTT, mice were randomized to receive a single gavage of either vehicle (1% CMC, 0.01% Tween-80) or 30 mg/kg MSDC-0602K. Plasma insulin was measured by Singulex assay and plasma ALT levels were measured by commercially-available kits (Teco Diagnostics). qPCR was performed by isolation of total RNA with RNA-Bee and reverse transcription with a high-capacity cDNA synthesis kit (Invitrogen). qPCR was performed on an Applied Biosystems real-time thermocycler.

NASH (HTF-C) Diet Plasma RNA Experiments:

It has been described that hepatocyte-specific MPC2−/− (LS-MPC2−/−) mice are also protected against NASH liver injury, and that these hepatocytes may release exosomes that alter the activation of hepatic stellate cells.

To begin evaluating the exosome cargo of MSDC-0602K-treated mice, WT mice were fed with either a low fat (LF) control diet (Research Diets D09100304) or high trans-fat, fructose, cholesterol (HTF-C) diet composed of 40% trans-fat, 20% fructose, 2% cholesterol (Research Diets D09100301). A subset of mice was fed plain HTF-C diet for 4 or 16 weeks, then switched to HTF-C diet that contained 331 ppm MSDC-0602.

Total RNA was isolated from 400 μL of serum and small RNA sequencing was performed by adapter ligation, cDNA synthesis and size selection of 145-160 base pairs. Sequencing was then performed on an Illumina HiSeq3000.

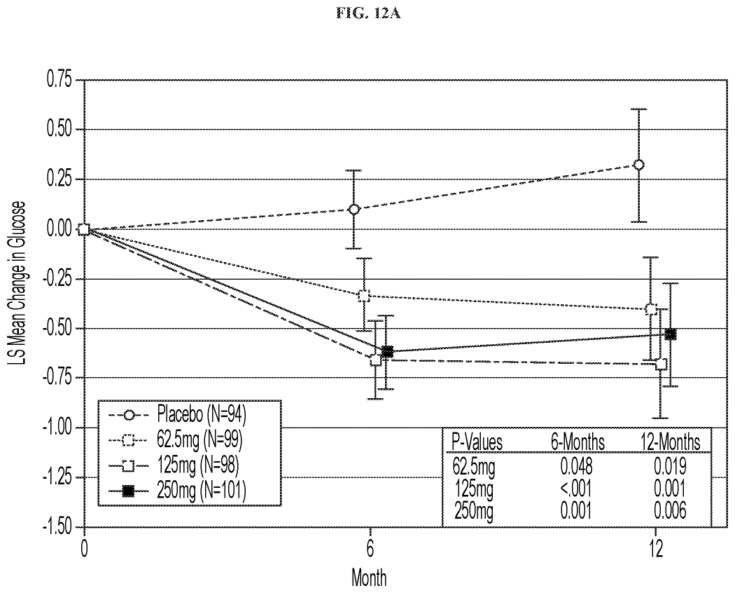

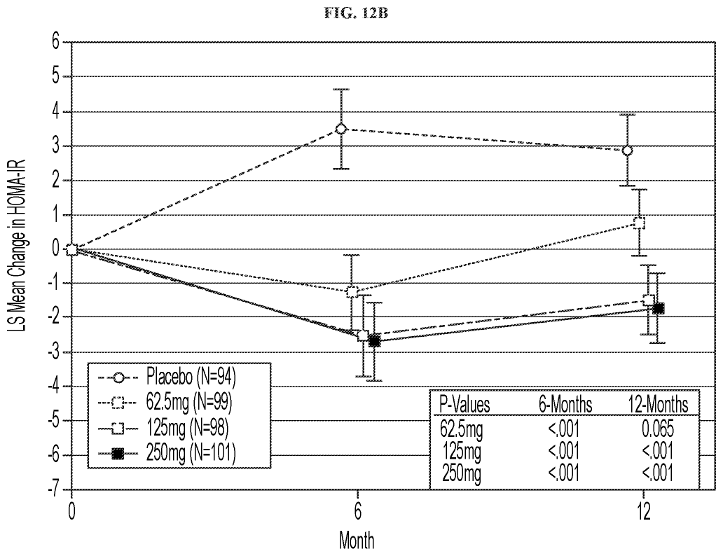

It was found that diet-induced obese LS-MPC2−/− mice display improved glucose tolerance. WT (fl/fl) and LS-MPC2−/− mice were put on 60% HF diet and became equally obese. A GTT reveals that obese LS-MPC2−/− mice are more glucose tolerant. (FIGS. 1A-1C).

Moreover, a single dose of MSDC-0602 improves glucose tolerance. GTT 20 h after a single dose of vehicle or 30 mg/kg MSDC-0602 shows improved glucose tolerance in WT mice treated with MSDC-0602. LS-MPC2−/− again show improved glucose tolerance compared to WT mice, and appear to show no improvement in glucose tolerance. However, plasma insulin values are decreased in both WT and LS-MPC2−/− mice that were treated with MSDC-0602, indicating that both WT and LS-MPC2−/− mice have improved insulin sensitivity after MSDC-0602 treatment. (FIGS. 2A-2C).

Liver-specific MPC2 deletion or acute MSDC-0602 diminish markers of liver injury in NAFLD. LS-MPC2−/− mice, or WT mice treated with a single dose of MSDC-0602 display decreased plasma ALT concentrations (FIG. 3A) and decreased gene expression for hepatic stellate cell activation and fibrotic scar formation (FIG. 3B). For these analyses, LS-MPC2−/− mice appear refractory to the beneficial effects of acute MSDC-0602 treatment.

Altering hepatocyte metabolism by MPC2−/− or MSDC-0602 treatment regulates exosome signaling to hepatic stellate cells as shown in FIG. 4.

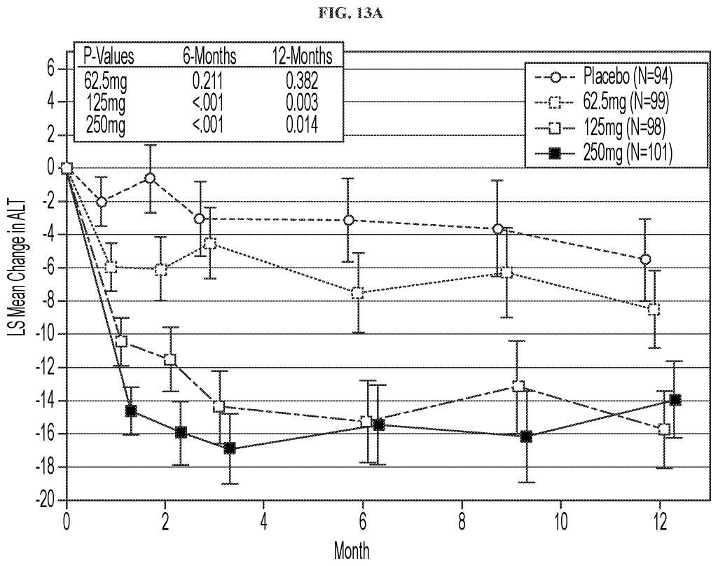

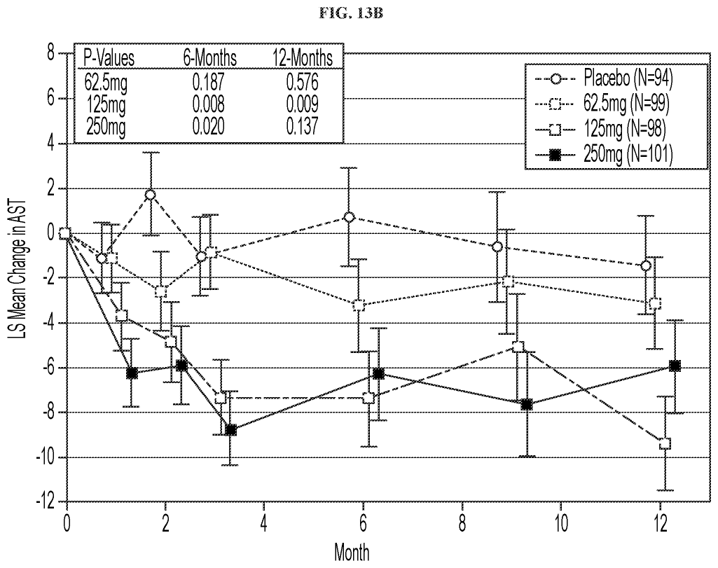

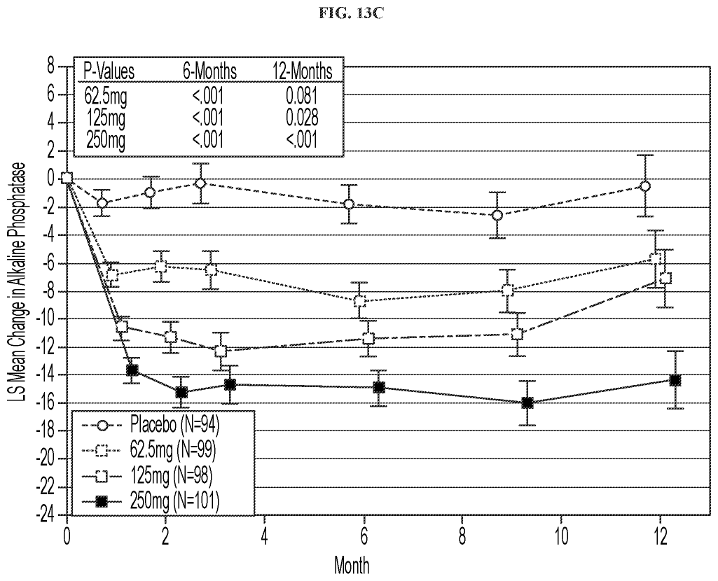

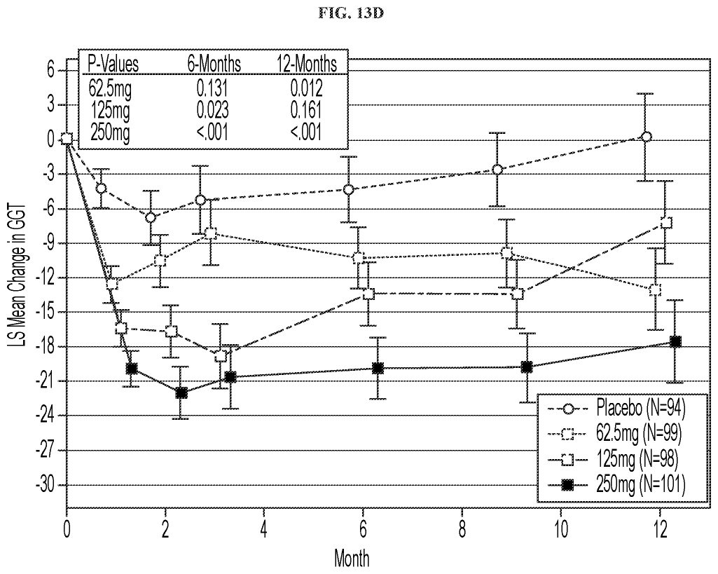

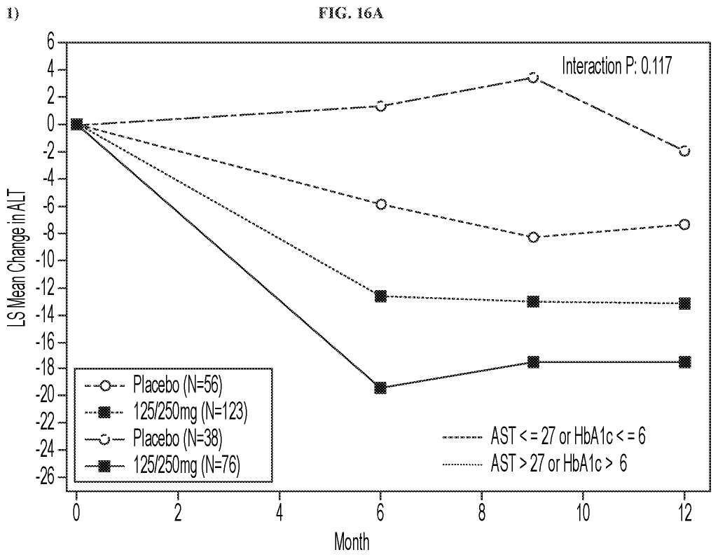

Serum miRNAs are altered in a mouse model of NASH and largely corrected by MSDC0602 treatment. Heat map of serum miRNAs depicts large number of counter-regulated miRNAs with HTF-C diet and treatment with MSDC-0602. Heat map miRNAs (˜230 miRNAs) were selected by filtering data for 2-fold or greater change and FDR<0.1 comparing LF to HTF-C diet (not shown). Examples of miRNAs that are upregulated in NASH and down with MSDC-0602 (FIGS. 13A-13C), down in NASH and up with MSDC-0602 treatment (FIGS. 5D-5F), or simply show large effect of MSDC0602 treatment (FIGS. 5G-5H). Dysregulation of a number of these miRNAs has been previously identified in liver or other fibrotic diseases.

Conclusions:

It was found that liver-specific KO of MPC improves insulin sensitivity in diet-induced obesity. Furthermore, acute dosing with MSDC-0602K improves glucose tolerance, but is not totally dependent on hepatocyte MPC. The major acute effects of MSDC-0602K to attenuate liver injury require hepatocyte MPC and include the release of factors from hepatocytes that affect stellate cells. Altering hepatocyte pyruvate metabolism regulates exosome cargo, which can alter the activation and fibrogenesis of hepatic stellate cells.

### Example 2. Preclinical Studies in NASH

MSDC-0602 was tested in multiple animal models of NASH and has been shown to protect against the high fat, high cholesterol, high sugar diet representative of NASH disease. Mice were fed a diet enriched in trans fat, cholesterol and fructose for 19 weeks to induce hepatic damage, which was assessed by histological changes in the NAS and fibrosis, measured by trichrome staining for collagen. MSDC-0602K (30 mg/kg) was given at either four weeks into the treatment or 16 weeks into the treatment as compared to the vehicle treated mice who were also on the modified diet. The results of mice who were kept on the normal diet are included for comparison. FIG. 10 shows the comparison between mice kept on the normal diet and mice described above. The results show that the setting of overnutrition, signals from hepatocytes can activate stellate cells, which are involved in the initiation of fibrosis. The signals for activation of the stellate cells and fibrosis are attenuated by treatment of the mice or isolated hepatocytes with MSDC-0602K.

### Example 3. Randomized, Double-Blinded Study of Three Doses of MSDC-0602K or Placebo Given Orally Once Daily to Subjects with Biopsy Proven NASH with Fibrosis and No Cirrhosis

As per the current treatment guidelines, a confirmed diagnosis of NASH requires a liver biopsy to determine the NAFLD activity score, or NAS, which assesses the severity of liver damage. The three histology-based components of NAS include steatosis (score range: 0-3), lobular inflammation (score range: 0-3) and hepatocyte ballooning, or damage to liver cells (score range: 0-2), with higher scores indicating the more severe form of the disease. The total NAS score represents the sum of scores for all three components, and ranges from 0-8, with a score of 3-4 indicating borderline NASH, and score of 5-8 occurring in cases largely considered to be diagnostic of NASH. Fibrosis is evaluated separately from NAS on a scale where a score of F3 indicates bridging fibrosis and F4 indicates cirrhosis. Patients who have a composite NAS score of greater than or equal to 4 together with higher levels of fibrosis are most likely to progress toward cirrhosis and complete liver failure as well as hepatocellular cancer.

Detailed Description: This is a randomized, double-blinded study of three doses of MSDC-0602K or placebo given orally once daily to subjects with biopsy proven NASH with fibrosis and no cirrhosis. Visits to the clinic will be at baseline, 1, 2, 3, 6, 9, and 12 months, with one 2-week follow-up visit. Safety will be assessed by monitoring of vital signs, 12 lead electrocardiogram (ECG), physical examinations, safety labs, and adverse events (AEs).

Descriptive Information
A study to evaluate the safety, tolerability & efficacy of MSDC 0602K in patients with NASH.
A phase 2, randomized, double-blind, placebo-controlled, 12-month, multiple-dose study to
evaluate the safety, tolerability and efficacy of three dose levels of MSDC 0602K in patients
with NASH (EMMINENCE ™).

| | |
|---|---|
| Brief | This is a randomized, double-blinded study of three oral doses of |
| Summary | MSDC-0602K or placebo given orally once daily to subjects with |
| | biopsy proven NASH with fibrosis and no cirrhosis. |
| Study Type | Interventional |
| Study Phase | Phase 2 |
| Study Design | Allocation: Randomized |
| | Intervention Model: Parallel Assignment |
| | Masking: Quadruple (Participant, Care Provider, Investigator, |
| | Outcomes Assessor) |
| | Primary Purpose: Treatment |
| Condition | Non-alcoholic Fatty Liver Disease |
| | Non-alcoholic Steatohepatitis |
| | NASH—Nonalcoholic Steatohepatitis |
| | Type II Diabetes |
| | Fibrosis |
| Intervention | Drug: MSDC-0602K |
| | MSDC-0602K capsules |
| | Drug: Placebo |
| | Placebo capsules |
| Study Arms | Active Comparator: MSDC-0602K Dose 1 capsules |
| | MSDC-0602K Dose 1 capsule taken once daily for 360 days |
| | Intervention: Drug: MSDC-0602K |
| | Active Comparator: MSDC-0602K Dose 2 capsules |
| | MSDC-0602K Dose 2 capsules taken once daily for 360 days |
| | Intervention: Drug: MSDC-0602K |
| | Active Comparator: MSDC-0602K Dose 3 capsules |
| | MSDC-0602K Dose 3 capsules taken once daily for 360 days |
| | Intervention: Drug: MSDC-0602K |
| | Placebo Comparator: Placebo capsules |
| | Matching Placebo capsule taken once daily for 360 days |
| | Intervention: Drug: Placebo |
| Eligibility | Selected Inclusion Criteria: |
| Criteria | Adult subjects 18 years of age or greater |
| | Histological evidence of NASH, based on biopsy, with a NAS (NASH |
| | CRN scoring) ≥4 with a score of at least 1 in each component of NAS. |
| | Histological evidence of liver fibrosis defined as NASH CRN System |
| | fibrosis score F1 to F3. |
| | Subjects with type II diabetes mellitus (DM) must be under stable and |
| | reasonable control. |
| | Male and female subjects who are taking Vitamin E should be on a stable |
| | dose of Vitamin E (if ≥400 IU) for a period of at least 3 months prior to |
| | randomization. |
| | Females should be either postmenopausal (at least 12 months since last |
| | menses) or surgically sterilized (bilateral tubal ligation or hysterectomy). |
| | Males with female partners of child-bearing potential must agree to use |
| | adequate contraceptive methods (including a condom, plus one other form |
| | of contraception) if engaging in sexual intercourse. |
| | Willing and able to sign an informed consent document indicating |
| | understanding the purpose of and procedures required for the study and |
| | willingness to participate in the study. |
| | Selected Exclusion Criteria: |
| | Known history of HIV. |
| | Prior liver transplantation. |
| | Other well-documented causes of active chronic liver disease. |
| | History of cirrhosis and/or hepatic decompensation including ascites, |
| | hepatic encephalopathy or variceal bleeding. |
| | History of alcohol abuse or drug abuse within 6 months of Screening. |
| | Type 1 diabetes mellitus. |
| | Current or history of recent (≤6 months) use of ursodeoxycholic acid. |
| | Use of concomitant medications with a known significant metabolism by |
| | CYP2C8 or CPY2C9. |
| | History of diabetic ketoacidosis or hyperosmolar non-ketotic coma within |
| | 6 months prior to randomization. |
| | History of heart failure (including CHF) or previous cardiovascular event |
| | (myocardial infarct, by-pass surgery, or PTCA) within the past 6 months |
| | prior to randomization. |
| | Blood pressure greater than 160/100 mmHg. |
| | Participation in an investigational study or received an investigational drug |
| | within 30 days or 5 half-lives (whichever is longer) prior to study drug |
| | administration. |
| | Malignancy, including leukemia and lymphoma (excluding basal cell and |
| | squamous skin cell cancers and localized prostate cancer) treated within |
| | the last 2 years. |
| Current Primary | Hepatic histological improvement in NAS defined as a decrease of at least |
| Outcome | 2 points with no worsening of fibrosis stage at 12 months. |
| Measures | [Time Frame: 12 months (360 days)] |
| Original | Hepatic histological improvement in NASH defined as a decrease in the |
| Primary | NAS (NASH CRN scoring) by at least 2 points with no concurrent |
| Outcome | worsening of fibrosis at 12 months [Time Frame: 12 months (360 days)] |
| Measures | The reduction in NAS must include either a 1 point reduction in ballooning |
| | or inflammation. Worsening of fibrosis is evaluated using the NASH |
| | Clinical Research Network (CRN) fibrosis staging system and defined as |
| | progression of at least one stage. |
| Current | Proportion of subjects with resolution of NASH with no worsening of |
| Secondary | fibrosis at 12 months. [Time Frame: 12 months (360 days)] |
| Outcome | Proportion of subjects with improvement of fibrosis (CRN staging score) |
| Measures | by at least 1 stage with no worsening of NASH at 12 months. |
| | [Time Frame: 12 months (360 days)] |
| | Mean change from baseline in NAFLD activity score (NAS) and each |
| | one of its components (steatosis, inflammation and ballooning) at 12 |
| | months. [Time Frame: 12 months (360 days)] |
| | Mean change from baseline in fibrosis score at 12 months. |
| | [Time Frame: 12 months (360 days)] |
| Original | Number of participants with treatment-related adverse events as assessed |
| Secondary | by CTCAE v4.0 [Time Frame: 12 months (360 days)] |
| Outcome | Proportion of patients that achieve resolution of NASH by hepatic |
| Measures | histology at 12 months. [Time Frame: 12 months (360 days)] |
| | Resolution of NASH is defined as a ballooning score of 0 and an |
| | inflammation score of 0-1 without worsening of fibrosis. |
| | Proportion of patients that achieve an improvement in liver fibrosis, |
| | determined by a reduction from baseline of at least one point by NASH |
| | CRN scoring at 12 months. [Time Frame: 12 months (360 days)] |
| | Mean changes in the NAFLD activity score and each one of its |
| | components (steatosis, inflammation and ballooning) at 12 months. |
| | [Time Frame: 12 months (360 days)] |
| | Mean changes from baseline in fibrosis score (NASH CRN scoring) at 12 |
| | months. [Time Frame: 12 months (360 days)] |
| Study Update | 402 NASH subjects with fibrosis are fully enrolled. Subjects have (1) |
| | biopsy-confirmed NASH with NAS ≥4, per the NASH Clinical Research |
| | Network, or CRN, scoring system, (2) a score of at least one in each |
| | component of NAS and (3) a fibrosis score of F1 to F3. The study has at |
| | least 50% of subjects who have been diagnosed with Type II diabetes and |
| | at least 50% of subjects who have a fibrosis score of F2 or F3 |
| | Dose 1 Capsules contain a 62.5 mg dosage, Dose 2 Capsules contain a |
| | 125 mg dosage, and Dose 3 Capsules contain a 250 mg dosage |
| | Resolution of NASH is most likely to occur in subjects in whom the |
| | circulating ALT levels are returned to the normal range. Study is planned to |
| | assess preliminary safety, and changes in ALT and AST and other clinically |
| | important biomarkers of liver damage and fibrosis, as well as insulin |
| | sensitivity and glycemic control. |
| | Exploratory endpoints include those that are measured in circulating plasma |
| | or serum. These include non-invasive measures of liver damage and fibrosis, |
| | as well as insulin sensitivity and glucose control. |
| | The relative effectiveness of MSDC-0602K to treat diabetes in the subset of |
| | the subjects with diabetes will be evident from the 6 month changes in |
| | hemoglobin A1c, a reflective measure of blood glucose values over a period |
| | of about 12 weeks. The effects of the treatment on insulin resistance in all |
| | subjects will be measured by changes in the HOMA-IR (a product of |
| | fasting glucose and insulin levels). |
| | The toxicological profiles of the potassium salt MSDC-0602K and its free |
| | acid, have been characterized and no significant safety findings were |
| | observed with doses of MSDC-0602K up to seven-fold the currently used |
| | clinical dose equivalents. |
| | In six- and twelve-month preclinical animal studies with MSDC-0602K, |
| | MSDC-0602K was found to be safe and well-tolerated. Effects similar to |
| | those of other insulin sensitizing agents were observed, such as edema, fluid |
| | volume expansion, body weight, pericardial/thoracic fluid accumulation, |
| | heart weight increase, fatty change in liver, adipose tissue proliferation and |
| | effects in ovary and lymphoid tissue. However, these findings were not |
| | considered clinically meaningful based on the low magnitude of the changes |
| | and lack of adverse physiologic consequences. Chronic studies in rats or |
| | monkeys suggest safety at exposures seven- to eleven-times those in |
| | humans given 250 mg doses of MSDC-0602K (free acid equivalents).] |
| | Doses that have progressed to Phase 2b have been chosen based on the |
| | exposures of parent drug and metabolite (as combined area under the curve, |
| | AUC) in cross-over pharmacokinetic Phase 1 clinical trials and in a Phase 2a |
| | trial in Type II diabetic subjects. The PK is well understood and the half-life |
| | of both parent drug and active metabolite are approximately 11 to 17 hours |
| | and suitable for once-daily oral administration. |
| | Three Phase 1 clinical trials, in a total of 126 healthy volunteers, have been |
| | completed with MSDC-0602 or MSDC-0602K. Most commonly reported |
| | adverse events with MSDC-0602 were headache, emesis, diarrhea and |
| | abdominal bloating. No deaths, serious adverse events, severe adverse |
| | events or discontinuations due to adverse events were reported. Overall, it |
| | was determined that there were no safety laboratory tests, vital signs, |
| | physical examination findings or electrocardiogram abnormalities that were |
| | considered clinically significant. In addition, a randomized, placebo |
| | controlled Phase 2a study was conducted in 129 subjects with Type II |
| | diabetes to compare the pharmacokinetic, safety and efficacy profiles of |
| | three doses of MSDC-0602 against 45 mg of pioglitazone to inform |
| | exposures for future trials. Crossover studies of MSDC-0602K with these |
| | free acid tablets were then used to select the doses to be tested in our |
| | Phase 2b trial in referenced above. |

### Example 4. Addition of MSDC-0602K to Cell Types with Fibrosis

High concentrations of sugars and fatty acid in tissue culture medium can activate stellate cells in 3-dimensional bioprinted human liver organoids containing a mixture of cell types and that this results in fibrosis as measured by staining for collagen. The addition of MSDC-0602K to the tissue medium both prevented and reversed the effects of the nutrients.

Although the disclosure has been described with reference to the above examples, it will be understood that modifications and variations are encompassed within the spirit and scope of the disclosure. Accordingly, the disclosure is limited only by the following claims.

### Example 5. Interim Analysis of the EMMINENCE Phase 2b Clinical Trial

An interim analysis of the EMMINENCE Phase 2b trial was conducted in the first 328 subjects who reached their six-month follow-up visit. The interim analysis of explanatory endpoints showed statistically significant reductions in liver enzymes, including ALT and AST, measured at six months compared to baseline. In the two highest dose groups, at least 50% of patients with elevated baseline Alanine transaminase (ALT) or Aspartate Aminotransferase (AST) improved into the normal range at six months, as shown in Table 3. Statistically significant reductions in homeostatic model assessment (HOMA), a method for assessing (3-cell function and insulin resistance (IR) from basal (fasting) glucose and insulin or C-peptide concentrations (HOMA-IR) and Hemoglobin lac (HbA1c) were also observed in all MSDC-0602K dose cohorts. Overall adverse event rates were similar across placebo and all doses of MSDC-0602K.

The subjects included in this interim analysis had significant liver disease, as established by liver biopsy, with an average NAS at baseline of 5.3. Almost 60% of these subjects had a baseline fibrosis score of F2 or F3 and approximately 50% also had a diagnosis of Type II diabetes at baseline. Overall, baseline characteristics were well-balanced across treatment groups. Table 2 provides baseline characteristics of subjects included in the interim analysis.

TABLE 2
Baseline Characteristics of Subjects Included in Interim Analysis

| | | MSDC-0602K | | |
|---|---|---|---|---|
| All Subjects | Placebo | 62.5 mg | 125 mg | 250 mg |
| (n = 328) | (n = 78) | (n = 81) | (n = 84) | (n = 85) |
| Age (mean, years) | 54.4 | 56.7 | 56.3 | 57.5 |
| Male | 47.4% | 39.5% | 35.7% | 42.4% |
| Female | 52.6% | 60.5% | 64.3% | 57.6% |
| Weight (mean, kg) | 102.6 | 96.2 | 99.2 | 98.2 |
| Type 2 Diabetes | 50.0% | 53.1% | 52.4% | 51.8% |
| Present | | | | |
| ALT (mean, U/L) | 59.3 | 58.6 | 49.8 | 58.0 |
| AST (mean, U/L) | 42.4 | 45.2 | 42.7 | 44.8 |
| Diabetic Subjects | Placebo | 62.5 mg | 125 mg | 250 mg |
| (n = 170): | (n = 39) | (n = 43) | (n = 43) | (n = 43) |
| HbA1c | 6.83% | 7.09% | 6.90% | 6.97% |

FIGS. 9A-9B show the levels of change from baseline in ALT (FIG. 9A) and AST (FIG. 9B) by visit over 6 months of treatment with MSDC-0602K (62.5 mg, 125 mg, 250 mg). Statistically significant placebo-corrected reductions at six months in ALT levels were observed in the 125 mg and 250 mg dose cohorts (FIG. 9A). A statistically significant placebo-corrected reduction at six months in AST levels was observed in the 125 mg dose cohort (FIG. 9B). Placebo-corrected reductions at six months of 14.3 Units/Liter, U/L, (p<0.001) and 7.9 U/L (p=0.012) in ALT and AST, respectively, in the 125 mg cohort, and 10.6 U/L (p=0.004) and 4.0 (not significant) in ALT and AST, respectively, in the 250 mg cohort. Placebo-corrected reductions, relative to baseline, were 25% and 18% in ALT and AST, respectively, in the 125 mg cohort, and 19% and 9% in ALT and AST, respectively, in the 250 mg cohort. Importantly, normalization of ALT and AST was observed across all three dose levels of MSDC-0602K, with the two highest dose groups having at least 50% of subjects improved to normal range. ALT normal range is defined as 6 to 34 U/L and 6 to 43 U/L for women and men, respectively, and AST normal range is defined as 9 to 34 U/L and 11 to 36 U/L for women and men, respectively. Table 3 shows the percentage of patients with high baseline values who returned to normal values.

TABLE 3
Percentage of Patients with High Baseline ALT and
AST Values who Returned to Normal Range

| | | MSDC-0602K | | |
|---|---|---|---|---|
| | Placebo | 62.5 mg | 125 mg | 250 mg |
| ALT | 15% | 29% | 60% | 55% |
| AST | 20% | 36% | 50% | 52% |

Improvements were also observed in bilirubin, alkaline phosphatase, and gamma GT liver enzymes. Across all subjects, statistically significant improvements were observed at six months in fasting glucose, HbA1c (Table 4), insulin levels, and HOMA-IR at 125 mg and 250 mg doses of MSDC-0602K. Among Type II diabetes subjects, for whom mean baseline values for HbA1c were relatively low compared to populations in clinical trials of other Type II diabetes therapies, statistically significant improvement was observed in HbA1c at all doses of MSDC-0602K. Adiponectin increased significantly in all treatment cohorts in a dose dependent manner, and there was a decrease in C-Reactive Protein levels seen in the two lowest doses. Table 4 shows HbA1c measurements in subject with Type II Diabetes at baseline and six months. Also observed were trends for improvement in additional biomarkers of fibrosis such as hematocrit and, overall, a neutral effect on lipids.

TABLE 4
Mean HbA1c Measurements in Subjects with Type II
Diabetes at Baseline and Six Months

| | | MSDC-0602K | | |
|---|---|---|---|---|
| | Placebo | 62.5 mg | 125 mg | 250 mg |
| Baseline | 6.83% | 7.09% | 6.90% | 6.97% |
| Placebo-corrected | | ▾0.37 | ▾0.55 | ▾0.45 |
| change from Baseline | | | | |
| at 6 months | | | | |
| p-value | | 0.022 | 0.001 | 0.006 |

The overall rates of treatment emergent adverse events were similar across placebo and all MSDC-0602K cohorts. Importantly, the frequency of any degree of peripheral edema, a finding associated with direct PPARγ agonism, observed at six months was similar to that observed at baseline and was comparable across placebo and MSDC-0602K cohorts. Table 5 provides peripheral edema present at baseline and at six months.

TABLE 5
Peripheral Edema Present at Baseline and Six Months

| | | MSDC-0602K | | | |
|---|---|---|---|---|---|
| | Placebo | 62.5 mg | 125 mg | 250 mg | Total |
| | (n = 78) | (n = 81) | (n = 84) | (n = 85) | (n = 328) |
| Baseline | 7.70% | 13.60% | 13.10% | 9.40% | 11.00% |
| 6 months | 12.50% | 7.80% | 13.50% | 11.10% | 11.20% |

To preserve the blinding of this ongoing clinical trial, adverse events that were reported by fewer than five subjects in an individual cohort were not unblinded as to distribution across cohorts. Treatment emergent serious adverse events were reported in fourteen subjects, none of which were determined by the investigator to be drug-related.

### Example 6: Analysis of the EMMINENCE Phase 2b Clinical Trial at 12 Months

Study Design: EMMINENCE was a randomized, double-blind evaluation of three oral daily doses of MSDC-0602K (62.5 mg, 125 mg and 250 mg) or placebo given for 12 months to adult patients with biopsy-proven NASH with fibrosis and no cirrhosis. The primary efficacy endpoint was assessed at 12 months. Clinic visits were conducted at baseline, 1, 2, 3, 6, 9, and 12 months, followed 2 weeks later by a safety visit. An interim analysis of safety and efficacy not related to the primary and secondary endpoints was conducted when 6 month data were available for 328 patients (see Example 5, above.) Interim results were provided only as summary statistics to avoid any unmasking of the study team or sponsor.

Local principal investigators evaluated patients at each visit, and determined the adjustment of concomitant anti-diabetes medications and ancillary therapeutics based on their examinations. They remained masked to the treatment assignment throughout the study. Safety was overseen by the Sponsor's Medical Monitor. The study was conducted in accordance with ICH Good Clinical Practice and all applicable regulatory requirements. Patients provided written informed consent prior to study participation. The protocol and consent forms were approved by relevant institutional review boards.

Participants: Eligible patients were at least 18 years old with NASH (NAS≥4; score ≥1 in each component [steatosis, ballooning, inflammation]) with F1 to F3 fibrosis on liver biopsy taken between 9 months prior to screening and randomization according to a central histopathologist. Detailed inclusion and exclusion criteria for the study are given in Table 7.

TABLE 7
EMMINENCE study eligibility criteria

| Inclusion criteria | Exclusion criteria |
|---|---|
| 1. Adult subjects at least 18 years of age. | 1. Known history of HIV. |
| 2. Histological evidence of NASH, based on | 2. Prior liver transplantation. |
| biopsy, with a NAS (NASH CRN scoring) ≥4 with | 3. Other well-documented causes of active chronic |
| a score of at least 1 in each component of NAS. | liver disease, including, but not restricted to: |
| 3. Histological evidence of liver fibrosis defined | history of positive hepatitis B surface antigen |
| as NASH CRN System fibrosis score of F1 to F3. | (HBsAg) or HCV RNA (subjects must be viremia |
| 4. Qualifying liver biopsy must be within 9 months | free fora minimum of 2 years), suspicion of drug- |
| prior to or during Screening. If no usable liver | induced liver disease, alcoholic liver disease and |
| biopsy is available within 9 months prior to | auto-immune hepatitis, Wilson’s disease |
| Screening, a liver biopsy must be performed | hemochromatosis, primary biliary cirrhosis, |
| during Screening after the following criteria are | primary sclerosing cholangitis, genetic |
| confirmed: | hemochromatosis, known |
| a. AST ≥20 U/L | or suspected HCC, |
| b. FibroScan with CAP score ≥270 db/m and | history of planned liver transplant or current MELD |
| kPa ≥8.5 | score >12. |
| 5. Subjects with type 2 diabetes mellitus (DM) | 4. History of cirrhosis and/or hepatic |
| must be under stable and reasonable control, | decompensation including ascites, hepatic |
| defined as under a healthcare provider’s care for | encephalopathy or variceal bleeding. Platelet |
| DM with glycosylated hemoglobin [HbA1c] | count <100,000/μL of blood at Screening. |
| ≤9.5%. Subjects taking permitted anti-diabetic | 5. AST or ALT >5 times the upper limit of normal |
| therapies (metformin, | at Screening. |
| GLP-1 agonists, SGLT2 | 6. Total bilirubin >1.8 mg/dL |
| inhibitors, sulfonylureas | unless diagnosis of |
| or DPP4 inhibitors) must | Gilbert’s disease or serum |
| be on stable doses for | albumin <2.5 gms/dL |
| a period of at least 3 | at Screening. |
| months prior to randomization. 6. Subjects taking | 7. INR ≥1.5 times ULN at Screening or other evidence |
| Vitamin E must be on a stable | of impaired coagulation. |
| dose (if ≥400 IU) for a period of at least 3 | 8. History of alcohol abuse or drug abuse within 6 |
| months prior to randomization. | months of Screening. |
| 7. Females must be either postmenopausal (at | 9. Inability to safely obtain a liver biopsy. |
| least 12 months since last menses) or surgically | 10. Current or history of Type 1 diabetes mellitus. |
| sterilized (bilateral tubal ligation, bilateral | 11. Current or history of recent (≤6 months) use |
| oopherectomy and/or | of ursodeoxycholic acid. |
| hysterectomy) verified by a | 12. Current use of any diabetes |
| follicle-stimulating hormone | medications other |
| (FSH) test result ≥40 | than metformin, GLP-1 |
| mlU/mL. Those with FSH | agonists, SGLT2 inhibitors, |
| <40 or with bilateral | sulfonylureas or DPP4 |
| tubal ligation must use a | inhibitors three months |
| barrier method of birth | prior to the baseline diagnostic |
| control. In addition, all | liver biopsy or |
| females must have a | during the study unless |
| negative pregnancy test at | as specified in the |
| Screening and Day 1 regardless | protocol. Current use of |
| of childbearing potential. For | insulin or PPARy agonists |
| postmenopausal women only, if FSH levels are | (pioglitazone or rosiglitazone) is not allowed for |
| above 40 mlU/mL and serum pregnancy results | the duration of the study. 13. Use of concomitant |
| are indeterminant, the | medications with a known |
| subject will be assessed | significant metabolism by |
| as not pregnant. Males | CYP2C8 or CYP2C9 |
| with female partners of | including paclitaxel, |
| child-bearing potential | phenytoin, warfarin, celecoxib, |
| must agree to use | tolbutamide, or replaglinide |
| adequate contraceptive | for the duration of the study. |
| methods (including a condom, plus one other | 14. History of diabetic ketoacidosis or |
| form of contraception) if | hyperosmolar non-ketotic |
| engaging in sexual intercourse. | coma within the 6 |
| 8. Body Mass Index (BMI) < 50 kg/m². | months prior to randomization. |
| 9. Willing and able to sign an informed consent | 15. History of heart failure (including CHF) or |
| document indicating understanding of the | previous cardiovascular event (acute coronary |
| purpose of and procedures required for the study | syndrome, myocardial infarction, coronary by-pass |
| and willingness to participate in the study. | surgery, or catheter based coronary intervention) |
| 10. Willing and able to attend all study visits and | within the 6 months prior to randomization. |
| undergo all protocol- specified procedures | 16. Serum creatinine >2 mg/dL at Screening. |
| including liver biopsies at baseline and at the end | 17. Current or recurrent disease that may affect |
| of study treatment (one year). | the action, absorption or disposition of the study |
| | treatment, or clinical or laboratory assessments. |
| | 18. Current or history of severe or unstable |
| | disorder (medical or psychiatric) requiring |
| | treatment that may make the subject unlikely to |
| | complete the study. |
| | 19. Blood pressure greater than 160/100 mmHg. |
| | Subjects with elevated BP (but <160/100 mmHg) |
| | with or without current treatment are allowed at |
| | the discretion of the Investigator and primary care |
| | physician. Individuals with hypertension must have |
| | been stabilized to the current treatment regimen |
| | for at least 6 weeks prior to randomization. |
| | 20. Known or suspected intolerance or |
| | hypersensitivity to the study drugs, closely related |
| | compounds or any of their stated ingredients. |
| | 21. Participation in an investigational study (other |
| | than a non-treatment registry study) or received |
| | an investigational drug within 30 days or 5 half- |
| | lives (whichever is longer) prior to randomization. |
| | 22. Blood donation of 1 unit or more within 56 |
| | days of randomization. |
| | 23. Plasmapheresis or plasma donation within 30 |
| | days of randomization. |
| | 24. Single 12-lead ECG demonstrating a |
| | screening QTc >450 msec in men and >470 |
| | msec in women. A single repeat ECG may be |
| | done at the Investigator’s discretion. |
| | 25. Any surgical or medical condition which may |
| | significantly alter the absorption of the study drug |
| | including, but not limited to the following: history of |
| | major gastrointestinal tract surgery such as |
| | gastrectomy, gastroenterostomy, bowel resection, |
| | gastric bypass, gastric stapling, or gastric banding, |
| | currently active inflammatory bowel syndrome. |
| | Subjects with reversal of gastric sleeve or banding |
| | are allowed. |
| | 26. Evidence of clinically relevant pathology that |
| | could interfere with the study results or put the |
| | patient’s safety at risk. |
| | 27. Malignancy, including leukemia and lymphoma |
| | (excluding basal cell and squamous cell skin |
| | cancers and localized prostate cancer) not treated |
| | within the last 2 years. |

Randomization and Masking: Eligible patients were randomized according to a central scheme 1:1:1:1 across the 4 groups within strata defined by presence of T2D (Yes/No), use of Vitamin E≥400 IU (Yes/No), and fibrosis score (F1 or ≥F2). Medication was supplied as bottles of masked capsules. Patients, investigators, the sponsor and all other parties except a sequestered statistical consultant were masked to treatment assignment throughout the conduct of the study and a formal statistical analysis plan was finalized before the statistical consultant unmasked any other parties.

Procedures: Patients had a liver biopsy at screening/baseline and at 12 months. If no usable liver biopsy was available within 9 months prior to screening, a percutaneous liver biopsy was performed in patients with AST≥20 U/L and FibroScan CAP score ≥270 db/m and kPa≥8.5. Digitized slides were read by a single expert histopathologist according to NASH Clinical Research Network criteria. Baseline biopsies were read first for study qualification and a second time mixed randomly among 12-month biopsies. Fasting blood samples for routine central laboratory analyses were collected at each visit, and frozen samples collected at selected visits for biomarker assays.

Efficacy and Safety Measures

Efficacy endpoints: The primary efficacy endpoint was hepatic histological improvement in NAS defined as ≥2-point NAS decrease with a ≥1-point reduction in either ballooning or lobular inflammation and no increase in fibrosis stage at 12 months. Secondary efficacy endpoints included (1) NASH resolution defined as hepatocellular ballooning score of 0 and lobular inflammation score of 0-1 with no increase in fibrosis stage at 12 months, (2) fibrosis improvement defined as ≥1-stage decrease in fibrosis with no worsening of lobular inflammation or hepatocellular ballooning, (3) change from baseline in NAFLD activity score (NAS) and each one of its components (steatosis, lobular inflammation and ballooning) at 12 months, and (4) change from baseline in fibrosis score at 12 months. Exploratory efficacy endpoints included shifts in the central histopathologist's NASH diagnosis confirmation, and changes from baseline to 12 months in markers of glycemic control and insulin sensitivity, liver function/injury, and apoptosis and liver fibrosis.

Safety: Safety was evaluated by adverse event incidence, and changes in clinical laboratory values, vital signs, and ECG parameters. Major adverse cardiovascular events (MACE) including death, non-fatal myocardial infarction (MI), unstable angina (USA) hospitalization, hospital admission for heart failure (HF), or non-fatal ischemic stroke; liver events including ascites, hepatic encephalopathy, variceal hemorrhage, hepatocellular carcinoma (HCC), or liver transplant; severe hypoglycemia; and bone fracture requiring surgery or hospitalization were adjudicated by an independent physician.

Statistical Methods:

Each of the three MSDC-0602K dose groups were compared to the placebo group at the two-sided 0.05 significance level. It was estimated that 85 patients per arm would provide approximately 80% power to detect a difference in response rates of 39.5% in any MSDC-0602K dose group versus 20% in the placebo group. As this was a phase 2b dose-ranging study, no multiplicity adjustment was employed.

Using a modified intent-to-treat approach, treated patients were included for analysis in their randomly-assigned treatment groups. Participation of sites with serious GCP or quality concerns were terminated and their data excluded from the analyses. Post-baseline measures through 14 days after the last study drug dose were included. No errors in treatment assignment occurred. The central histopathologist's re-read of baseline biopsies was used as baseline. Patients who no longer qualified on re-read were included in the analyses. Each active treatment group was compared to placebo with respect to the primary and secondary histopathologic endpoints using logistic regression including adjustment for randomization strata (diabetes mellitus, baseline fibrosis F1 versus F2/F3), and ordinal baseline NAS. Stratification-adjusted risk differences adjusted similarly are provided. The primary analyses included only observed data; sensitivity analyses included multiply-imputing missing follow-up and considering missing follow-up as a non-responder. Each active dose was compared to placebo regarding changes in NASH CRN scores using analysis of covariance with adjustment for age, sex, diabetes mellitus, Vitamin E≥400 IU, baseline fibrosis F1 versus F2/F3, and the baseline value. Changes in laboratory markers from baseline to 6 months and to 12 months were analyzed similarly.

Post-hoc analyses: Each active dose was compared to placebo regarding the proportion where the pathologist's NASH diagnosis shifted from confirmed at baseline (re-read) to not confirmed at month 12 using logistic regression. Analyses examining results in patients with more severe liver disease (AST>27 U/L) and higher baseline HbA1c (HbA1c>6.0% or >7.0%) were conducted. The AST threshold was selected based on evidence suggesting this value may differentiate NAFLD from NASH in patients at risk, and was also the first quartile baseline value in EMMINENCE. HgA1C≥6% is an accepted threshold for inclusion into many T2D studies and was the median baseline value in EMMINENCE and HbA1c>7% is a value accepted as the threshold for poorly controlled T2D. Tests of whether the effects of the combined 125 and 250 mg MSDC-0602K doses versus placebo differed in patients in the subgroup (interaction tests) were constructed by contrasting the least square mean treatment differences over all time points in the two groups for changes in laboratory markers and through a treatment-by-subgroup interaction term in the logistic regression model for binary endpoints. Because similar effects on liver function and glycemic control were observed in the two highest MSDC-0602K doses (125 and 25 mg), the combined 125 and 250 mg MSDC-0602K doses were compared with placebo regarding key primary, secondary and exploratory endpoints. Analyses were conducted to gauge MSDC-0602K effects across multiple biomarkers of apoptosis and liver fibrosis (APRI, Fib-4, FibroTest, ELF, and CK-18). Changes in each biomarker were transformed to z-scores by subtracting from each patient's value the overall mean and dividing by the overall standard deviation. Groups were compared with respect to the average of a patient's z-scores over the biomarkers at 6 months and at 12 months comparing the combined 125 and 250 mg doses against placebo, using Kruskal-Wallis tests. Because APRI and Fib-4 are highly correlated being derived from two common variables, AST and platelet count, the average z-score excluding APRI was also examined. Finally, because MSDC-0602K treatment was associated with weight gain, we examined the association of changes in insulin, HbA1c and AST with changes in weight in pateints treated by placebo or study drug.

Results

Between September 2016 and May 2018, 1090 patients were screened and 402 randomized in 57 United States centers; all patients were treated with study medication. Ten randomized patients in five prematurely terminated centers were excluded before the interim analysis due to major quality concerns. The participant flow is presented in FIG. 11.

Baseline characteristics and concomitant therapies in the patients enrolled in the study were generally well balanced between the groups (Table 8). Mean age was 56.1±10.7 years. Average weight was 99.7±19.9 kg; 52.3% had T2D, 4.8% previous coronary artery disease, 36.5% hyperlipidemia and 8.4% peripheral edema at baseline. At inclusion, about half (49.7%) the patients received anti-diabetes therapy, most of whom (36.2%) were treated with metformin. Almost half (48.7%) the patients received lipid modifying agents.

TABLE 8
Baseline characteristics and concomitant medications at study entry of patients
randomized in the EMMINENCE study

| | | MSDC- | MSDC- | MSDC- | |
|---|---|---|---|---|---|
| | | 0602K | 0602K | 0602K | |
| | Placebo | 62.5 mg | 125 mg | 250 mg | Total |
| Disease | (N = 94) | (N = 99) | (N = 98) | (N = 101) | (N = 392) |
| Age, y (Mean [SD]) | 54.6 [11.21] | 56.9 [10.28] | 56.0 [10.89] | 56.8 [10.42] | 56.1 [10.70] |
| Sex (M:F) | 43:51 | 43:56 | 35:63 | 43:58 | 164:228 |
| Race (Black or | 5:85 | 3:93 | 3:91 | 3:91 | 14:360 |
| African American: | | | | | |
| White) | | | | | |
| Ethnicity (Hispanic or | 25:69 | 32:67 | 29:69 | 33:68 | 119:273 |
| Latino:Not Hispanic | | | | | |
| or Latino) | | | | | |
| Weight, kg (Mean | 102.08 | 98.93 | 99.95 | 98 [18.871] | 99.7 [19.922] |
| [SD]) | [21.527] | [20.185] | [19.175] | | |
| BMI, kg/m² (Mean | 35.03 [5.574] | 34.68 [5.177] | 35.93 [6.121] | 35.03 [6.118] | 35.16 [5.761] |
| [SD]) | | | | | |
| Waist | 113.52 | 112.86 | 114.09 | 112.89 | 113.33 |
| Circumference, cm | [15.066] | [12.466] | [14.025] | [13.486] | [13.726] |
| (Mean [SD]) | | | | | |
| T2D | 50.0 | 55.6 | 52.0 | 51.5 | 52.3 |
| Cardiac disorders | 18.1 | 28.3 | 19.4 | 10.9 | 19.1 |
| Atrial fibrillation | 1.1 | 4.0 | 1.0 | 1.0 | 1.8 |
| Coronary artery | 5.3 | 7.1 | 5.1 | 2.0 | 4.8 |
| disease | | | | | |
| Hypothyroidism | 20.2 | 22.2 | 23.5 | 17.8 | 20.9 |
| Oedma peripheral | 5.3 | 9.1 | 11.2 | 7.9 | 8.4 |
| Hepatobiliary | 79.8 | 83.8 | 76.5 | 77.2 | 79.3 |
| disorders | | | | | |
| Cholelithiasis | 18.1 | 10.1 | 18.4 | 13.9 | 15.1 |
| Hepatic steatosis | 43.6 | 46.5 | 36.7 | 40.6 | 41.8 |
| Hepatitis A | 1.1 | 2.0 | 4.1 | 2.0 | 2.3 |
| Hepatitis B | 1.1 | 0.0 | 1.0 | 1.0 | 0.8 |
| Hepatitis C | 0.0 | 3.0 | 1.0 | 4.0 | 2.0 |
| Hyperlipidaemia | 36.2 | 38.4 | 33.7 | 37.6 | 36.5 |
| Diabetic neuropathy | 3.2 | 4.0 | 1.0 | 1.0 | 2.3 |
| Depression | 24.5 | 25.3 | 24.5 | 33.7 | 27.0 |
| Chronic kidney | 2.1 | 0.0 | 1.0 | 1.0 | 1.0 |
| disease | | | | | |
| Asthma | 16.0 | 11.1 | 12.2 | 11.9 | 12.8 |
| Chronic obstructive | 4.3 | 1.0 | 6.1 | 3.0 | 3.6 |
| pulmonary disease | | | | | |
| Sleep apnoea | 22.3 | 22.2 | 18.4 | 21.8 | 21.2 |
| syndrome | | | | | |
| Alcohol use | 5.3 | 2.0 | 3.1 | 5.0 | 3.8 |
| Substance use | 1.1 | 2.0 | 2.0 | 1.0 | 1.5 |
| Hypertension | 72.3 | 68.7 | 67.3 | 59.4 | 66.8 |
| RAAS blockers | 49 (52.1) | 43 (43.4) | 46 (46.9) | 43 (42.6) | 181 (46.2) |
| Aspirin | 32 (34.0) | 36 (36.4) | 29 (29.6) | 23 (22.8) | 120 (30.6) |
| Beta blockers | 20 (21.3) | 22 (22.2) | 24 (24.5) | 19 (18.8) | 85 (21.7) |
| Calcium blockers | 20 (21.3) | 15 (15.2) | 17 (17.3) | 12 (11.9) | 64 (16.3) |
| Steroids | 4 (4.3) | 5 (5.1) | 5 (5.1) | 4 (4.0) | 18 (4.6) |
| Diuretics | 24 (25.5) | 20 (20.2) | 22 (22.4) | 18 (17.8) | 84 (21.4) |
| Drugs for COPD | 22 (23.4) | 14 (14.1) | 22 (22.4) | 15 (14.9) | 73 (18.6) |
| Anti-diabetics | 47 (50.0) | 49 (49.5) | 50 (51.0) | 49 (48.5) | 195 (49.7) |
| Metformin | 36 (38.3) | 36 (36.4) | 36 (36.7) | 34 (33.7) | 142 (36.2) |
| Lipid modifying | 48 (51.1) | 53 (53.5) | 45 (45.9) | 45 (44.6) | 191 (48.7) |
| agents | | | | | |
| Thyroid therapy | 17 (18.1) | 22 (22.2) | 28 (28.6) | 19 (18.8) | 86 (21.9) |

Histological Response

Although results are suggestive of a histologic dose response, no statistically significant difference between any MSDC-0602K dose and placebo with respect to primary and secondary binary histologic endpoints was observed (Table 9). The primary endpoint, NAS improvement, occurred in 29.7, 29.8, 32.9, and 39.5% of patients in the placebo and 62.5, 125 and 250 mg MSDC-0602K groups, respectively. Results were similar for sensitivity analyses imputing for missing follow-ups. The adjusted mean difference in NAS change to 12 months was statistically significantly greater in the MSDC-0602K 125 (−0.6 points) and 250 (−0.5 points) mg groups than in placebo, with statistically significantly greater changes in steatosis in these dose groups than in placebo (−0.2 and −0.3 points, respectively). Twelve-month fibrosis stage changes did not differ significantly between placebo and any active dose group. A confirmed NASH diagnosis at baseline (re-read) was not confirmed at 12 months (i.e., improved) in 20.3, 32.1, 26.5, and 30.2% of patients in placebo, and 62.5, 125, and 250 mg MSDC-0602K groups, respectively, with no statistically significant differences from placebo (Table 9).

TABLE 9

| | | | | | | | | | | MSDC- | |
|---|---|---|---|---|---|---|---|---|---|---|---|
| | | | | | | | | | | 0602K | |
| | | | | MSDC- | | MSDC- | | MSDC- | | 125 mg | |
| | | | | 0602K | | 0602K | | 0602K | | and | |
| | | Placebo | | 62.5 mg | | 125 mg | | 250 mg | | 250 mg | |
| Disease | Statistic | (N = 94) | | (N = 99) | | (N = 98) | | (N = 101) | | (N = 199) | |
| Primary | | | | | | | | | | | |
| endpoint | | | | | | | | | | | |
| NAS | n/N (%) | 22/74 | (29.7) | 25/84 | (29.8) | 27/82 | (32.9) | 34/86 | (39.5) | 61/168 | (36.3) |
| improvement * | | | | | | | | | | | |
| | Adjusted OR | — | | 0.89 | (0.44, 1.81) | 1.22 | (0.60, 2.48) | 1.64 | (0.83,3.27) | 1.43 | (0.77, 2.64) |
| | (95% CI) † | | | | | | | | | | |
| | p-value | — | | 0.747 | | 0.575 | | 0.158 | | 0.256 | |
| | Adjusted risk | — | | −0.020 | (−0.158, 0.118) | 0.044 | (−0.093, 0.181) | 0.104 | (−0.034, 0.242) | 0.075 | (−0.044, 0.194) |
| | difference | | | | | | | | | | |
| | (95% CI) † | | | | | | | | | | |
| Secondary | | | | | | | | | | | |
| endpoints | | | | | | | | | | | |
| NASH | n/N (%) | 15/74 | (20.3) | 17/84 | (20.2) | 23/82 | (28.0) | 27/86 | (31.4) | 50/168 | (29.8) |
| resolution‡ | | | | | | | | | | | |
| | Adjusted OR | — | | 1.09 | (0.49, 2.42) | 1.50 | (0.70, 3.21) | 1.82 | (0.86, 3.82) | 1.66 | (0.85, 3.24) |
| | (95% CI) † | | | | | | | | | | |
| | p-value | — | | 0.828 | | 0.299 | | 0.116 | | 0.140 | |
| | Adjusted risk | — | | 0.013 | (−0.111, 0.138) | 0.075 | (−0.057, 0.208) | 0.108 | (−0.023, 0.240) | 0.093 | (−0.020, 0.206) |
| | difference | | | | | | | | | | |
| | (95% CI) † | | | | | | | | | | |
| Fibrosis | n/N (%) | 16/74 | (21.6) | 20/84 | (23.8) | 23/82 | (28.0) | 25/86 | (29.1) | 48/168 | (28.6) |
| improvement§ | | | | | | | | | | | |

| Adjusted OR | — | 1.19 | (0.55, 2.55) | 1.45 | (0.69, 3.06) | 1.51 | (0.72, 3.16) | 1.48 | (0.77, 2.86) |
|---|---|---|---|---|---|---|---|---|---|
| (95% CI) † | | | | | | | | | |
| p-value | — | 0.657 | | 0.332 | | 0.270 | | 0.242 | |
| Adjusted risk | — | 0.028 | (−0.102, 0.157) | 0.067 | (−0.066, 0.200) | 0.077 | (−0.056, 0.209) | 0.073 | (−0.042, 0.187) |
| difference | | | | | | | | | |
| (95% CI) † | | | | | | | | | |

| | | | | | | |
|---|---|---|---|---|---|---|
| NAS change | n | 74 | 84 | 83 | 86 | 169 |
| to 12 | | | | | | |

* ≥2-point NAS decrease with a ≥1-point reduction in either ballooning or lobular inflammation and no increase in fibrosis stage.
† Adjusted for diabetes mellitus (yes/no), baseline fibrosis (F1 vs. F2/F3) and baseline NAS (0-8).
‡Hepatocellular ballooning score of 0 and lobular inflammation score of 0-1 with no increase in fibrosis stage.
§≥1-stage decrease in fibrosis with no worsening of lobular inflammation or hepatocellular ballooning.
^{∥}Adjusted for age, sex, diabetes mellitus (yes/no), Vitamin E >= 400 IU (yes/no), baseline fibrosis score (F1 vs. F2/F3) and the corresponding baseline value as appropriate, i.e. baseline NAS (0-8), baseline steatosis score, baseline inflammation score, baseline ballooning score, or baseline fibrosis score (0-4) from repeat read of screening biopsy.
¶Confirmed or not confirmed at baseline and 12 months. Odds ratios for the odds of improvement.

Insulin Sensitivity Effects

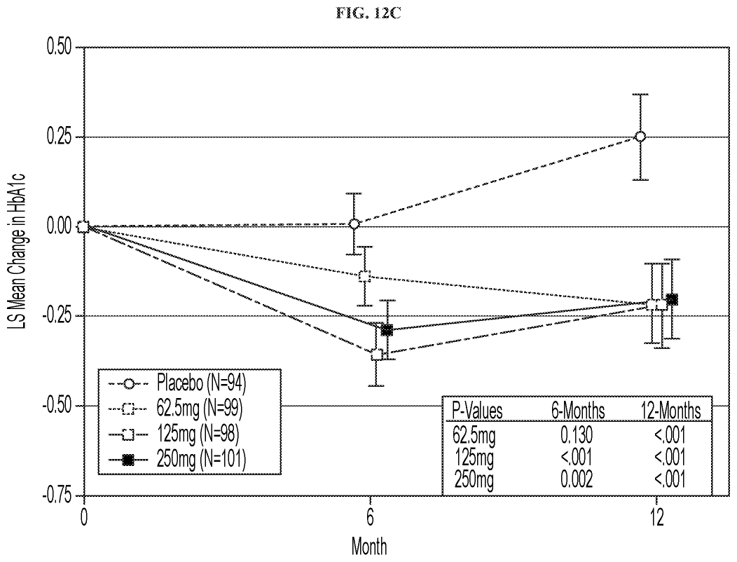

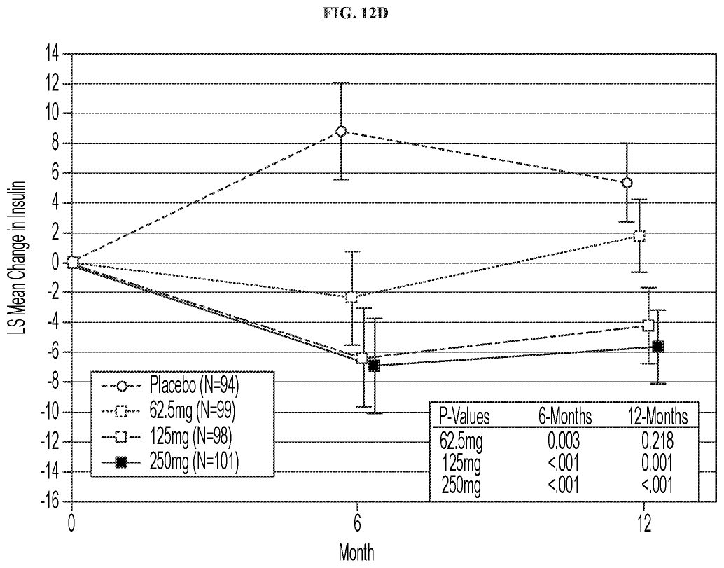

Fasting plasma glucose, HbA1c, fasting plasma insulin, and HOMA-IR, an index of insulin sensitivity derived from insulin and glucose values, all increased (worsened) in patients randomized to placebo (FIG. 12); changes were reduced in a dose-dependent fashion in active treated patients, plateauing at the two highest doses. In patients with poorly-controlled T2D (baseline HbA1c>7%) adjusted mean HbA1c reductions versus placebo at 6 months in the two highest doses were −1.01 (95% CI −1.86-−0.16%) and −0.58% (95% CI −1.35-2.0%), respectively (Table 10).

TABLE 10
Effects of MSDC-0602K on glucose metabolism at 6 months in patients with HbA1C
>7%

| Change in | | | | | |
|---|---|---|---|---|---|
| marker from | | | MSDC-0602K | MSDC-0602K | |
| baseline to 6 | | Placebo | 62.5 mg | 125 mg | MSDC-0602K |
| months | Statistic | (N = 16) | (N = 24) | (N = 17) | 250 mg (N = 23) |
| Glucose, | n | 15 | 21 | 15 | 21 |
| mmol/L | | | | | |
| | LS mean | −1.24 | −2.42 | −3.48 | −3.24 |
| | change* | | | | |
| | LS mean | | −1.18 (−2.77, | −2.23 (−4.00, | −1.99 (−3.59, |
| | difference | | 0.41) | −0.47) | −0.40) |
| | (95% CI)* | | | | |
| | p-value | — | 0.143 | 0.014 | 0.015 |
| Insulin, | n | 15 | 20 | 14 | 19 |
| ulU/mL | | | | | |
| | LS mean | −5.482 | −7.724 | −8.208 | −10.066 |
| | change* | | | | |
| | LS mean | | −2.242 (−21.148, | −2.726 (−23.302, | −4.584 (−23.224, |
| | difference | | 16.664) | 17.850) | 14.056) |
| | (95% CI)* | | | | |
| | p-value | — | 0.813 | 0.792 | 0.624 |
| HOMA IR | n | 15 | 20 | 13 | 19 |
| | LS mean | −4.508 | −6.472 | −8.178 | −8.197 |
| | change* | | | | |
| | LS mean | | −1.964 (−7.344, | −3.669 (−9.729, | −3.689 (−9.024, |
| | difference | | 3.416) | 2.390) | 1.646) |
| | (95% CI)* | | | | |
| | p-value | — | 0.468 | 0.230 | 0.172 |
| HgBA1C,% | n | 15 | 21 | 16 | 22 |
| | LS mean | −0.42 | −0.61 | −1.43 | −1.00 |
| | change* | | | | |
| | LS mean | | −0.18 (−0.97, | −1.01 (−1.86, | −0.58 (−1.35, |
| | difference | | 0.60) | −0.16) | 0.20) |
| | (95% CI)* | | | | |
| | p-value | — | 0.643 | 0.021 | 0.142 |

*Adjusted for age, sex, diabetes mellitus (yes/no), Vitamin E >= 400 IU (yes/no), baseline fibrosis score (F1 vs. F2/F3) and baseline value of marker.

Liver Enzymes

ALT, AST, alkaline phosphatase, and gamma glutamyl transferase (GGT) mean levels changed little in patients randomized to placebo whereas mean levels were persistently reduced in all active dose groups (FIG. 13). While effects on alkaline phosphatase and GGT suggest a graded dose-response, effects of the MSDC-0602K 125 and 250 mg doses on ALT and AST were similar with adjusted mean 6-month reductions relative to placebo of 12.4 (95% CI −19.3-5.5) and 10.5 (95% CI −17.3-−3.7) U/L in ALT, and 7.9 (95% CI −13.8-−1.9) and 5.3 (95% CI −11.1-0.6) U/L in AST, respectively.

The proportion of patients with baseline ALT levels greater than conservative upper normal limits of 19 and 30 U/L for women and men, respectively, whose levels were normal at 12 months was 6.8%, 10.6% (p=0.413), 20.8% p=0.019), and 33.3% (p<0.001) for the placebo, and 62.5, 125, and 250 mg groups, respectively and 27.2% (p=0.0014) for the combined 125 and 250 mg dose groups.

Liver Biomarkers

Mean reductions in fibrosis and apoptosis biomarkers were observed in MSDC-0602K treated patients at 6 and 12 months with mean reductions on these measures in the 125 and 250 mg MSDC-0602K groups combined versus placebo between 0.13 and 0.32 SDs at 6 months and somewhat smaller effects at 12 months (FIG. 14). The average effect of the combined highest doses relative to placebo on ELF, Fib-4, FibroTest, and CK-18 was a reduction of 0.21 (95% CI −0.39-−0.03) SDs at 6 months and 0.17 (95% CI −0.37-0.02) SDs at 12 months.

Weight Gain

MSDC-0602K treated patients gained weight relative to placebo. Unadjusted median weight changes were −0.54, 0.82, 1.10, and 2.28 kg in the placebo and 62.5, 125 and 250 mg MSDC-0602K groups, respectively. Post-hoc regression analyses comparing associations of weight gain with metabolic markers in patients treated with placebo versus MSDC-0602K 125 and 250 mg combined suggest that weight gain was not associated with increases in serum insulin, HbA1C, or AST in patients treated with MSDC-0602K, while in placebo treated patients weight changes were associated with worsening of these measures (FIG. 15).

Patients with Baseline HbA1c>6.5% and AST>27 U/L

Patients with hbA1c>6% and AST>27 U/L were older (58.0 versus 54.8 y), and had higher proportions of patients with T2D (78.2 versus 35.2%), diabetic neuropathy (4.5 versus 0.8%), and hypertension (73.7 versus 62.3%) than those without (Table 11); higher proportions of patients meeting these criteria took RAAS blockers (56.4 versus 40.7%), aspirin (38.5% versus 25.8%), steroids (6.4 versus 1.7%), anti-diabetic medications (70.5% versus 36.0%), and lipid modifiers (57.7% versus 42.8%).

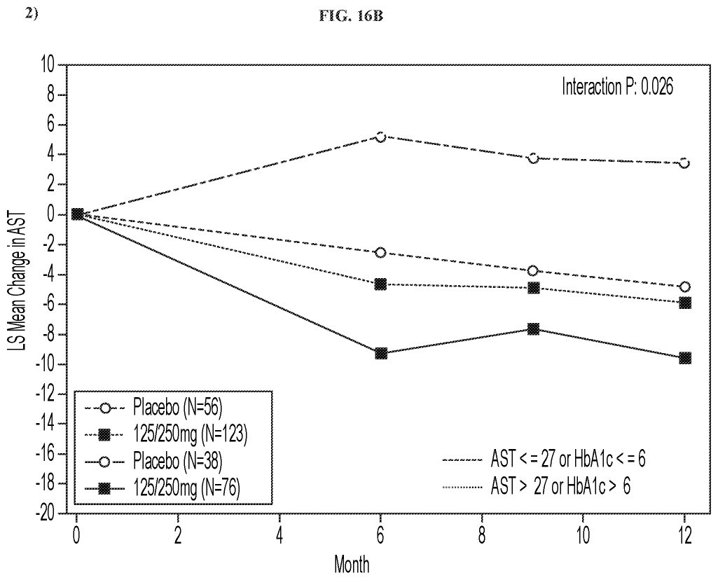

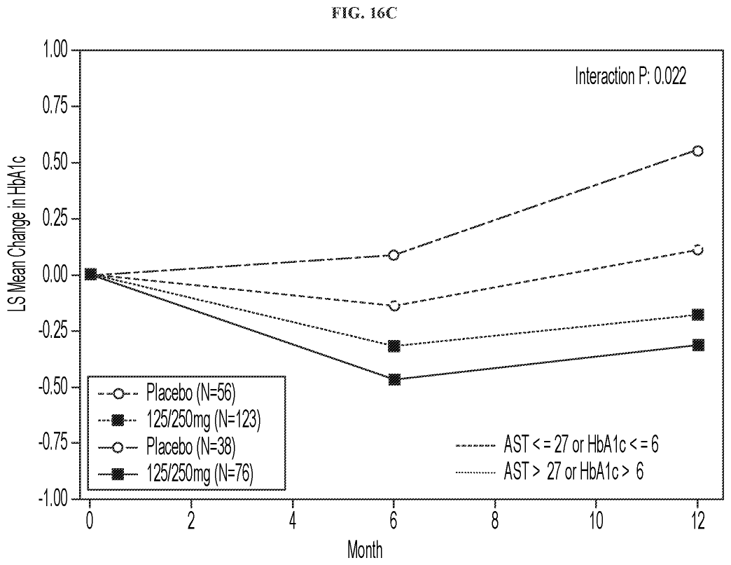

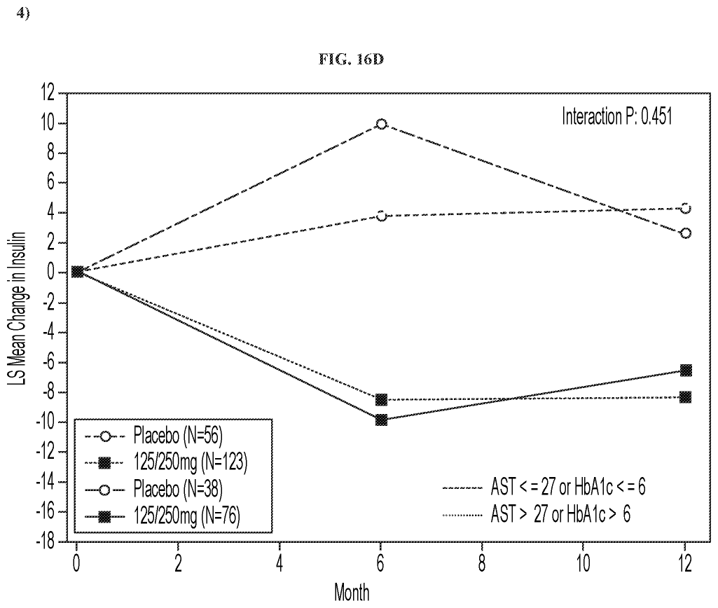

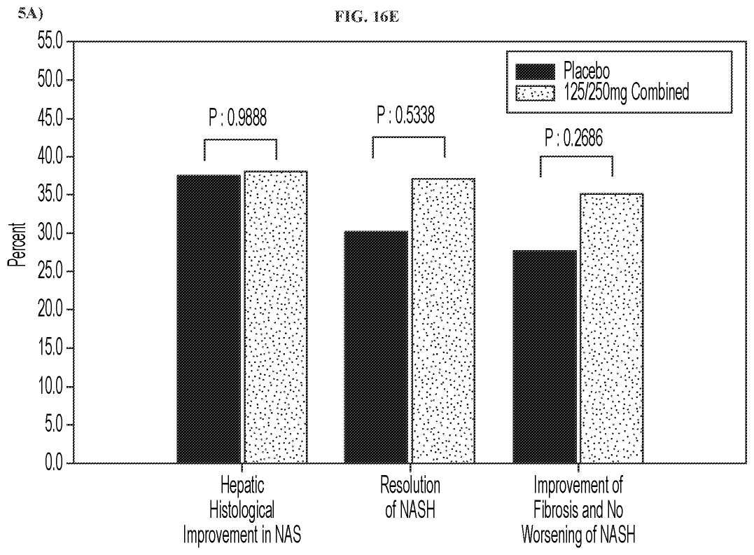

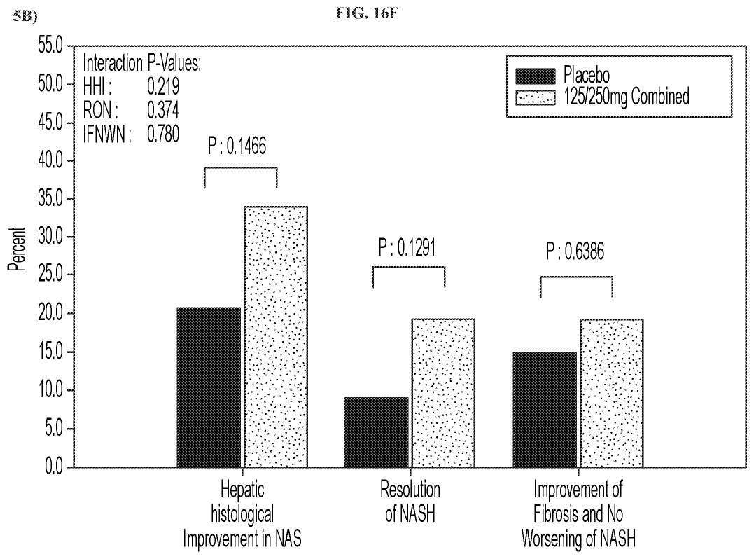

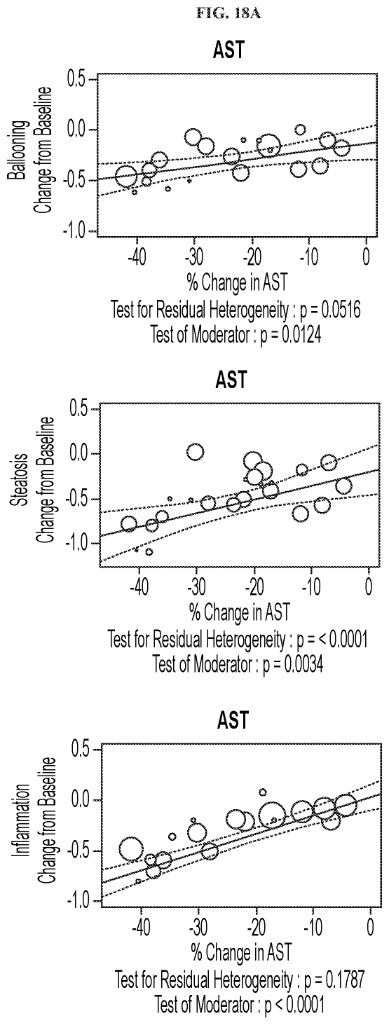

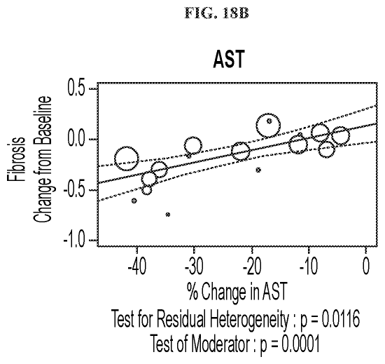

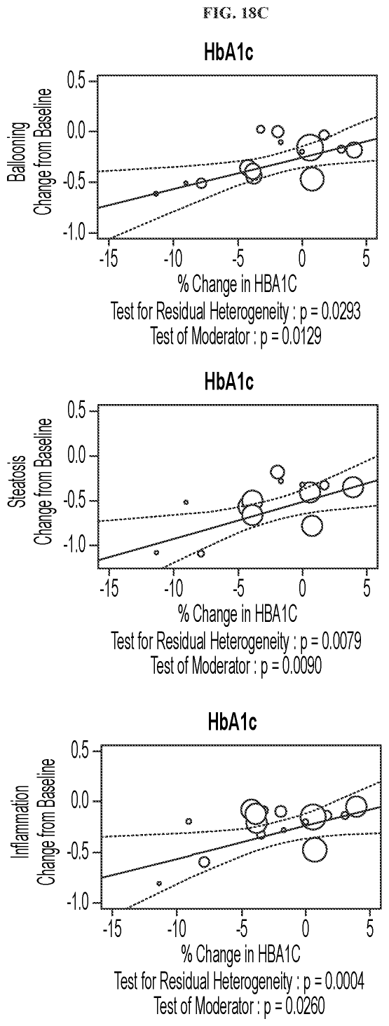

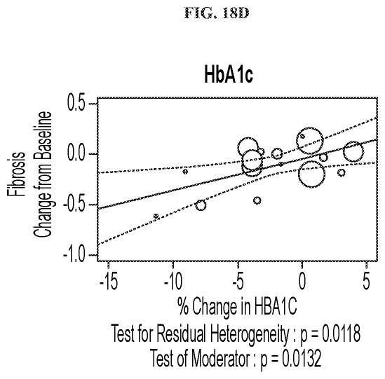

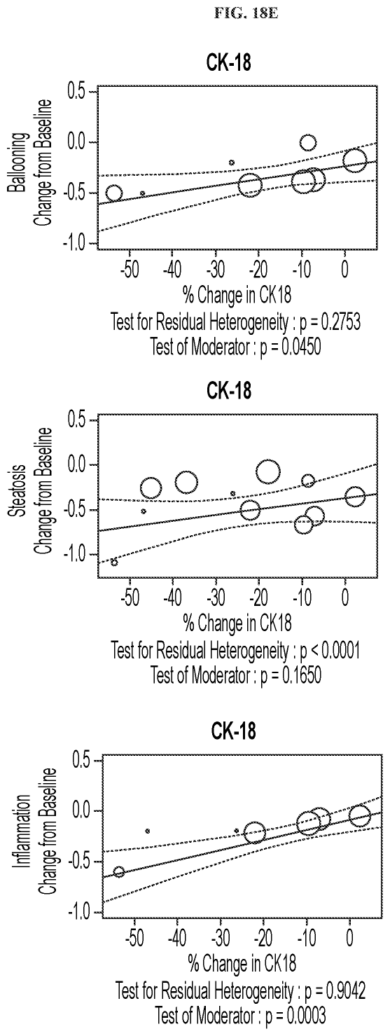

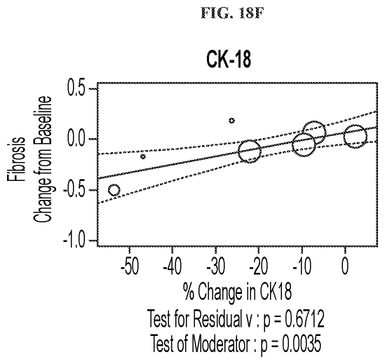

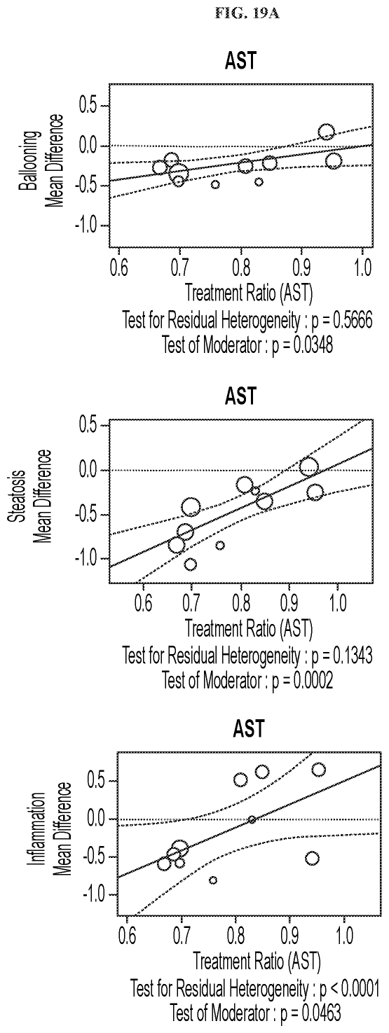

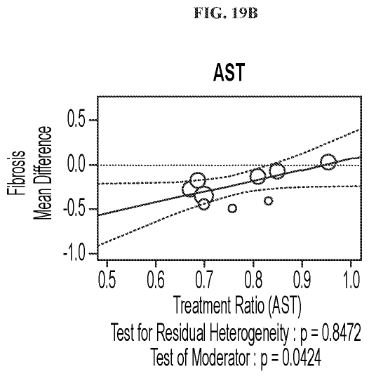

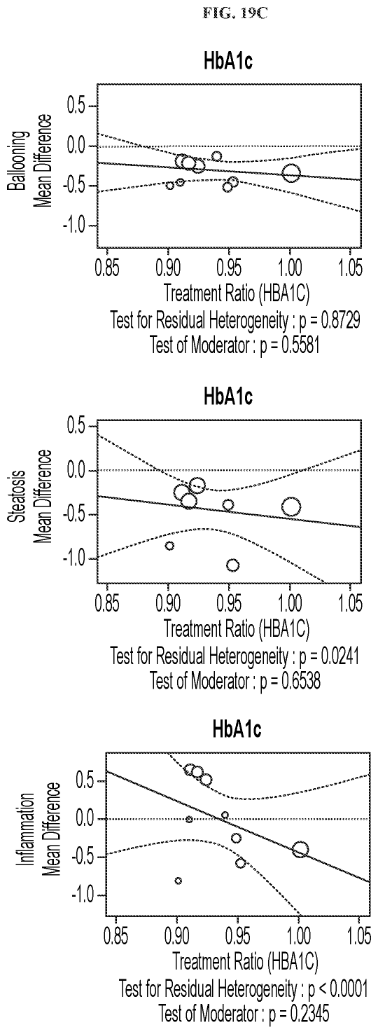

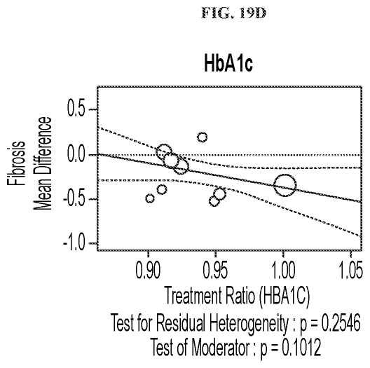

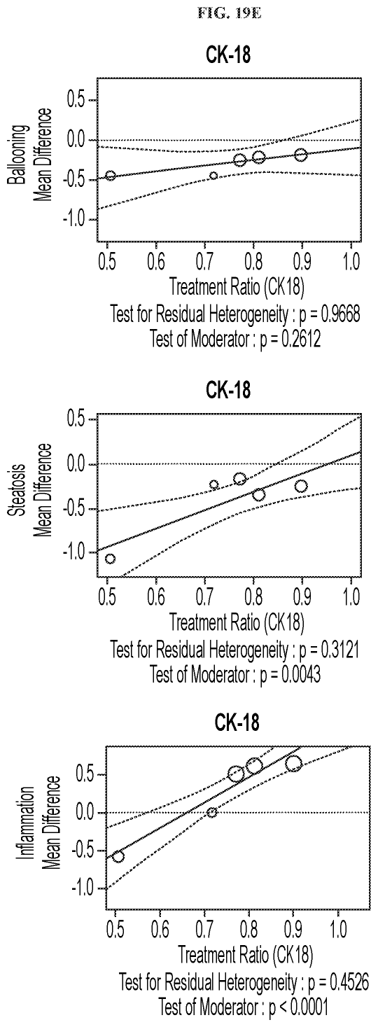

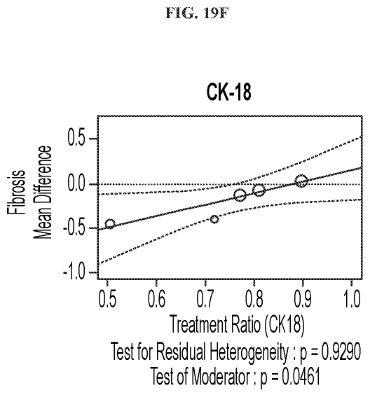

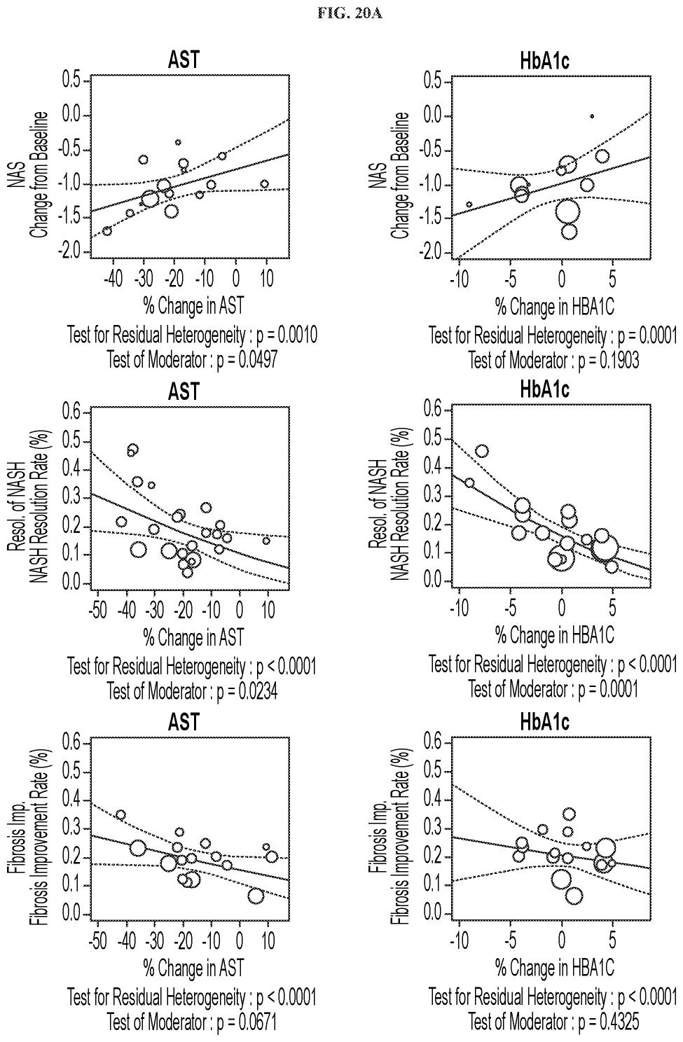

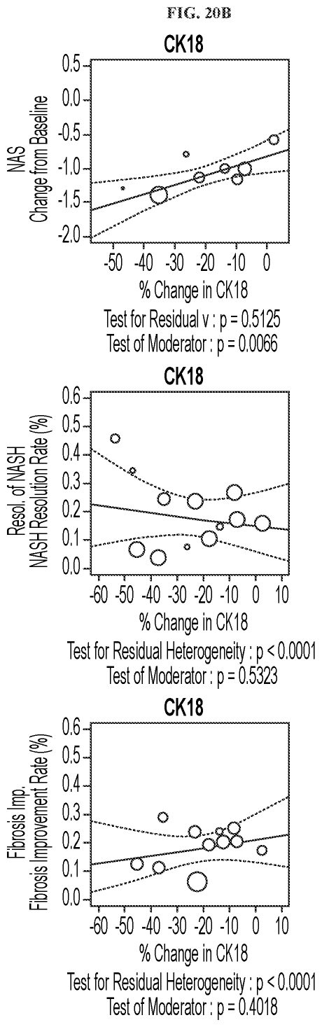

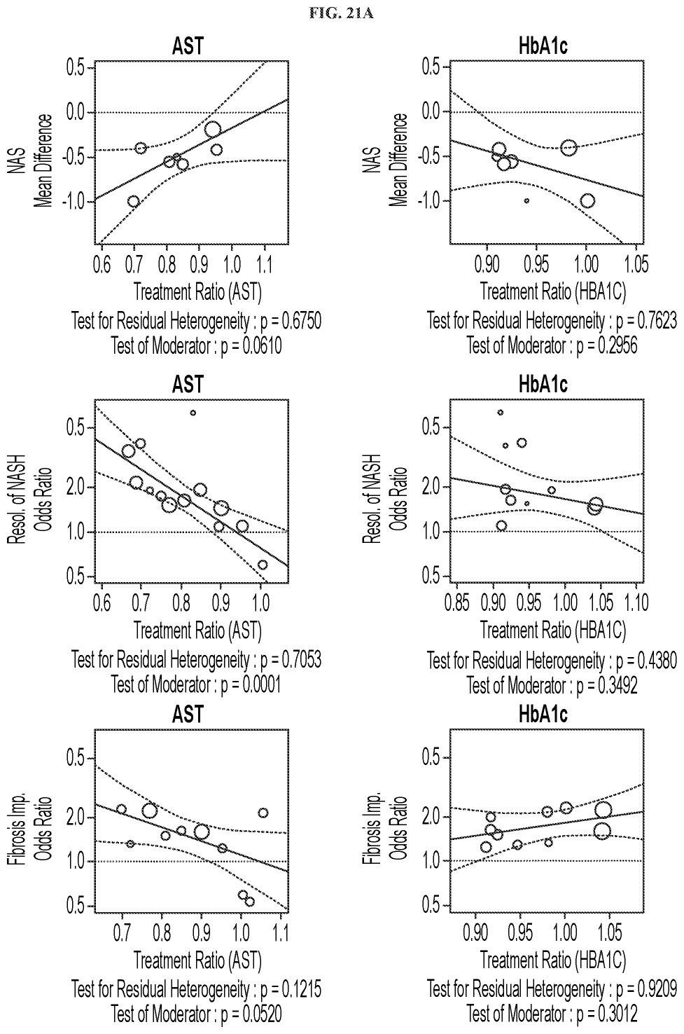

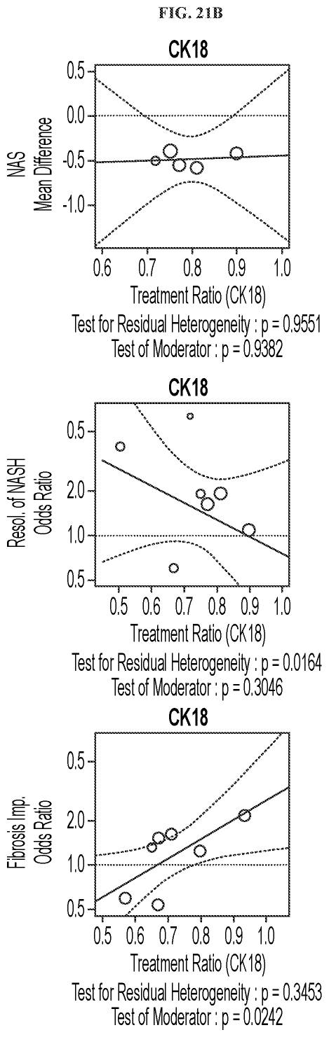

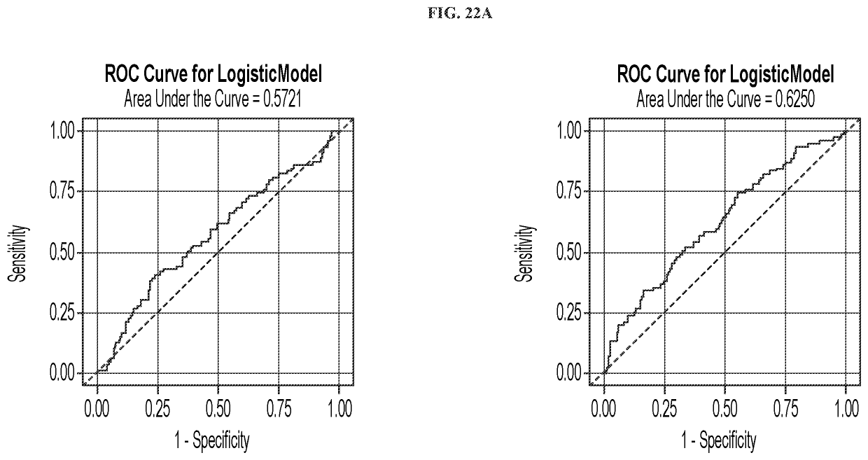

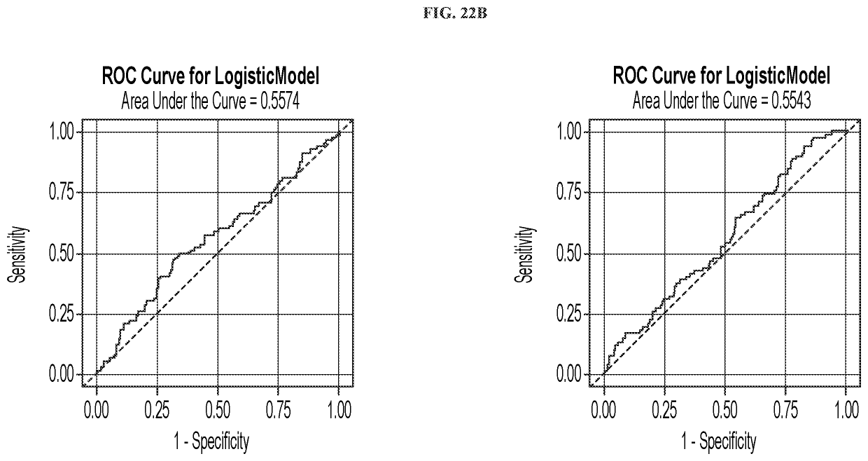

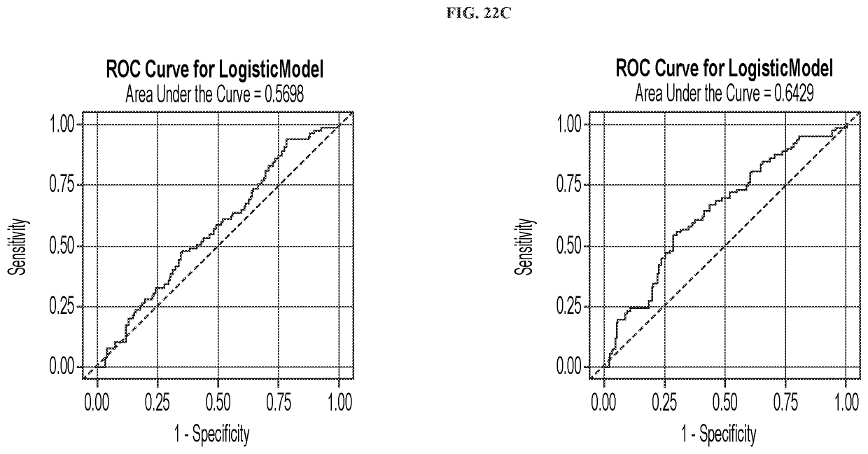

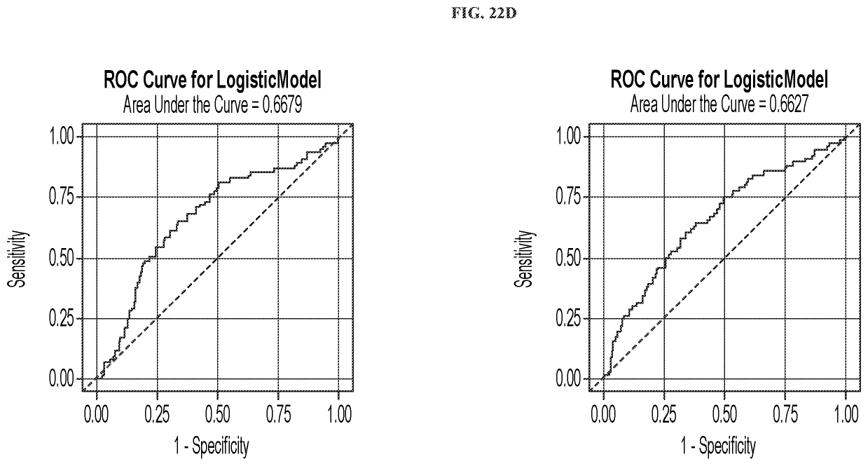

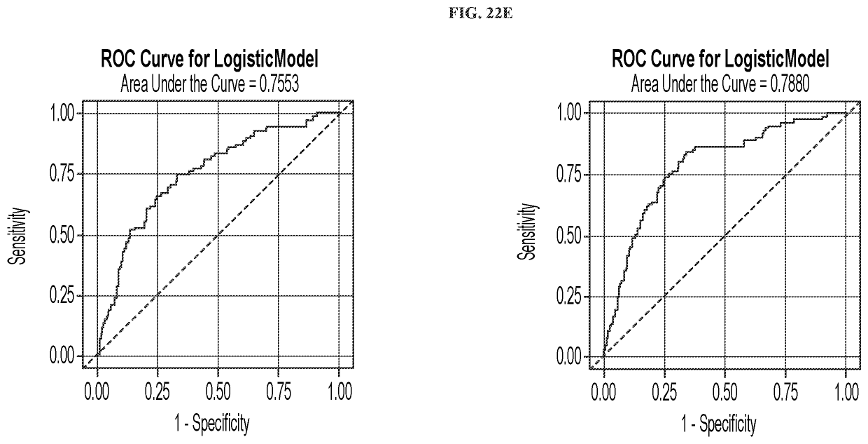

Post-hoc analyses suggest that beneficial effects of MSDC-0602K on liver function and insulin sensitivity, and perhaps liver histology, were greater in patients with baseline AST>27 U/L and HbA1c>6% (FIG. 16), with statistically significantly different effects of 125/250 mg MSDC-0602K in patients meeting and not meeting these criteria on changes in ALT (interaction p=0.026), and HbA1c (interaction p=0.022).

TABLE 11
Baseline characteristics of patients with bassline HgBA1C >6% and AST >27 IU
versus those without

| | | Not AST >27 | AST >27 | |
|---|---|---|---|---|
| | | HbA1c >6 | HbA1c >6 | |
| | Statistic | (N = 236) | (N = 156) | P-Value |
| Age, years | Mean (SD) | 54.8 (11.45) | 58.0 (9.15) | 0.0033 |
| Sex (M:F) | | 102:134 | 62:94 | 0.4946 |
| Race (Black or African American: | | 9:216 | 5:144 | 0.7479 |
| White) | | | | |
| Ethnicity (Hispanic or Latino: | | 64:172 | 55:101 | 0.0863 |
| Not Hispanic or Latino) | | | | |
| Weight, kg | Mean (SD) | 99.9 (20.62) | 99.3 (18.88) | 0.7741 |
| BMI, kg/m2 | Mean (SD) | 35.1 (5.94) | 35.3 (5.50) | 0.7908 |
| Waist Circumference, cm | Mean (SD) | 113.0 (14.20) | 113.9 (12.99) | 0.5173 |
| Medical History | | | | |
| T2DM | n (%) | 35.2 | 78.2 | <.0001 |
| Cardiac disorders | n (%) | 18.6 | 19.9 | 0.7623 |
| Atrial fibrillation | n (%) | 2.5 | 0.6 | 0.1641 |
| Coronary artery disease | n (%) | 3.8 | 6.4 | 0.2413 |
| Hypothyroidism | n (%) | 19.1 | 23.7 | 0.2679 |
| Oedema peripheral | n (%) | 8.5 | 8.3 | 0.9607 |
| Hepatobiliary disorders | n (%) | 80.1 | 74.4 | 0.1818 |
| Cholelithiasis | n (%) | 16.9 | 12.2 | 0.1961 |
| Hepatic steatosis | n (%) | 41.1 | 42.9 | 0.7167 |
| Hepatitis A | n (%) | 1.3 | 3.2 | 0.1850 |
| Hepatitis B | n (%) | 0.4 | 1.9 | 0.1482 |
| Hepatitis C | n (%) | 3.4 | 1.3 | 0.1951 |
| Hyperlipidemia | n (%) | 32.6 | 42.3 | 0.0513 |
| Diabetic neuropathy | n (%) | 0.8 | 4.5 | 0.0185 |
| Depression | n (%) | 27.5 | 26.3 | 0.7833 |
| Chronic kidney disease | n (%) | 0.8 | 1.3 | 0.6752 |
| Asthma | n (%) | 10.6 | 16.0 | 0.1145 |
| Chronic obstructive pulmonary disease | n (%) | 3.8 | 3.2 | 0.7507 |
| Sleep apnoea syndrome | n (%) | 19.1 | 24.4 | 0.2094 |
| Alcohol Use | n (%) | 3.8 | 3.8 | 0.9869 |
| Substance Abuse | n (%) | 1.7 | 1.3 | 0.7445 |
| Hypertension | n (%) | 62.3 | 73.7 | 0.0186 |
| Concomitant Medications | | | | |
| RAAS Blockers | n (%) | 96 (40.7%) | 88 (56.4%) | 0.0023 |
| Aspirin | n (%) | 61 (25.8%) | 60 (38.5%) | 0.0081 |
| Beta Blockers | n (%) | 50 (21.2%) | 35 (22.4%) | 0.7689 |
| Calcium Channel Blockers | n (%) | 35 (14.8%) | 29 (18.6%) | 0.3243 |
| Steroids | n (%) | 4 (1.7%) | 10 (6.4%) | 0.0138 |
| Diuretics | n (%) | 45 (19.1%) | 39 (25.0%) | 0.1612 |
| Drugs for COPD | n (%) | 38 (16.1%) | 35 (22.4%) | 0.1148 |
| Anti-DM | n (%) | 85 (36.0%) | 110 (70.5%) | <.0001 |
| Metformin | n (%) | 62 (26.3%) | 79 (50.6%) | <.0001 |
| Lipid Modifying Agents | n (%) | 101 (42.8%) | 90 (57.7%) | 0.0039 |
| Thyroid Therapy | | 45 (19.1%) | 41 (26.3%) | 0.0911 |

Safety

MSDC-0602K was well tolerated. Treatment discontinuation rates were 17.0% in the placebo group versus 9.1, 12.2 and 12.9% in the 62.5, 125 and 250 mg MSDC-0602K doses, respectively. The frequency of patients suffering one or more adverse events was similar across groups (Table 12). Non-cardiac chest pain, which occurred in 4 total patients (2 placebo, 1 62.5 mg and 1 125 mg), was the only SAE that occurred in >2% of patients in any treatment group.

Adjudicated MACE occurred in one placebo-treated and two 125 mg-treated patients; no adjudicated liver events occurred. Two unrelated deaths were reported, one in placebo and one in the 125 mg group. One patient in the 125 mg group had an adjudicated HF hospitalization. Of note, adverse events of interest based on the profile of TZDs were not observed more commonly in the MSDC-0602K versus placebo treated patients. Adverse events of edema were reported in 7 placebo treated patients versus 6, 11 and 7 patients treated with MSDC-0602K 62.5, 125 and 250 mg, respectively. The frequency of measured pedal edema at each visit was similar to placebo at all time points for all treatment cohorts. Similarly, fractures requiring hospitalization or surgery were observed in 1 placebo-treated patient versus 1, 1 and 2 patients treated with MSDC-0602K 62.5, 125 and 250 mg, respectively. One adjudicated severe hypoglycemia event occurred in the 250 mg group.

TABLE 12
Summary of adverse events in the EMMINENCE study

| | | MSDC- | MSDC- | MSDC- | |
|---|---|---|---|---|---|
| | | 0602K 62.5 | 0602K 125 | 0602K 250 | |
| | Placebo | mg | mg | mg | Total |
| | (N = 94) | (N = 99) | (N = 98) | (N = 101) | (N = 392) |
| | n (%) | n (%) | n (%) | n (%) | n (%) |
| Subjects with any Treatment-Emergent | 75 (79.8) | 78 (78.8) | 76 (77.6) | 77 (76.2) | 306 (78.1) |
| Adverse Event (TEAE) | | | | | |
| Subjects with TEAEs Related to Study Drug [a] | 26 (27.7) | 22 (22.2) | 26 (26.5) | 25 (24.8) | 99 (25.3) |
| Subjects with TEAEs Leading to Study Drug | 4 (4.3) | 2 (2.0) | 3 (3.1) | 1 (1.0) | 10 (2.6) |
| Discontinuation | | | | | |
| Subjects with TEAEs Related to Study Drug | 2(2.1) | 0 (0.0) | 1 (1.0) | 0 (0.0) | 3 (0.8) |
| and Leading to Study Drug Discontinuation [a] | | | | | |
| Treatment-Emergent Serious Adverse Event | | | | | |
| (TESAE) occurring in >2% of subjects in any | | | | | |
| treatment arm. | | | | | |
| MedDRA SOC | | | | | |
| Preferred Term | | | | | |
| General disorders and administration site | 2 (2.1) | 1 ( 1.0) | 2 (2.0) | 1 (1.0) | 6 (1.5) |
| conditions | | | | | |
| Non-cardiac chest pain | 2 (2.1) | 1 ( 1.0) | 1 (1.0) | 0 (0.0) | 4 (1.0) |

[a] Included are (S)AEs considered possibly or probably related to study drug.
Note:
TESAE = Treatment-Emergent Serious Adverse Event;
SOC = System Organ Class;
PT = Preferred Term
Note:
The denominator for percentages is the number of subjects in the Safety Analysis Set for each treatment group.
Note:
Adverse event mapping was based on the MedDRA Version 19.0 thesaurus.

Discussion

In the EMMINENCE study, MSDC-0602K a second generation insulin sensitizer designed to minimize direct binding to PPARγ, but optimized for the ability to modulate the MPC, was administered to patients with liver biopsy confirmed NASH. We tested whether this treatment could be dosed to maximum sensitizing pharmacology without dose-limiting side effects of earlier insulin sensitizers, and whether this pharmacology would benefit the liver as well as improve glycemic control. Results show that the MSDC-0602K tested fully impact metabolic parameters associated with insulin sensitizer pharmacology including reduction of fasting glucose, HbA1c, fasting insulin, and HOMA-IR-without dose-limiting adverse events in this 52-week trial. The two highest doses, 125 mg and 250 mg MSDC-0602K produced similar effects on these parameters.

Current guidance from United States and European regulatory authorities provides for accelerated market approval for a NASH drug based on improvements in liver histology. The EMMINENCE study's primary and secondary outcomes were identified to explore effects on liver histology that might support drug registration. Previous studies showed improvement in histological findings after treatment with TZDs. In EMMINENCE, although results for the primary and secondary endpoints were neutral, some improvements in NAS and steatosis were observed in the 125 and 250 mg MSDC-0602K dose groups. Consistent trends in improvement of liver function/injury (AST, ALT, GGT and alkaline phosphatase) and liver fibrosis markers were also observed with MSDC-0602K treatment. Most of these effects displayed a dose response with 125 and 250 mg doses showing mostly equivalent effects. Taken together, results suggest that MSDC-0602K has a modest beneficial effect on NASH. Importantly, these effects are amplified in patients with higher baseline AST (>27 U/L) and HbA1c (>6%), suggesting that MSDC-0602K's effect is more pronounced in patients with T2D and liver injury. MSDC-0602K had beneficial effects on glucose metabolism markers, demonstrating dose-dependent improvements in glucose, HbA1c, HOMA-IR and insulin levels. Again, patients with higher baseline AST (>27 U/L) and HbA1C (>6%) experienced more pronounced effects of MSDC-0602K. Results were further amplified in patients with baseline HbA1C>7%, a threshold for uncontrolled T2D.

Importantly, in the current study, daily oral administration of MSDC-0602K over 12 months was well tolerated with no increase in adverse event incidence and no excess of side effects associated with PPARγ agonists such as edema and fractures.

Treatment for T2D that is highly effective, administered orally, well-tolerated and without major side effects is still a major unmet need. Other currently available T2D therapies have limited efficacy and significant side effects, such as hypoglycemia, nausea and gastrointestinal side effects, or must be administered subcutaneously. Thus, if confirmed, treatment with MSDC-0602K for glucose control could impact greatly clinical practice.

The ultimate goal of a therapy directed at patients with insulin resistance manifesting as NASH with or without concomitant T2D is improved outcomes. While most compounds in development are focused on preventing adverse liver-related outcomes, including progression to cirrhosis and liver transplant, patients with NASH, and particularly those with NASH and T2D are much more likely to die from cardiovascular disease. The first generation TZD pioglitazone has been shown to reduce adverse cardiovascular events in both diabetic and non-diabetic patients. MSDC-0602K preserves the beneficial effects observed with first generation insulin sensitizers, including improved insulin resistance and decreased insulin levels, which have been shown to be associated with improved cardiovascular outcomes. The lack of PPARγ-related side effects, most notably edema, enables administration of highly efficacious doses without these limitations.

Finally, although MSDC-0602K treatment was associated with weight gain, the weight gain was modest and not associated with the same metabolic consequences associated with weight gain in placebo-treated patients. Support for this seeming paradox was noted in a post-hoc analysis of the PROactive pioglitazone trial which showed that weight gain in subjects was associated with decreased CV risk in the pioglitazone-treated but not control subjects. Rather than an adverse effect, weight gain may be a consequence of insulin sensitization.

Results of this study support conducting an appropriately sized phase 3 study to examine whether this new, apparently well-tolerated, oral therapy can improve CV outcomes in patients with T2D and liver disease.

Conclusions

In the EMMINENCE phase 2b study, administration of MSDC-0602K, a second-generation insulin sensitizer engineered for maximum effect on the MPC while minimizing direct PPARγ agonism was associated with better glucose metabolism, reduced insulin levels and improvement in hepatic injury measures. Safety limitations observed with the first generation insulin sensitizers were not evident, enabling simple oral administration and excellent tolerability.

### Example 7: Formulation of MSDC-0602K. Described Below in TABLE 6 is a Non-Limiting Example of a Tablet Form of MSDC-0602K

TABLE 6

| | Amount per tablet | | | | |
|---|---|---|---|---|---|
| | (mg/tablet) | | | | Quality |
| Component | 62.5 mg | 125 mg | 250 mg | Function | Standard |
| MSDC-0602K, Int. Spec. | 68.900 | 137.800 | 275.600 | Active | GMP |
| | | | | Pharmaceutical | |
| | | | | ingredient | |
| Lactose Anhydrous | 65.525 | 131.050 | 262.100 | Diluent | NF |
| (SuperTab 21 AN) | | | | | |
| Magnesium Stearate | 0.700 | 1.400 | 2.800 | Lubricant | NF |
| Weight of Granules: | 135.125 | 270.250 | 540.500 | | |
| Microcrystalline Cellulose | 29.500 | 59.000 | 118.00 | Diluent | NF |
| (Avicel PH 200) | | | | | |
| Croscarmellose Sodium | 3.375 | 6.750 | 13.500 | Disintegrant | NF |
| (Ac-Di-Sol) | | | | | |
| Providone K-30 | 5.250 | 10.500 | 21.00 | Binder | USP |
| (PVP K-30) | | | | | |
| Colloidal Silicon Dioxide | 0.825 | 1.650 | 3.300 | Glidant | NF |
| (Aerosil 200) | | | | | |
| Magnesium Stearate | 0.925 | 1.850 | 3.700 | Lubricant | NF |
| Core Tablet Weights | 175.000 | 350.000 | 700.000 | | |
| Opadry White (YS-1- | 5.250 | 10.500 | 21.000 | Film Coating | |
| 7003), Int. Spec. | | | | Agent | |
| Opadry Composition⁸ | | | | | |
| Titanium Dioxide | 1.641 | 3.282 | 6.965 | Opacifier | USP |
| Hypromellose 2910 3cP | 1.569 | 3.137 | 6.275 | Film forming | USP |
| | | | | agent | |
| Hypromellose 2910 6cP | 1.569 | 3.137 | 6.275 | Film forming | NF |
| | | | | agent | |
| Polyethiene Glycol 400 | 0.420 | 0.840 | 1.680 | Plasticizer | NF |
| Polysorbate 80 | 0.053 | 0.105 | 0.210 | Wetting agent | NF |
| Purified water¹ | | | | Coating solvent | USP |
| Total Average Coated Tablet | 180.250 | 360.500 | 721.000 | | |
| Weight | | | | | |

¹Water removed during manufacturing

### Example 8. An AST>27 U/L and HbA1c>6% Defines Presumed NASH in Patients with NASH by Imaging

Methods

Data Sources

The NAFLD Prevalence Study was an observational study that enrolled 835 male and female patients aged 18-80 years presenting to the Brook Army Medical Center (BAMC) Gastroenterology clinic for routine colon cancer screening. After providing informed consent, routine clinical chemistries were obtained and patients underwent screening with vibration-controlled transient elastography (VCTE) using FibroScan® (Echosens™, Waltham, Mass., USA) and magnetic resonance imaging (MRI) analyzed with LiverMultiScan™ (Perspectum Diagnostics, South San Francisco, Calif., USA). Two hundred and fifty-seven patients with a Fibroscan liver stiffness measurement ≥7 kPa, magnetic resonance elastography (MRE) liver stiffness measure ≥3 kPa, LiverMultiScan® proton density fat fraction (PDFF)≥5%, or LiverMultiScan® corrected T1 (cT1)≥875 ms (a measure of liver inflammation and fibrosis) were offered a liver biopsy. Liver biopsies were read independently by two readers who formed a consensus on any disagreements using the NASH Clinical Research Network (CRN) scoring system and the SAF (steatosis, activity, and fibrosis) system. NASH diagnosis was defined using the fatty liver inhibition of progression (FLIP) algorithm. The Institutional Review Board of the BAMC approved the study.

EMMINENCE was a randomized, double-blind, placebo-controlled phase 2 trial evaluating the effect of three doses of the insulin sensitizer MSDC-0602K on liver histology in patients with NASH. A total of 1090 patients were screened at 57 United States centers between September 2016 and May 2018. Qualifying liver biopsies, which were obtained for 728 patients, were required within 9 months prior to enrollment. If an historical biopsy was not available (511 patients), a liver biopsy was performed if the patient had aspartate aminotransferase (AST)≥20 U/L, Fibroscan® controlled attenuation parameter (CAP) score ≥270 db/m, and Fibroscan® liver stiffness measure ≥8.5 kPa. All screened patients had routine central laboratory hematology and clinical chemistry measures, while insulin and lipid panel measures were available for only randomized patients. Screening liver biopsies were reviewed by a single, expert hepatopathologist using the NASH CRN scoring system once to qualify the patient for the study and a second time for enrolled patients randomly mixed with follow-up biopsies. The re-read biopsy results were used where available and the qualifying read otherwise. Patients provided written, informed consent and applicable IRBs approved the study which was registered with clinicaltrials.gov as NCT0278444.

Statistical Methods

Patient characteristics are described for patients who underwent a liver biopsy and for whom a NASH diagnosis was either confirmed or not confirmed; continuous variables were compared between groups using t-tests and categorical variables using chi-square tests.

Candidate variables for a multivariable logistic regression model predicting a confirmed NASH diagnosis in each database included clinical characteristics and laboratory data available for the majority of patients biopsied. Some candidate predictors were removed that were collinear or highly correlated with those remaining (including ALT which was removed due to collinearity with the better performing AST). Non-linearity of associations of each continuous predictor with the outcome was assessed by the statistical significance of non-linear components of a restricted cubic spline transformation; where non-linear, associations were modeled using quadratic or cubic polynomial, linear spline, or log 2 transformations chosen based on the Akaike's Information Criterion and plots. Missing values were multiply imputed 10 times assuming multivariate normality, and parameter estimates and associated errors averaged over the 10 datasets using Rubin's rules. Predictors that remained in the majority of the imputation datasets following backwards selection with a 0.05 significance level criterion for staying in the model were selected and the final model run and averaged over the 10 datasets.

As AST and HbA1C were the only two variables to come into both models, a second multivariable model was developed replacing continuous AST and HbA1c measures with a single indicator for having both AST>27 U/L and HbA1c>6%. These cut-offs were derived from the optimal cut-offs on the AUCROC curves (see below).

The area under the receiver-operating characteristic curve (AUROC) is presented for selected parameters. The c-index, which corresponds to the AUROC, is presented for logistic regression models. A value of 1.0 represents perfect discrimination between responders and non-responders, while 0.5 represents no better than chance discrimination. Youden's index, the vertical distance from the noninformative diagonal to the cut-point, was used to select the optimal threshold.

Analyses were conducted using SAS® statistical software version 9.4 (SAS Institute, Cary, N.C., USA). Two-sided p<0.05 was considered statistically significant.

Results

The 257 patients who underwent liver biopsy in the NAFLD Prevalence Study were on average 55.8±6.21 years old, 59.5% were male, and 82.0% were white (Table 1). NASH was diagnosed in 94 (36.6%) patients. The majority of patients (70.0%) were non-Hispanic, with a lower proportion of non-Hispanic patients (58.5% v. 76.7%) among those with a NASH diagnosis. Patients with a confirmed NASH diagnosis were more often obese (88.3% v. 66.9%), and diabetic (37.2% v. 14.7%) than those without NASH. Mean HbA1c (6.37±1.362 v. 5.74±0.730%) and fasting glucose (6.76±2.265 v. 5.75±1.315 mmol/L) were higher in patients with confirmed NASH, as were mean AST (27.6±13.01 v. 22.2±8.79 U/L) and ALT. Mean triglyceride levels were higher in NASH patients (2.18±1.487 v. 1.66±1.140 mmol/L), but LDL, HDL, and total cholesterol were not significantly different.

TABLE 1
Baseline Characteristics of Biopsied Patients in the NAFLD Prevalence Study by NASH Diagnosis

| | | | | NASH Not | | NASH | | | | |
|---|---|---|---|---|---|---|---|---|---|---|
| | N (% | | | Confirmed | | Confirmed | | P- | Total | |
| Parameter | missing) | | Statistic | (N = 163) | | (N = 94) | | value | (N = 257) | |
| Age (years) | 257 | (0.0%) | n | 163 | | 94 | | | 257 | |
| | | | Mean (SD) | 55.5 | (6.34) | 56.3 | (5.99) | 0.3234 | 55.8 | (6.21) |
| | | | Median | 55.0 | (50.0, 60.0) | 56.0 | (51.0, 61.0) | | 55.0 | (50.0, 60.0) |
| | | | (Q1, Q3) | | | | | | | |
| | | | Min, Max | 40, 73 | | 44, 72 | | | 40, 73 | |
| Male sex | 257 | (0.0%) | n (%) | 96 | (58.9%) | 57 | (60.6%) | 0.7840 | 153 | (59.5%) |
| White race | 255 | (0.8%) | n (%) | 128 | (79.5%) | 81 | (86.2%) | 0.1816 | 209 | (82.0%) |
| Non- | 253 | (1.6%) | n (%) | 122 | (76.7%) | 55 | (58.5%) | 0.0023 | 177 | (70.0%) |
| Hispanic | | | | | | | | | | |
| BMI | 257 | (0.0%) | n | 163 | | 94 | | | 257 | |
| (kg · m − 2) | | | | | | | | | | |
| | | | Mean (SD) | 32.48 | (4.966) | 34.22 | (5.114) | 0.0079 | 33.12 | (5.080) |
| | | | Median | 31.92 | (29.33, 35.42) | 33.54 | (31.24, 36.46) | | 32.57 | (29.99, 36.06) |
| | | | (Q1, Q3) | | | | | | | |
| | | | Min, Max | 18.3, 45.5 | | 24.2, 60.4 | | | 18.3, 60.4 | |
| BMI ≥ 30 | 257 | (0.0%) | n (%) | 109 | (66.9%) | 83 | (88.3%) | 0.0001 | 192 | (74.7%) |
| kg · m − 2 | | | | | | | | | | |
| Diabetes | 257 | (0.0%) | n (%) | 24 | (14.7%) | 35 | (37.2%) | <.0001 | 59 | (23.0%) |
| mellitus | | | | | | | | | | |
| Arterial | 257 | (0.0%) | n (%) | 74 | (45.4%) | 46 | (48.9%) | 0.5841 | 120 | (46.7%) |
| Hypertension | | | | | | | | | | |
| Hypercholes- | 257 | (0.0%) | n (%) | 70 | (42.9%) | 47 | (50.0%) | 0.2740 | 117 | (45.5%) |
| terolemia | | | | | | | | | | |
| Elasticity | 255 | (0.8%) | n | 161 | | 94 | | | 255 | |
| (kPa) | | | | | | | | | | |
| | | | Mean (SD) | 6.32 | (2.660) | 7.60 | (3.845) | 0.0020 | 6.79 | (3.202) |
| | | | Median | 5.60 | (4.50, 7.90) | 6.80 | (5.10, 8.80) | | 6.00 | (4.70, 8.10) |
| | | | (Q1, Q3) | | | | | | | |
| | | | Min, Max | 2.3, 16.9 | | 2.8, 25.7 | | | 2.3, 25.7 | |
| CAP | 255 | (0.8%) | n | 161 | | 94 | | | 255 | |
| (db/m) | | | | | | | | | | |
| | | | Mean (SD) | 296.8 | (53.66) | 346.0 | (40.34) | <.0001 | 314.9 | (54.53) |
| | | | Median | 292.0 | (261.0, 336.0) | 352.0 | (315.0, 382.0) | | 317.0 | (276.0, 360.0) |
| | | | (Q1, Q3) | | | | | | | |
| | | | Min, Max | 137, 396 | | 238, 400 | | | 137, 400 | |
| MR | 252 | (1.9%) | n | 160 | | 92 | | | 252 | |
| Elasticity | | | | | | | | | | |
| (kPa) | | | | | | | | | | |
| | | | Mean (SD) | 2.26 | (0.368) | 2.53 | (0.683) | <.0001 | 2.36 | (0.522) |
| | | | Median | 2.20 | (2.00, 2.50) | 2.40 | (2.10, 2.80) | | 2.30 | (2.00, 2.60) |
| | | | (Q1, Q3) | | | | | | | |
| | | | Min, Max | 1.5, 3.3 | | 1.6, 5.7 | | | 1.5, 5.7 | |
| cT1 | 254 | (1.2%) | n | 161 | | 93 | | | 254 | |
| (Version 1) | | | | | | | | | | |
| | | | Mean (SD) | 864.18 | (55.460) | 913.94 | (55.132) | <.0001 | 882.40 | (60.226) |
| | | | Median | 874.45 | (822.13, 901.88) | 907.55 | (878.38, 947.98) | | 886.95 | (841.50. 922.40) |
| | | | (Q1, Q3) | | | | | | | |
| | | | Min, Max | 636.5, 1027.5 | | 786.2, 1050.9 | | | 636.5, 1050.9 | |
| cT1 | 254 | (1.2%) | n | 161 | | 93 | | | 254 | |
| (Version 2) | | | | | | | | | | |
| | | | Mean (SD) | 767.61 | (70.379) | 830.75 | (69.963) | <.0001 | 790.73 | (76.427) |
| | | | Median | 780.64 | (714.24, 815.44) | 822.64 | (785.62, 873.94) | | 796.50 | (738.83, 841.49) |
| | | | (Q1, Q3) | | | | | | | |
| | | | Min, Max | 478.7, 974.9 | | 668.6, 1004.5 | | | 478.7, 1004.5 | |
| LIF | 254 | (1.2%) | n | 161 | | 93 | | | 254 | |
| | | | Mean (SD) | 1.88 | (0.648) | 2.42 | (0.585) | <.0001 | 2.08 | (0.678) |
| | | | Median | 2.00 | (1.30, 2.38) | 2.45 | (2.05, 2.95) | | 2.15 | (1.57, 2.63) |
| | | | (Q1, Q3) | | | | | | | |
| | | | Min, Max | 0.7, 3.3 | | 0.9, 3.3 | | | 0.7, 3.3 | |
| MR | 257 | (0.0%) | n | 163 | | 94 | | | 257 | |
| PDFF (%) | | | | | | | | | | |
| | | | Mean (SD) | 6.56 | (4.704) | 14.87 | (6.413) | <.0001 | 9.60 | (6.711) |
| | | | Median | 5.90 | (2.80, 8.20) | 14.30 | (10.00, 19.00) | | 7.70 | (4.80, 13.80) |
| | | | (Q1, Q3) | | | | | | | |
| | | | Min, Max | 0.8, 23.7 | | 4.8, 33.3 | | | 0.8, 33.3 | |
| MR | 257 | (0.0%) | n (%) | 99 | (60.7%) | 93 | (98.9%) | <.0001 | 192 | (74.7%) |
| PDFF ≥5% | | | | | | | | | | |

| | | | | | | |
|---|---|---|---|---|---|---|
| Aspartate | 248 | (3.5%) | n | 156 | 92 | 248 |
| aminotrans- | | | | | | |
| ferase | | | | | | |
| (U · L − 1) | | | | | | |

| | | | | | | | |
|---|---|---|---|---|---|---|---|
| Mean (SD) | 22.2 | (8.79) | 27.6 | (13.01) | 0.0001 | 24.2 | (10.85) |
| Median | 21.0 | (17.0, 24.5) | 23.0 | (20.0, 32.0) | | 22.0 | (18.0, 27.0) |
| (Q1, Q3) | | | | | | | |

| | | | | | | |
|---|---|---|---|---|---|---|
| | | | Min, Max | 11, 79 | 9, 91 | 9, 91 |
| Alanine | 248 | (3.5%) | n | 156 | 92 | 248 |
| aminotrans- | | | | | | |
| ferase | | | | | | |
| (U · L − 1) | | | | | | |

| | | | | | | | |
|---|---|---|---|---|---|---|---|
| Mean (SD) | 26.5 | (15.10) | 36.5 | (21.59) | <.0001 | 30.2 | (18.40) |
| Median | 23.0 | (17.5, 30.0) | 34.5 | (22.5, 44.5) | | 25.0 | (19.0, 38.0) |
| (Q1, Q3) | | | | | | | |

| | | | | | | |
|---|---|---|---|---|---|---|
| | | | Min, Max | 8, 130 | 10, 180 | 8, 180 |
| Albumin | 248 | (3.5%) | n | 156 | 92 | 248 |
| (g · L − 1) | | | | | | |

| | | | | | | | |
|---|---|---|---|---|---|---|---|
| Mean (SD) | 43.3 | (2.59) | 43.2 | (2.62) | 0.7322 | 43.2 | (2.60) |
| Median | 43.0 | (41.0, 45.0) | 43.0 | (42.0, 45.0) | | 43.0 | (41.0, 45.0) |
| (Q1, Q3) | | | | | | | |

| | | | | | | |
|---|---|---|---|---|---|---|
| | | | Min, Max | 37, 50 | 36, 51 | 36, 51 |
| Platelets | 252 | (1.9%) | n | 160 | 92 | 252 |
| (109 · L − 1) | | | | | | |

| | | | | | | | |
|---|---|---|---|---|---|---|---|
| Mean (SD) | 248.1 | (65.36) | 241.7 | (60.45) | 0.4371 | 245.8 | (63.57) |
| Median | 235.5 | (206.5, 285.5) | 238.5 | (194.0, 283.5) | | 236.5 | (201.5, 285.0) |
| (Q1, Q3) | | | | | | | |

| | | | | | | |
|---|---|---|---|---|---|---|
| | | | Min, Max | 121, 611 | 136, 419 | 121, 611 |
| Cholesterol | 251 | (2.3%) | n | 159 | 92 | 251 |
| (mmol · L − 1) | | | | | | |

| | | | | | | | |
|---|---|---|---|---|---|---|---|
| Mean (SD) | 4.96 | (1.156) | 4.88 | (1.290) | 0.6252 | 4.93 | (1.205) |
| Median | 4.94 | (4.16, 5.64) | 4.90 | (3.84, 5.56) | | 4.91 | (4.01, 5.61) |
| (Q1, Q3) | | | | | | | |

| | | | | | | |
|---|---|---|---|---|---|---|
| | | | Min, Max | 2.1, 9.4 | 2.4, 10.9 | 2.1, 10.9 |
| Cholesterol | 251 | (2.3%) | n | 159 | 92 | 251 |
| in HDL | | | | | | |
| (mmol · L − 1) | | | | | | |

| | | | | | | | |
|---|---|---|---|---|---|---|---|
| Mean (SD) | 1.35 | (0.410) | 1.25 | (0.415) | 0.0845 | 1.31 | (0.413) |
| Median | 1.27 | (1.03, 1.55) | 1.16 | (1.01, 1.46) | | 1.24 | (1.03, 1.53) |
| (Q1, Q3) | | | | | | | |

| | | | | | | |
|---|---|---|---|---|---|---|
| | | | Min, Max | 0.6, 2.8 | 0.6, 3.3 | 0.6, 3.3 |
| Cholesterol | 241 | (6.2%) | n | 154 | 87 | 241 |
| in LDL | | | | | | |
| (mmol · L − 1) | | | | | | |

| | | | | | | | |
|---|---|---|---|---|---|---|---|
| Mean (SD) | 2.83 | (0.923) | 2.64 | (1.037) | 0.1453 | 2.76 | (0.968) |
| Median | 2.70 | (2.15, 3.49) | 2.69 | (1.84, 3.31) | | 2.69 | (2.07, 3.44) |
| (Q1, Q3) | | | | | | | |

| | | | | | | |
|---|---|---|---|---|---|---|
| | | | Min, Max | 0.8, 5.6 | 0.1, 4.8 | 0.1, 5.6 |
| Triglyc- | 251 | (2.3%) | n | 159 | 92 | 251 |
| erides | | | | | | |
| (mmol · L − 1) | | | | | | |

| | | | | | | | |
|---|---|---|---|---|---|---|---|
| Mean (SD) | 1.66 | (1.140) | 2.18 | (1.487) | 0.0022 | 1.85 | (1.299) |
| Median | 1.32 | (0.96, 2.00) | 1.86 | (1.33, 2.42) | | 1.53 | (1.06, 2.18) |
| (Q1, Q3) | | | | | | | |

| | | | | | | |
|---|---|---|---|---|---|---|
| | | | Min, Max | 0.3, 7.4 | 0.8, 12.1 | 0.3, 12.1 |
| Hemoglobin | 252 | (1.9%) | n | 159 | 93 | 252 |
| A1c (%) | | | | | | |

| | | | | | | | |
|---|---|---|---|---|---|---|---|
| Mean (SD) | 5.74 | (0.730) | 6.37 | (1.362) | <.0001 | 5.97 | (1.053) |
| Median | 5.60 | (5.40, 5.90) | 5.90 | (5.50, 6.70) | | 5.70 | (5.40, 6.20) |
| (Q1, Q3) | | | | | | | |

| | | | | | | |
|---|---|---|---|---|---|---|
| | | | Min, Max | 4.6, 9.3 | 4.5, 13.2 | 4.5, 13.2 |
| Insulin | 245 | (4.7%) | n | 154 | 91 | 245 |
| [Units/volume] | | | | | | |
| in Serum or | | | | | | |
| Plasm | | | | | | |
| (uU · mL − 1) | | | | | | |

| | | | | | | | |
|---|---|---|---|---|---|---|---|
| Mean (SD) | 18.60 | (16.318) | 22.64 | (16.068) | 0.0612 | 20.10 | (16.310) |
| Median | 14.60 | (9.90, 20.90) | 17.80 | (12.40, 26.50) | | 15.70 | (11.00, 23.10) |
| (Q1, Q3) | | | | | | | |

| | | | | | | |
|---|---|---|---|---|---|---|
| | | | Min, Max | 2.7, 102.7 | 0.3, 103.4 | 0.3, 103.4 |
| Fasting | 246 | (4.3%) | n | 154 | 92 | 246 |
| glucose | | | | | | |
| (mmol · L − 1) | | | | | | |

| | | | | | | | |
|---|---|---|---|---|---|---|---|
| Mean (SD) | 5.75 | (1.315) | 6.76 | (2.265) | <.0001 | 6.13 | (1.796) |
| Median | 5.44 | (5.05, 5.99) | 5.97 | (5.47, 7.52) | | 5.61 | (5.16, 6.38) |
| (Q1, Q3) | | | | | | | |

| | | | | | | |
|---|---|---|---|---|---|---|
| | | | Min, Max | 4.2, 13.5 | 3.3, 17.0 | 3.3, 17.0 |
| Fib-4 Score | 242 | (5.8%) | n | 152 | 90 | 242 |

| | | | | | | | |
|---|---|---|---|---|---|---|---|
| Mean (SD) | 1.04 | (0.347) | 1.16 | (0.456) | 0.0261 | 1.08 | (0.394) |
| Median | 0.97 | (0.79, 1.29) | 1.07 | (0.88, 1.35) | | 0.99 | (0.82, 1.31) |
| (Q1, Q3) | | | | | | | |

| | | | | | | |
|---|---|---|---|---|---|---|
| | | | Min, Max | 0.4, 2.2 | 0.5, 2.6 | 0.4, 2.6 |
| NAFLD | 242 | (5.8%) | n | 152 | 90 | 242 |
| Fibrosis | | | | | | |
| Score | | | | | | |

| | | | | | | | |
|---|---|---|---|---|---|---|---|
| Mean (SD) | −1.57 | (1.215) | −1.11 | (1.143) | 0.0039 | −1.39 | (1.207) |
| Median | −1.61 | (−2.24, −0.83) | −1.06 | (−1.96, −0.39) | | −1.49 | (−2.11, −0.64) |
| (Q1, Q3) | | | | | | | |

| | | | | | | |
|---|---|---|---|---|---|---|
| | | | Min, Max | −6.2, 1.1 | −3.3, 1.5 | −6.2, 1.5 |
| HOMA IR | 244 | (5.1%) | n | 153 | 91 | 244 |

| | | | | | | | |
|---|---|---|---|---|---|---|---|
| Mean (SD) | 5.10 | (5.867) | 6.83 | (5.359) | 0.0217 | 5.74 | (5.734) |
| Median | 3.64 | (2.33, 5.37) | 5.17 | (3.44, 8.68) | | 4.08 | (2.59, 6.49) |
| (Q1, Q3) | | | | | | | |

| | | | |
|---|---|---|---|
| Min, Max | 0.5, 42.4 | 0.1, 34.4 | 0.1, 42.4 |

P-value from T-test for continuous and from Chi-squared test for discrete variables.

The 728 patients screened with liver biopsy in EMMINENCE were 55.4±11.10 years old on average, 42.6% were male and 87.3% were white (Table 2). The majority of patients (69.5%) were non-Hispanic. NASH was confirmed in 479 (65.8%) patients. Patients with NASH had a higher proportion of white patients (89.7 v. 82.7%) than patients without NASH. More patients with biopsy-confirmed NASH had diabetes (49.0 v. 30.5%). (Weight or BMI was not available for screen failures.) Mean HbA1c (6.39±1.046 v. 5.88±0.831%) and fasting glucose (6.73±2.162 v. 5.94±1.632 mmol/L) were higher in NASH patients (fasting insulin not available in screen failures), as was mean AST (43.9±24.47 v. 30.2±15.35 U/L).

TABLE 2
Baseline Characteristics Biopsy-Screened Patients in EMMINENCE by NASH Diagnosis

| | | | | NASH not | | NASH | | | | |
|---|---|---|---|---|---|---|---|---|---|---|
| | N (% | | | Confirmed | | Confirmed | | P- | Total | |
| Parameter | Missing) | | Statistic | (N = 249) | | (N = 479) | | value | (N = 728) | |
| Age, years | 727 | (0.1%) | n | 249 | | 478 | | . | 727 | |
| | | | Mean (SD) | 54.8 | (11.22) | 55.8 | (11.03) | 0.2671 | 55.4 | (11.10) |
| | | | Median | 56.0 | (48.0, 63.0) | 57.0 | (49.0, 64.0) | . | 56.0 | (49.0, 64.0) |
| | | | (Q1, Q3) | | | | | | | |
| Male Sex | 727 | (0.1%) | n(%) | 115 | (46.2%) | 195 | (40.8%) | 0.1632 | 310 | (42.6%) |
| Hispanic | 725 | (0.4%) | n(%) | 67 | (26.9%) | 154 | (32.4%) | 0.1304 | 221 | (30.5%) |
| White Race | 727 | (0.1%) | n(%) | 206 | (82.7%) | 429 | (89.7%) | 0.0069 | 635 | (87.3%) |
| Diabetes | 727 | (0.1%) | n(%) | 76 | (30.5%) | 234 | (49.0%) | <.0001 | 310 | (42.6%) |
| Chemistry | | | | | | | | . | | |
| AST (U/L) | 725 | (0.4%) | n | 247 | | 478 | | . | 725 | |
| | | | Mean (SD) | 30.2 | (15.35) | 43.9 | (24.47) | <.0001 | 39.2 | (22.74) |
| | | | Median | 26.0 | (21.0, 35.0) | 37.0 | (28.0, 54.0) | . | 33.0 | (24.0, 48.0) |
| | | | (Q1, Q3) | | | | | | | |
| Albumin | 726 | (0.3%) | n | 248 | | 478 | | . | 726 | |
| (g/L) | | | | | | | | | | |
| | | | Mean (SD) | 44.8 | (2.84) | 44.7 | (2.74) | 0.5358 | 44.7 | (2.77) |
| | | | Median | 45.0 | (43.0, 47.0) | 45.0 | (43.0, 47.0) | . | 45.0 | (43.0, 47.0) |
| | | | (Q1, Q3) | | | | | | | |
| Alk. Phos | 726 | (0.3%) | n | 248 | | 478 | | . | 726 | |
| (U/L) | | | | | | | | | | |
| | | | Mean (SD) | 86.2 | (42.69) | 86.3 | (28.75) | 0.9773 | 86.3 | (34.12) |
| | | | Median | 78.0 | (65.0, 95.0) | 82.0 | (67.0, 101.0) | . | 81.0 | (66.0, 99.0) |
| | | | (Q1, Q3) | | | | | | | |
| Creatinine | 725 | (0.4%) | n | 247 | | 478 | | . | 725 | |
| (umol/L) | | | | | | | | | | |
| | | | Mean (SD) | 74.6 | (18.21) | 73.8 | (16.70) | 0.5462 | 74.1 | (17.22) |
| | | | Median | 71.0 | (62.0, 88.0) | 71.0 | (62.0, 80.0) | . | 71.0 | (62.0, 80.0) |
| | | | (Q1, Q3) | | | | | | | |
| HbA1c (%) | 726 | (0.3%) | n | 248 | | 478 | | . | 726 | |
| | | | Mean (SD) | 5.88 | (0.831) | 6.39 | (1.046) | <.0001 | 6.21 | (1.006) |
| | | | Median | 5.70 | (5.40, 6.20) | 6.10 | (5.70, 6.90) | . | 5.90 | (5.50, 6.70) |
| | | | (Q1, Q3) | | | | | | | |
| GGT (U/L) | 726 | (0.3%) | n | 248 | | 478 | | . | 726 | |
| | | | Mean (SD) | 62.0 | (103.17) | 64.8 | (63.79) | 0.6432 | 63.9 | (79.41) |
| | | | Median | 31.0 | (23.0, 60.0) | 43.0 | (31.0, 73.0) | . | 41.0 | (27.0, 67.0) |
| | | | (Q1, Q3) | | | | | | | |
| Glucose | 726 | (0.3%) | n | 248 | | 478 | | . | 726 | |
| (mmol/L) | | | | | | | | | | |
| | | | Mean (SD) | 5.94 | (1.632) | 6.73 | (2.162) | <.0001 | 6.46 | (2.031) |
| | | | Median | 5.45 | (5.10, 6.10) | 6.10 | (5.40, 7.20) | . | 5.80 | (5.30, 6.90) |
| | | | (Q1, Q3) | | | | | | | |
| Potassium | 726 | (0.3%) | n | 248 | | 478 | | . | 726 | |
| (mmol/L) | | | | | | | | | | |
| | | | Mean (SD) | 4.30 | (0.432) | 4.26 | (0.403) | 0.2183 | 4.27 | (0.413) |
| | | | Median | 4.30 | (4.00, 4.60) | 4.20 | (4.00, 4.50) | . | 4.20 | (4.00, 4.50) |
| | | | (Q1, Q3) | | | | | | | |
| Sodium | 726 | (0.3%) | n | 248 | | 478 | | . | 726 | |
| (mmol/L) | | | | | | | | | | |
| | | | Mean (SD) | 139.5 | (2.80) | 139.7 | (2.53) | 0.2718 | 139.7 | (2.63) |
| | | | Median | 139.0 | (138.0, 141.0) | 140.0 | (138.0, 141.0) | . | 140.0 | (138.0, 141.0) |
| | | | (Q1, Q3) | | | | | | | |
| Uric Acid | 726 | (0.3%) | n | 248 | | 478 | | . | 726 | |
| (umol/L) | | | | | | | | | | |
| | | | Mean (SD) | 357.6 | (84.35) | 361.1 | (92.31) | 0.6163 | 359.9 | (89.63) |
| | | | Median | 357.0 | (303.0, 410.0) | 360.0 | (297.0, 422.0) | . | 357.0 | (297.0, 416.0) |
| | | | (Q1, Q3) | | | | | | | |
| Total | 716 | (1.6%) | n | 242 | | 474 | | . | 716 | |
| Bilirubin | | | | | | | | | | |
| (umol/L) | | | | | | | | | | |
| | | | Mean (SD) | 9.73 | (6.845) | 8.85 | (4.625) | 0.0430 | 9.15 | (5.488) |
| | | | Median | 9.00 | (7.00, 12.00) | 9.00 | (5.00, 10.00) | . | 9.00 | (5.00, 10.00) |
| | | | (Q1, Q3) | | | | | | | |
| Total | 726 | (0.3%) | n | 248 | | 478 | | . | 726 | |
| Protein | | | | | | | | | | |
| (g/L) | | | | | | | | | | |
| | | | Mean (SD) | 72.3 | (4.40) | 72.5 | (4.44) | 0.7527 | 72.4 | (4.42) |
| | | | Median | 72.0 | (69.0, 75.0) | 72.0 | (69.0, 75.0) | . | 72.0 | (69.0, 75.0) |
| | | | (Q1, Q3) | | | | | | | |
| Insulin | 393 | (46.0%) | n | 51 | | 342 | | . | 393 | |
| (uIU/mL) | | | | | | | | | | |
| | | | Mean (SD) | 16.2 | (7.11) | 26.4 | (25.59) | 0.0049 | 25.0 | (24.25) |
| | | | Median | 14.0 | (10.8, 20.6) | 20.0 | (14.1, 29.7) | . | 19.1 | (13.3, 27.7) |
| | | | (Q1, Q3) | | | | | | | |
| Lipid Panel | | | | | | | | . | | |
| LDL | 374 | (48.6%) | n | 52 | | 322 | | . | 374 | |
| (mmol/L) | | | | | | | | | | |
| | | | Mean (SD) | 2.81 | (0.951) | 2.66 | (0.974) | 0.3288 | 2.68 | (0.971) |
| | | | Median | 2.75 | (2.16, 3.39) | 2.63 | (1.97, 3.21) | . | 2.66 | (1.99, 3.23) |
| | | | (Q1, Q3) | | | | | | | |
| HDL | 387 | (46.8%) | n | 53 | | 334 | | . | 387 | |
| (mmol/L) | | | | | | | | | | |
| | | | Mean (SD) | 1.21 | (0.306) | 1.19 | (0.331) | 0.7764 | 1.20 | (0.327) |
| | | | Median | 1.16 | (0.98, 1.42) | 1.14 | (0.98, 1.34) | . | 1.14 | (0.98, 1.34) |
| | | | (Q1, Q3) | | | | | | | |
| Triglyc- | 401 | (44.9%) | n | 53 | | 348 | | . | 401 | |
| erides | | | | | | | | | | |
| (mmol/L) | | | | | | | | | | |
| | | | Mean (SD) | 2.10 | (1.675) | 2.12 | (1.572) | 0.9391 | 2.11 | (1.584) |
| | | | Median | 1.87 | (1.17, 2.46) | 1.81 | (1.35, 2.49) | . | 1.81 | (1.32, 2.48) |
| | | | (Q1, Q3) | | | | | | | |
| Hematology | | | | | | | | . | | |
| Hemoglobin | 726 | (0.3%) | n | 248 | | 478 | | . | 726 | |
| (g/L) | | | | | | | | | | |
| | | | Mean (SD) | 141.3 | (15.46) | 140.8 | (13.67) | 0.6483 | 140.9 | (14.30) |
| | | | Median | 141.0 | (132.5, 152.0) | 141.0 | (132.0, 150.0) | . | 141.0 | (132.0, 151.0) |
| | | | (Q1, Q3) | | | | | | | |
| Hematocrit | 725 | (0.4%) | n | 247 | | 478 | | . | 725 | |
| (L/L) | | | | | | | | | | |
| | | | Mean (SD) | 0.42 | (0.041) | 0.42 | (0.037) | 0.7630 | 0.42 | (0.038) |
| | | | Median | 0.42 | (0.39, 0.45) | 0.42 | (0.40, 0.45) | . | 0.42 | (0.39, 0.45) |
| | | | (Q1, Q3) | | | | | | | |
| Platelets | 725 | (0.4%) | n | 248 | | 477 | | . | 725 | |
| (10⁹/L) | | | | | | | | | | |
| | | | Mean (SD) | 250.7 | (66.05) | 247.9 | (73.12) | 0.6167 | 248.8 | (70.75) |
| | | | Median | 249.5 | (205.5, 292.0) | 238.0 | (198.0, 288.0) | . | 241.0 | (200.0, 290.0) |
| | | | (Q1, Q3) | | | | | | | |
| WBC | 726 | (0.3%) | n | 248 | | 478 | | . | 726 | |
| (10⁹/L) | | | | | | | | | | |
| | | | Mean (SD) | 6.82 | (2.037) | 7.15 | (1.968) | 0.0352 | 7.04 | (1.996) |
| | | | Median | 6.41 | (5.49, 7.94) | 6.79 | (5.79, 8.40) | . | 6.72 | (5.70, 8.24) |
| | | | (Q1, Q3) | | | | | | | |
| Lymphocytes | 726 | (0.3%) | n | 248 | | 478 | | . | 726 | |
| (%) | | | | | | | | | | |
| | | | Mean (SD) | 30.2 | (7.68) | 31.0 | (7.88) | 0.1826 | 30.7 | (7.82) |
| | | | Median | 29.4 | (24.9, 36.3) | 30.2 | (25.8, 35.6) | . | 30.0 | (25.4, 35.6) |
| | | | (Q1, Q3) | | | | | | | |
| Neutrophils | 726 | (0.3%) | n | 248 | | 478 | | . | 726 | |
| (%) | | | | | | | | | | |
| | | | Mean (SD) | 60.3 | (8.34) | 59.7 | (8.23) | 0.3884 | 59.9 | (8.27) |
| | | | Median | 60.9 | (55.2, 65.1) | 60.0 | (54.8, 65.7) | . | 60.5 | (54.9, 65.5) |
| | | | (Q1, Q3) | | | | | | | |
| Monocytes | 726 | (0.3%) | n | 248 | | 478 | | . | 726 | |
| (%) | | | | | | | | | | |
| | | | Mean (SD) | 6.2 | (1.82) | 5.9 | (1.57) | 0.0156 | 6.0 | (1.67) |
| | | | Median | 6.1 | (4.9, 7.2) | 5.7 | (4.8, 6.8) | . | 5.8 | (4.8, 6.9) |
| | | | (Q1, Q3) | | | | | | | |
| Eosinophils | 726 | (0.3%) | n | 248 | | 478 | | . | 726 | |
| (%) | | | | | | | | | | |
| | | | Mean (SD) | 2.6 | (1.57) | 2.6 | (1.38) | 0.8882 | 2.6 | (1.45) |
| | | | Median | 2.3 | (1.6, 3.1) | 2.4 | (1.7, 3.5) | . | 2.3 | (1.7, 3.3) |
| | | | (Q1, Q3) | | | | | | | |
| Basophils | 726 | (0.3%) | n | 248 | | 478 | | . | 726 | |
| (%) | | | | | | | | | | |
| | | | Mean (SD) | 0.7 | (0.38) | 0.8 | (0.43) | 0.1057 | 0.8 | (0.42) |
| | | | Median | 0.7 | (0.5, 0.9) | 0.7 | (0.5, 0.9) | . | 0.7 | (0.5, 0.9) |
| | | | (Q1, Q3) | | | | | | | |
| RBC | 726 | (0.3%) | n | 248 | | 478 | | . | 726 | |
| (10¹²/L) | | | | | | | | | | |
| | | | Mean (SD) | 4.7 | (0.43) | 4.7 | (0.42) | 0.8109 | 4.7 | (0.42) |
| | | | Median | 4.7 | (4.4, 5.0) | 4.8 | (4.5, 5.0) | . | 4.8 | (4.4, 5.0) |
| | | | (Q1, Q3) | | | | | | | |
| MCH (pg) | 726 | (0.3%) | n | 248 | | 478 | | . | 726 | |
| | | | Mean (SD) | 29.9 | (2.30) | 29.8 | (1.97) | 0.4391 | 29.8 | (2.09) |
| | | | Median | 30.0 | (29.0, 31.0) | 30.0 | (29.0, 31.0) | . | 30.0 | (29.0, 31.0) |
| | | | (Q1, Q3) | | | | | | | |
| MCHC | 725 | (0.4%) | n | 247 | | 478 | | . | 725 | |
| (g/L) | | | | | | | | | | |
| | | | Mean (SD) | 337.4 | (14.92) | 336.8 | (13.79) | 0.6220 | 337.0 | (14.18) |
| | | | Median | 340.0 | (330.0, 350.0) | 340.0 | (330.0, 350.0) | . | 340.0 | (330.0, 350.0) |
| | | | (Q1, Q3) | | | | | | | |
| MCV (fL) | 725 | (0.4%) | n | 247 | | 478 | | . | 725 | |
| | | | Mean (SD) | 88.7 | (5.30) | 88.4 | (4.53) | 0.4328 | 88.5 | (4.80) |
| | | | Median | 89.0 | (86.0, 92.0) | 89.0 | (86.0, 91.0) | . | 89.0 | (86.0, 92.0) |
| | | | (Q1, Q3) | | | | | | | |
| Biomarkers | | | | | | | | . | | |
| APRI | 394 | (45.9%) | n | 51 | | 343 | | . | 394 | |
| | | | Mean (SD) | 0.46 | (0.233) | 0.57 | (0.342) | 0.0250 | 0.56 | (0.332) |
| | | | Median | 0.41 | (0.31, 0.55) | 0.48 | (0.35, 0.70) | . | 0.48 | (0.34, 0.69) |
| | | | (Q1, Q3) | | | | | | | |
| CRP (mg/L) | 401 | (44.9%) | n | 53 | | 348 | | . | 401 | |
| | | | Mean (SD) | 5.33 | (6.002) | 7.14 | (9.648) | 0.1856 | 6.90 | (9.264) |
| | | | Median | 3.57 | (1.27, 6.33) | 4.21 | (1.85, 8.64) | . | 4.12 | (1.83, 7.83) |
| | | | (Q1, Q3) | | | | | | | |
| CK-18 | 400 | (45.1%) | n | 53 | | 347 | | . | 400 | |
| [M65] | | | | | | | | | | |
| (U/L) | | | | | | | | | | |
| | | | Mean (SD) | 756.2 | (483.68) | 974.3 | (541.55) | 0.0059 | 945.4 | (538.79) |
| | | | Median | 647.6 | (381.3, 986.4) | 864.5 | (572.0, 1214.7) | . | 841.7 | (551.9, 1196.5) |
| | | | (Q1, Q3) | | | | | | | |
| ELF | 397 | (45.5%) | n | 52 | | 345 | | . | 397 | |
| | | | Mean (SD) | 9.49 | (1.055) | 9.77 | (0.939) | 0.0488 | 9.74 | (0.958) |
| | | | Median | 9.27 | (8.63, 10.27) | 9.66 | (9.03, 10.37) | . | 9.63 | (8.99, 10.37) |
| | | | (Q1, Q3) | | | | | | | |
| Fib-4 | 394 | (45.9%) | n | 51 | | 343 | | . | 394 | |
| | | | Mean (SD) | 1.35 | (0.801) | 1.52 | (0.758) | 0.1375 | 1.50 | (0.765) |
| | | | Median | 1.12 | (0.84, 1.64) | 1.41 | (0.98, 1.86) | . | 1.37 | (0.95, 1.83) |
| | | | (Q1, Q3) | | | | | | | |
| FibroTest | 394 | (45.9%) | n | 51 | | 343 | | . | 394 | |
| Score | | | | | | | | | | |
| | | | Mean (SD) | 0.30 | (0.217) | 0.32 | (0.203) | 0.5594 | 0.32 | (0.205) |
| | | | Median | 0.22 | (0.11, 0.48) | 0.26 | (0.16, 0.44) | . | 0.26 | (0.16, 0.45) |
| | | | (Q1, Q3) | | | | | | | |
| HOMA-IR | 387 | (46.8%) | n | 49 | | 338 | | . | 387 | |
| | | | Mean (SD) | 4.29 | (2.317) | 8.55 | (11.464) | 0.0101 | 8.01 | (10.835) |
| | | | Median | 3.49 | (2.63, 5.41) | 5.73 | (3.71, 9.32) | . | 5.40 | (3.60, 8.88) |
| | | | (Q1, Q3) | | | | | | | |
| AST > | 725 | (0.4%) | n (%) | 28 | (11.3%) | 191 | (40.0%) | <.0001 | 219 | (30.2%) |
| 27 U/L, | | | | | | | | | | |
| HbA1c > 6% | | | | | | | | | | |

^{#} P-value from T-test for continuous variables or from χ²-test for categorical variables.

Among the candidate predictors in the NAFLD Prevalence Study, BMI, AST, triglycerides, HbA1c, and fasting glucose were found to have statistically significant associations with biopsy-confirmed NASH without adjustment for other variables (Table 3a). Triglycerides were found to have a non-linear association with the log odds of NASH diagnosis, with a steep increase in risk (odds ratio (OR) of 11.24 (95% CI 3.39-37.24) per 1-mmol/L increment in triglyceride level) up to a value of 1.5 mmol/L and then a leveling-off at higher values (OR 1.40, 95% CI 0.82-1.32). After backwards selection, the final multivariable model included AST (OR 1.25, 95% CI 1.07-1.46 per 5 U/L increment), HbA1c (OR 1.81, 95% CI 1.30-2.51 per 1% increment), and triglycerides with ORs per 1 mmol/L increment of 12.54 (95% CI 3.47-45.29) below 1.5 mmol/L and 0.85 (95% CI 0.64-1.14) above 1.5 mmol/L. The multivariable model's (Model 1) c-index was 0.7525.

TABLE 3a
Multivariable Model for NASH Diagnosis in the NAFLD Prevalence
Study-Model 1

| | Odds ratio | Univariable Model | | Multivariable Model 1 | |
|---|---|---|---|---|---|
| Parameter | for a change of | Odds ratio (95% CI) | P-value | Odds ratio (95% CI) | P-value |
| Age (years) | 5 | 1.11 (0.90, 1.36) | 0.3233 | | |
| Male sex | Yes vs. No | 1.08 (0.64, 1.81) | 0.7842 | | |
| White race | Yes vs. No | 1.62 (0.80, 3.26) | 0.1812 | | |
| BMI (kg · m-2) | 2 | 1.15 (1.03, 1.27) | 0.0104 | | |
| Arterial Hypertension | Yes vs. No | 1.15 (0.69, 1.92) | 0.5846 | | |
| Hypercholesterolemia | Yes vs. No | 1.33 (0.80, 2.21) | 0.2755 | | |
| Aspartate aminotransferase | 5 | 1.29 (1.12, 1.49) | 0.0005 | 1.25 (1.07, 1.46) | 0.0047 |
| (U · L-1) | | | | | |
| Albumin (g · L-1) | 2 | 0.98 (0.80, 1.19) | 0.8310 | | |
| Cholesterol in HDL | 1 | 0.56 (0.29, 1.09) | 0.0890 | | |
| (mmol · L-1) | | | | | |
| Cholesterol in LDL | 1 | 0.83 (0.64, 1.08) | 0.1694 | | |
| (mmol · L-1) | | | | | |
| Triglycerides ≤1.5 | 1 | 11.24 (3.39, 37.24) | <.0001 | 12.54 (3.47, 45.29) | 0.0006 |
| mmol · L-1^{†} | | | | | |
| Triglycerides >1.5 | 1 | 1.04 (0.82, 1.32) | | 0.85 (0.64, 1.14) | |
| mmol · L-1^{†} | | | | | |
| Hemoglobin A1c (%) | 1 | 1.90 (1.39, 2.60) | <.0001 | 1.81 (1.30, 2.51) | 0.0005 |
| Fasting glucose (mmol · L-1) | 1 | 1.41 (1.18, 1.69) | 0.0003 | | |
| Insulin [Units/volume] in | 5 | 1.07 (0.99, 1.16) | 0.0886 | | |
| Serum or Plasm (uU · mL-1) | | | | | |
| C-index | | | | 0.7525 | |

Results from logistic regression models combined across 10 imputed data sets.
^{†}Non-linear association modeled as linear spline.

In the NAFLD Prevalence Study, AUROCs for AST and HbA1c taken individually were 0.654 and 0.671, respectively, with optimal cut-points of 25 U/L for AST and 5.9% for HbA1c. Unadjusted for other variables, having both AST>27 U/L and HbA1c>6%, was significantly associated (OR 4.21, 95% CI 1.70-10.44) with a NASH diagnosis (Table 3b). This indicator, with adjusted OR 2.98 (95% CI 1.07-8.25), was selected in a second multivariable model along with triglycerides and fasting glucose. The c-index for this multivariable model (Model 2) was 0.7280. The performance characteristics for the AST>27 U/L and HbA1C>6% criterion for the diagnosis of NASH were AUC: 0.565 (95% CI 0.522-0.608), sensitivity: 0.176, specificity: 0.954, positive predictive value (PPV): 0.696 and negative predictive value (NPV): 0.659.

TABLE 3b
Multivariable Model for NASH Diagnosis in the NAFLD Prevalence Study-Model
2

| | Odds ratio | Univariable Model | | Multivariable Model 2 | |
|---|---|---|---|---|---|
| Parameter | for a change of | Odds ratio (95% CI) | P-value | Odds ratio (95% CI) | P-value |
| Age (years) | 5 | 1.11 (0.90, 1.36) | 0.3233 | | |
| Male sex | Yes vs. No | 1.08 (0.64, 1.81) | 0.7842 | | |
| White race | Yes vs. No | 1.62 (0.80, 3.26) | 0.1812 | | |
| BMI (kg · m-2) | 2 | 1.15 (1.03, 1.27) | 0.0104 | | |
| Arterial Hypertension | Yes vs. No | 1.15 (0.69, 1.92) | 0.5846 | | |
| Hypercholesterolemia | Yes vs. No | 1.33 (0.80, 2.21) | 0.2755 | | |
| AST >27 U · L-1 and HbA1c | Yes vs. No | 4.21 (1.70, 10.44) | 0.0022 | 2.98 (1.07, 8.25) | 0.0371 |
| >6 % | | | | | |
| Albumin (g · L-1) | 2 | 0.98 (0.80, 1.19) | 0.8310 | | |
| Cholesterol in HDL | 1 | 0.56 (0.29, 1.09) | 0.0890 | | |
| (mmol · L-1) | | | | | |
| Cholesterol in LDL | 1 | 0.83 (0.64, 1.08) | 0.1694 | | |
| (mmol · L-1) | | | | | |
| Triglycerides ≤1.5 mmol · L-1^{†} | 1 | 11.24 (3.39, 37.24) | <.0001 | 13.55 (3.71, 49.54) | 0.0004 |
| Triglycerides >1.5 mmol · L-1^{†} | 1 | 1.04 (0.82, 1.32) | | 0.86 (0.65, 1.16) | |
| Fasting glucose (mmol · L-1) | 1 | 1.41 (1.18, 1.69) | 0.0003 | 1.29 (1.07, 1.56) | 0.0081 |
| Insulin [Units/volume] in | 5 | 1.07 (0.99, 1.16) | 0.0886 | | |
| Serum or Plasm (uU · mL-1) | | | | | |
| C-index | | | | 0.7280 | |

Results from logistic regression models combined across 10 imputed data sets.
^{†}Non-linear association modeled as linear spline.

Among the candidate predictors in the EMMINENCE study, white race, AST, fasting glucose, HbA1c, WBC, and GGT were found to have statistically significant unadjusted associations with NASH diagnosis (Table 4a). GGT had a non-linear association with the log odds of NASH, with an increase in risk for values up to 100 U/L (OR 1.18, 95% CI 1.10-1.26 per 10 U/L increment) that leveled off at higher values (OR 0.95, 95% CI 0.92-0.99). The final multivariable model included white race (OR 1.81, 95% CI 1.10-2.98), AST (OR 1.27, 95% CI 1.19-1.36 per 5 U/L increment), HbA1c (OR 1.93, 95% CI 1.56-2.39 per 1% increment), WBC (OR 1.11, 95% CI 1.01-1.22 per 109/L increment), and non-linear associations with sodium (umbrella) and GGT. Multivariable Model 1's c-index was 0.7655.

TABLE 4a
Multivariable Model for NASH Diagnosis in EMMINENCE-Model 1

| | | Univariable model | | Multivariable model | |
|---|---|---|---|---|---|
| | Odds ratio | Odds ratio | | Odds ratio | |
| Parameter | for a change of | (95% CI) | P-value | (95% CI) | P-value |
| Age | 5 | 1.04 (0.97, 1.11) | 0.2572 | | |
| Male Sex | Yes vs. No | 0.80 (0.59, 1.09) | 0.1639 | | |
| Hispanic | Yes vs. No | 1.30 (0.93, 1.82) | 0.1314 | | |
| White race | Yes vs. No | 1.83 (1.17, 2.84) | 0.0077 | 1.81 (1.10, 2.98) | 0.0189 |
| AST (U/L) | 5 | 1.25 (1.17, 1.32) | <.0001 | 1.27 (1.19, 1.36) | <.0001 |
| Albumin (g/L) | 2 | 0.97 (0.87, 1.08) | 0.5538 | | |
| Glucose (mmol/L) | 1 | 1.30 (1.16, 1.45) | <.0001 | | |
| Alk. Phos (U/L) | 10 | 1.02 (0.96, 1.07) | 0.5776 | | |
| Platelets (10⁹/L) ^{#} | 241.00 vs. 200.00 | 0.88 (0.76, 1.01) | 0.2602 | | |
| | 291.00 vs. 241.00 | 0.90 (0.74, 1.08) | | | |
| Hematocrit (L/L) ^{‡} | 0.42 vs. 0.39 | 1.03 (0.90, 1.18) | 0.2380 | | |
| | 0.45 vs. 0.42 | 0.92 (0.80, 1.06) | | | |
| HbA1c (%) | 1 | 1.87 (1.53, 2.28) | <.0001 | 1.93 (1.56, 2.39) | <.0001 |
| Sodium (mmol/L)^{‡} | 140.00 vs. 138.00 | 1.11 (0.98, 1.25) | 0.1320 | 1.17 (1.01, 1.34) | 0.0531 |
| | 141.00 vs. 140.00 | 1.02 (0.96, 1.08) | | 1.03 (0.97, 1.11) | |
| Hemoglobin (g/L) | 10 | 0.97 (0.88, 1.09) | 0.6412 | | |
| Creatinine (umol/L) | 5 | 0.99 (0.94, 1.03) | 0.5725 | | |
| Uric Acid (umol/L) | 20 | 1.01 (0.97, 1.04) | 0.6206 | | |
| RBC (10¹²/L) | 1 | 1.04 (0.72, 1.50) | 0.8219 | | |
| Total Protein (g/L) | 5 | 1.03 (0.86, 1.22) | 0.7648 | | |
| Lymphocytes (%) | 2 | 1.03 (0.99, 1.07) | 0.1806 | | |
| Potassium (mmol/L) | 1 | 0.80 (0.55, 1.15) | 0.2294 | | |
| Total Bilirubin (umol/L) | 1 | 0.98 (0.95, 1.01) | 0.2908 | | |
| WBC (10⁹L) | 1 | 1.11 (1.02, 1.20) | 0.0166 | 1.11 (1.01, 1.22) | 0.0326 |
| GGT ≤100 U/L ^{†} | 10 | 1.18 (1.10, 1.26) | <.0001 | 1.02 (0.94, 1.10) | 0.0118 |
| GGT >100 U/L^{†} | 10 | 0.95 (0.92, 0.99) | | 0.94 (0.91, 0.98) | |
| C-index | | | | 0.7655 | |

Results from logistic regression models combined across 10 imputed data sets.
^{†} Non-linear association modeled as linear spline.
^{‡} Non-linear association modeled as quadratic polynomial. Effect sizes for 75th percentile vs. median and for median vs. 25th percentile are presented.
^{#} Non-linear association modeled as cubic polynomial. Effect sizes for 75th percentile vs. median and for median vs. 25th percentile are presented.

In patients screened with biopsy for EMMINENCE, AUROCs for AST and HbA1c taken individually were 0.711 and 0.660, respectively, with optimal cut-points of 29 U/L for AST and 5.6% for HbA1c. Unadjusted for other variables, having both AST>27 U/L and HbA1c>6% was significantly associated (OR 5.20, 95% CI 3.37-8.03) with a NASH diagnosis (Table 4b). In a second multivariable model (Model 2) that included white race, fasting glucose, lymphocytes, potassium, WBC, and GGT, this indicator was statistically significantly associated with NASH (OR 3.84, 95% CI 2.40-6.15). The c-index for multivariable Model 2 was 0.7209. The performance characteristics for the AST>27 U/L and HbA1C>6% criterion for the diagnosis of NASH were AUC: 0.643 (95% CI 0.614-0.673), sensitivity: 0.400, specificity: 0.887, PPV: 0.872 and NPV: 0.432.

TABLE 4b
Multivariable Model for NASH Diagnosis in EMMINENCE-Model 2

| | | Univariable model | | Multivariable model | |
|---|---|---|---|---|---|
| | Odds ratio | Odds ratio | | Odds ratio | |
| Parameter | for a change of | (95% CI) | P-value | (95% CI) | P-value |
| Age | 5 | 1.04 (0.97, 1.11) | 0.2572 | | |
| Male Sex | Yes vs. No | 0.80 (0.59, 1.09) | 0.1639 | | |
| Hispanic | Yes vs. No | 1.30 (0.93, 1.82) | 0.1314 | | |
| White race | Yes vs. No | 1.83 (1.17, 2.84) | 0.0077 | 1.89 (1.16, 3.06) | 0.0105 |
| Albumin (g/L) | 2 | 0.97 (0.87, 1.08) | 0.5538 | | |
| Glucose (mmol/L) | 1 | 1.30 (1.16, 1.45) | <.0001 | 1.17 (1.04, 1.30) | 0.0064 |
| Alk. Phos (U/L) | 10 | 1.02 (0.96, 1.07) | 0.5776 | | |
| Platelets (10⁹/L) ^{#} | 241.00 vs. 200.00 | 0.88 (0.76, 1.01) | 0.2602 | | |
| | 291.00 vs. 241.00 | 0.90 (0.74, 1.08) | | | |
| Hematocrit (L/L) ^{‡} | 0.42 vs. 0.39 | 1.03 (0.90, 1.18) | 0.2380 | | |
| | 0.45 vs. 0.42 | 0.92 (0.80, 1.06) | | | |
| Sodium (mmol/L)^{‡} | 140.00 vs. 138.00 | 1.11 (0.98, 1.25) | 0.1320 | | |
| | 141.00 vs. 140.00 | 1.02 (0.96, 1.08) | | | |
| Hemoglobin (g/L) | 10 | 0.97 (0.88, 1.09) | 0.6412 | | |
| Creatinine (umol/L) | 5 | 0.99 (0.94, 1.03) | 0.5725 | | |
| Uric Acid (umol/L) | 20 | 1.01 (0.97, 1.04) | 0.6206 | | |
| RBC (10¹²/L) | 1 | 1.04 (0.72, 1.50) | 0.8219 | | |
| Total Protein (g/L) | 5 | 1.03 (0.86, 1.22) | 0.7648 | | |
| Lymphocytes (%) | 2 | 1.03 (0.99, 1.07) | 0.1806 | 1.06 (1.01, 1.11) | 0.0203 |
| Potassium (mmol/L) | 1 | 0.80 (0.55, 1.15) | 0.2294 | 0.67 (0.45, 1.01) | 0.0590 |
| Total Bilimbin (umol/L) | 1 | 0.98 (0.95, 1.01) | 0.2908 | | |
| WBC (10⁹/L) | 1 | 1.11 (1.02, 1.20) | 0.0166 | 1.11 (1.01, 1.23) | 0.0330 |
| GGT ≤100 U/L ^{†} | 10 | 1.18 (1.10, 1.26) | <.0001 | 1.12 (1.05, 1.21) | 0.0032 |
| GGT >100 U/L^{†} | 10 | 0.95 (0.92, 0.99) | | 0.95 (0.92, 0.99) | |
| AST >27 U/L & HbA1c >6% | Yes vs. No | 5.20 (3.37, 8.03) | <.0001 | 3.84 (2.40, 6.15) | <.0001 |

| | |
|---|---|
| C-index | 0.7209 |

Results from logistic regression models combined across 10 imputed data sets.
^{†} Non-linear association modeled as linear spline.
^{‡} Non-linear association modeled as quadratic polynomial. Effect sizes for 75th percentile vs. median and for median vs. 25th percentile are presented.
^{#} Non-linear association modeled as cubic polynomial. Effect sizes for 75th percentile vs. median and for median vs. 25th percentile are presented.

Discussion

The current analysis of two independent databases suggests that in patients with imaging evidence of steatosis and/or fibrosis, the combination of serum AST>27 U/L and HbA1c>6% is indicative of NASH with a specificity of approximately 0.9 and positive predictive value around 0.8. Study populations in the two databases were fairly similar with respect to age, race, and ethnicity, although AST was higher in EMMINENCE. As expected, because EMMINENCE patients were clinically selected to participate in a NASH trial a higher proportion of EMMINENCE biopsy-screened patients were found to have NASH diagnosis by biopsy than in the NAFLD Prevalence study (36.6 v. 65.8%). Importantly, AST and HbA1c as continuous variables where the only two variables to come into multivariable models in both studies, with similar magnitudes of association (OR 1.25 v. 1.27 per 5-U/L increment AST, OR 1.81 v. 1.93 per 1% increment HbA1c). Therefore, a second model combining AST and HbA1c was constructed in each database.

The effects of MSDC-0602K among patients with HbA1c specificity >27 U/L and HbA1c>6% were examined. The AST threshold may differentiate NASH in patients at risk, while 6% is a common upper normal limit of HbA1c. Empirical cut-points for AST and HbA1c were quite similar in the two databases and close to those proposed (27 U/L and 6%). Patients with both AST>27 U/L and HbA1c>6% had 3-4 times the odds of having NASH than other patients (adjusted ORs 2.98 and 3.84 in the two studies).

The clinical diagnosis of NASH is difficult and requires liver biopsy. The latter has significant drawbacks including invasiveness leading to some morbidity and even mortality. However, noninvasive tests for the diagnosis of NASH have not been thoroughly assessed, with most available imaging and serological tests directed towards the diagnosis of steatosis and fibrosis.

The findings disclosed herein suggest that in patients with imaging evidence of steatosis and/or fibrosis, simple laboratory tests such as AST and HbA1c may be used for a presumptive diagnosis of NASH. In some cases patients who have NASH may not meet these criteria. However, the high specificity and PPV would suggest that most patients who meet the AST/HbA1c criteria will indeed have NASH.

From the clinical perspective, clinicians caring for patients who have clinical suspicion of NASH should examine the patient's liver enzymes and HbA1c. Those having both AST>27 U/L and HbA1c>6%, should be referred for liver imaging. If such imaging suggests steatosis and/or fibrosis, and other causes of steatohepatitis are excluded, the patient is likely to have NASH. As at this time the treatment options for NASH are limited, liver biopsy may be considered, or the patient can be advised to undertake simple steps suggested to positively affect NASH such as weight loss, diet and possibly vitamin E. Also, if T2D is present and the drug can be tolerated, consideration should be given to the use of pioglitazone. Although, clinicians should bear in mind that the lack of AST>27 U/L and HbA1c>6% does not exclude NASH, and if the clinical suspicion is significant, other imaging and diagnostic takes should be undertaken.

In the context of clinical studies of NASH, liver biopsy has been the cornerstone of NASH diagnosis in almost all studies assessing new therapies for NASH. The need for liver biopsy has been a significant limitation in our ability to recruit large studies of patients with NASH, as few patients would consent to this invasive procedure, and its associated risks. Furthermore, the need for centralized assessment of the liver biopsy makes such studies prohibitively complicated and expensive, again limiting their feasibility and size. In that respect, a simple noninvasive way to select patients with a high likelihood of NASH, without a liver biopsy may enable conducting simple and larger clinical studies of NASH examining different treatment modalities—such as life style modifications, diet and new therapies. Given the high specificity and positive predictive value of combined imaging suggestive of steatosis, inflammation or fibrosis, AST>27 and HbA1c>6%, these criteria are proposed as a simple way to conduct large studies of presumed NASH.

Conclusions

In patients with imaging suggestive of NASH, combined AST>27 U/L and HbA1c>6% have high specificity and positive predictive value for NASH diagnosis. Hence, patients who meet those criteria are highly likely to have NASH. These criteria have low negative predictive value and sensitivity, so patients who do not meet those criteria may still have NASH. Imaging suggestive of steatosis and/or fibrosis combined with AST>27 U/L and HbA1c>6% can be used for entry criteria into large simple studies of patients with presumed NASH.

### Example 9. A Noninvasive Measure of Treatment Response in Nonalcoholic Steatohepatitis—Insights from EMMINENCE and Meta-Analysis

Methods

EMMINENCE was a phase 2 study examining the effects of three doses of MSDC-0602K versus placebo on liver histology in 392 patients with biopsy-confirmed nonalcoholic steatohepatitis (NASH). Patients provided written informed consent, and the protocol and consent form were approved by applicable institutional review boards. Baseline and 12-month biopsies were scored by a single expert hepatopathologist using the NASH Clinical Research Network (CRN) scoring criteria. Fasting blood samples for routine clinical chemistry as well as frozen samples for biomarker assays were analyzed centrally. In this post hoc analysis, multivariable models and partial canonical correlation were used to examine the associations between changes in biomarkers and changes in histological scores from baseline to 12 months in the 339 patients with paired biopsies. The trial was registered on ClinicalTrials.gov (NCT02784444).

Meta-regressions of NASH clinical trials were performed to examine associations between biomarker and histological changes within treatment groups, and to additionally examine associations between treatment effects on biomarkers and on liver histology. Trials that met the following criteria were included:
Randomized controlled clinical trial
Paired biopsies performed at baseline and follow-up, scored using the NASH CRN system.

### NASH Diagnosis

Published results at baseline, follow-up, or changes from baseline for any the following liver injury markers or biomarkers: alanine aminotransferase (ALT), aspartate aminotransferase (AST), alkaline phosphatase, hemoglobin A1c (HbA1c), gamma-glutamyltransferase (GGT), insulin, cytokeratin-18 (CK-18), or Enhanced Liver Fibrosis (ELF) score. Variables selected were based on variables whose change was commonly reported in multiple studies.
Changes in histological scores for ballooning, inflammation, steatosis, and fibrosis, or the pre-determined endpoints of NASH resolution without worsening fibrosis or improvement in fibrosis with no worsening of NASH.

Sample size of at least 20 subjects in each arm of the study.

All results from a search of PubMed for the keywords (“nonalcoholic steatohepatitis” or “non-alcoholic steatohepatitis”) and (“clinical trial” or “randomized”) and filtered for “Clinical Trial” were reviewed. EudraCT and ClinicalTrials.gov were searched for the keyword “nonalcoholic steatohepatitis” with results further filtered for phase 2 or 3, interventional, terminated or completed trials. These three sources returned 265 (PubMed), 153 (ClinicalTrials.gov), and 56 (EudraCT) results, which were each individually reviewed for inclusion in the analysis. Data extracted by one analyst from each source that included means and SDs at baseline, end of study, and changes from baseline to end of study for continuous variables were verified and corrected by a second analyst. Where SDs were not available, any provided estimate of variation was used to derive the SD where possible; a plot-digitizer program was used where data were presented in figures only. Data for dichotomous endpoints were extracted as the number of subjects with the event and corresponding sample size.

All studies included in the current meta-analysis used the NASH CRN histological scoring system for the scoring of the liver biopsy components. The two dichotomous endpoints considered were based on the currently approvable endpoints per FDA guidelines: NASH resolution without worsening of fibrosis, and fibrosis improvement without worsening of NASH.

Statistical Analysis

Multivariable Models

Individual patient data from the 339 subjects with paired biopsy results in EMMINENCE were used to construct univariable and multivariable ANCOVA models for the 12-month change in each histological component (steatosis, inflammation, ballooning, and fibrosis). Candidate predictors included age, sex, type 2 diabetes (T2D) history, and baseline and 12-month changes in weight and laboratory parameters (liver injury markers, cholesterols, insulin sensitivity markers, ELF, CK-18). Non-linearity of the association between each continuous predictor and each outcome was examined by assessing the significance of the non-linear components of a restricted cubic spline transformation for each predictor. Appropriate transformations (quadratic, cubic, linear spline, log 2) were applied where deemed necessary based on review of the Akaike's Information Criterion and visual examination of plots of the predicted outcomes against each predictor. Due to issues with estimating the associations when severe multicollinearity exists between predictors in the model, ALT (collinear with AST) and waist circumference (collinear with weight) were excluded as candidate predictors. Multiple imputation with ten imputed datasets and assuming multivariate normality was used to handle missing values. The occurrence of missing data for any of the predictors was relatively low (˜5% missing one or more predictor values). Rubin's algorithm was used for average estimates for predictors across the imputed datasets. Backwards selection was used for each outcome, with predictors remaining in a majority of the ten imputed datasets at the 0.05 significance level (>5) included in the final model. Each model was adjusted for the baseline value of the outcome. If a change from baseline predictor was included in the final model, then the baseline value for the parameter was also included in the final model.

A second multivariable model was fit for each outcome including the predictors found prognostic for any of the biopsy component changes. A multivariable ANCOVA model that included these predictors was run for NAS (the sum of ballooning, inflammation, and steatosis scores). Multivariable logistic regression models that included these predictors were also run for the binary outcomes of NASH resolution without worsening of fibrosis and fibrosis improvement without worsening of NASH.

Canonical Correlation

A partial canonical correlation using individual patient data from EMMINENCE examined the correlation of changes in weight, AST, HbA1c, GGT, CK-18 (taken together) with changes in ballooning, inflammation, steatosis, and fibrosis (taken together) adjusted for those baseline variables prognostic of any of the histological feature changes in the multivariable models in the 306 subjects with complete observed data for the variables involved. This analysis results in “canonical variates” that are linear combinations of the original sets of variables weighted in such a way as to maximize the correlation between them.

Meta-Regressions

Random effects weighted least squares meta-regressions with inverse variance weighting were used to examine the association of changes in ALT, AST, CK-18, HbA1c, fasting insulin levels, ELF, alkaline phosphatase and GGT with biopsy outcomes. CK-18[M30] was measured in all studies except EMMINENCE where CK-18[M65] was reported. These associations were assessed at an individual cohort level (within treatment group), as well as based on the treatment effect. Percent change from baseline in liver injury makers and biomarkers was used as the predictor when examining the associations in the individual cohorts with outcomes while the treatment ratio, the ratio of active to placebo with respect to the relative changes from baseline, was used as the predictor when assessing the association with the treatment effect on outcomes. Standardized mean changes (mean/SD) or standardized mean treatment differences (mean difference/pooled SD) were used for continuous outcomes and Freeman-Tukey double arcsine transformed proportions or log odds ratios were used for dichotomous outcomes. Meta-regression results were computed using the meta for package available in R. P-values <0.05 were considered statistically significant.

Composite Outcome

From the meta-regression results, ranks for the association with responses and treatment effects for each histologic outcome (changes in ballooning, inflammation, steatosis, and fibrosis; NASH resolution without worsening of fibrosis, and fibrosis improvement without worsening of NAS) were assigned across seven biomarkers (AST, GGT, HbA1c, insulin, CK-18, ELF, and alkaline phosphatase) for both the slope and the mediator p-value (ranks 1-7, with 7 assigned to the biomarker with the greatest slope or lowest p-value). An average rank across all outcomes was then assigned to each biomarker. The three biomarkers with the highest average rank, i.e., with the greatest overall association with the histological outcomes, were then chosen to form a composite outcome. In order to allow comparison of effects across treatment arms and studies, an average, weighted by the biomarker's average rank across all outcomes, was computed for the standardized mean change for each treatment arm and for the standardized mean treatment difference versus placebo for each active treatment arm in each study.

Performance

The value of the resulting novel composite outcome was computed at baseline and 12 months for patients in EMMINENCE. The ability of baseline-adjusted 12-month changes to discriminate between patients with and without NASH resolution without worsening fibrosis, and with and without fibrosis improvement without worsening of NASH, was estimated as the area under the receiver-operator characteristic curve (AUROC) using logistic regression. For comparison, the AUROC was computed similarly for baseline-adjusted changes in other noninvasive measures including ELF, Fibrotest, FIB-4, and Fibroscan® (transient elastography) stiffness measure.

SAS® version 9.4 (SAS Institute, Cary, N.C.) was used where not otherwise stated.

Results

Multivariable Models

Multivariable models developed separately for each biopsy component (ballooning, steatosis, inflammation, and fibrosis) are given in Tables 5-8.

TABLE 5
Univariable and multivariable modeling results for the outcome change from baseline in ballooning

| | | | | | | | | | Multivariable Model | | | |
|---|---|---|---|---|---|---|---|---|---|---|---|---|
| | Univariable Model | | | | Multivariable Model | | | | (All Predictors) | | | |
| | Effect | | | | Effect | | | | Effect | | | |
| | size for | Effect | | | size for | Effect | | | size for | Effect | | |
| | a change | Size | | P- | a change | Size | | P- | a change | Size | | P- |
| Parameter | of: | (95% CI) | | Value | of: | (95% CI) | | Value | of: | (95% CI) | | Value |
| Baseline | | | | | | | | | | | | |
| Variables | | | | | | | | | | | | |
| Age, years | 57.00 vs. | 0.01 | (−0.05, 0.06) | 0.1679 | 57.00 vs. | 0.04 | (−0.01, 0.10) | 0.0036 | 57.00 vs. | 0.04 | (−0.03, 0.10) | 0.0077 |
| | 50.00 | | | | 50.00 | | | | 50.00 | | | |
| | 65.00 vs. | −0.07 | (−0.17, 0.02) | | 65.00 vs. | −0.03 | (−0.12, 0.06) | . | 65.00 vs. | −0.04 | (−0.14, 0.06) | . |
| | 57.00 | | | | 57.00 | | | | 57.00 | | | |
| Male Sex | Yes vs. | −0.04 | (−0.22, 0.14) | 0.6883 | | | | | | | | |
| | No | | | | | | | | | | | |
| Diabetes | Yes vs. | 0.14 | (−0.04, 0.32) | 0.1400 | | | | | Yes vs. | −0.05 | (−0.26, 0.16) | 0.6419 |
| | No | | | | | | | | No | | | |
| Weight, kg | 5 | −0.00 | (−0.02, 0.02) | 0.9678 | | | | | 5 | 0.00 | (−0.02, 0.02) | 0.8572 |
| Waist | 10 | 0.01 | (−0.06, 0.07) | 0.7923 | | | | | | | | |
| Circumfer- | | | | | | | | | | | | |
| ence, cm | | | | | | | | | | | | |
| Albumin, | 1 | −0.03 | (−0.06, 0.01) | 0.1275 | | | | | | | | |
| g/L | | | | | | | | | | | | |
| Alk. Phos, | Doubling | 0.01 | (−0.19, 0.21) | 0.9094 | | | | | | | | |
| U/L | | | | | | | | | | | | |
| ALT, U/L | Doubling | 0.20 | (0.08, 0.31) | 0.0008 | | | | | | | | |
| AST, U/L | Doubling | 0.28 | (0.14, 0.42) | 0.0002 | Doubling | 0.33 | (0.14, 0.52) | 0.0007 | Doubling | 0.30 | (0.09, 0.50) | 0.0047 |
| AST/ALT | 1 | −0.13 | (−0.46, 0.21) | 0.4584 | | | | | | | | |
| Ratio | | | | | | | | | | | | |
| Bilirubin, | 1 | −0.00 | (−0.02, 0.02) | 0.9807 | | | | | | | | |
| umol/L | | | | | | | | | | | | |
| Triglyc- | Doubling | 0.16 | (0.04, 0.28) | 0.0109 | Doubling | 0.10 | (−0.02, 0.21) | 0.0917 | Doubling | 0.09 | (−0.02, 0.21) | 0.1174 |
| erides, | | | | | | | | | | | | |
| mmol/L | | | | | | | | | | | | |
| Cholesterol, | 1 | 0.02 | (−0.07, 0.10) | 0.7082 | | | | | | | | |
| mmol/L | | | | | | | | | | | | |
| HDL, | 1 | −0.07 | (−0.34, 0.20) | 0.6184 | | | | | | | | |
| mmol/L | | | | | | | | | | | | |
| LDL, | 1 | −0.03 | (−0.12, 0.06) | 0.5199 | | | | | | | | |
| mmol/L | | | | | | | | | | | | |
| Glucose, | 1 | 0.05 | (0.01, 0.09) | 0.0111 | | | | | | | | |
| mmol/L | | | | | | | | | | | | |
| HbA1c, % | 0.25 | 0.04 | (0.02, 0.06) | 0.0006 | 0.25 | 0.03 | (0.01, 0.05) | 0.0030 | 0.25 | 0.04 | (0.01, 0.06) | 0.0103 |
| GGT, U/L | 20 | 0.03 | (0.01, 0.06) | 0.0199 | | | | | 10 | 0.01 | (−0.01, 0.02) | 0.3681 |
| Insulin, | 10 | 0.06 | (0.02, 0.09) | 0.0014 | 10 | 0.05 | (0.02, 0.08) | 0.0043 | 5 | 0.02 | (0.01, 0.04) | 0.0066 |
| uIU/mL | | | | | | | | | | | | |
| INR, Ratio | 0.1 | −0.04 | (−0.15, 0.07) | 0.4906 | | | | | | | | |
| Platelets, | 20 | −0.01 | (−0.04, 0.01) | 0.3087 | | | | | | | | |
| GI/L | | | | | | | | | | | | |
| ELF | 1 | 0.12 | (0.03, 0.22) | 0.0113 | | | | | 1 | 0.02 | (−0.09, 0.13) | 0.7442 |
| CYFRA-18, | 100 | 0.03 | (0.01, 0.05) | 0.0007 | 100 | 0.02 | (−0.00, 0.04) | 0.0772 | 100 | 0.02 | (−0.00, 0.04) | 0.0942 |
| U/L | | | | | | | | | | | | |
| Change from | | | | | | | | | | | | |
| Baseline | | | | | | | | | | | | |
| Variables: | | | | | | | | | | | | |
| Weight, kg | 5 | 0.05 | (−0.02, 0.13) | 0.1665 | | | | | 5 | 0.02 | (−0.05, 0.09) | 0.5627 |
| Waist | 10 | 0.01 | (−0.12, 0.15) | 0.8314 | | | | | | | | |
| Circumfer- | | | | | | | | | | | | |
| ence, cm | | | | | | | | | | | | |
| Albumin, | 1 | −0.01 | (−0.04, 0.03) | 0.7171 | | | | | | | | |
| g/L | | | | | | | | | | | | |
| Alk. Phos, | 5 | 0.02 | (−0.00, 0.05) | 0.1085 | | | | | | | | |
| U/L | | | | | | | | | | | | |
| ALT, U/L | 5 | 0.02 | (0.00, 0.04) | 0.0324 | | | | | | | | |
| AST, U/L | 5 | 0.03 | (0.01, 0.05) | 0.0098 | 5 | 0.02 | (−0.01, 0.05) | 0.0817 | 5 | 0.02 | (−0.01, 0.05) | 0.0987 |
| AST/ALT | 1 | 0.23 | (−0.17, 0.64) | 0.2607 | | | | | | | | |
| Ratio | | | | | | | | | | | | |
| Bilirubin, | 1 | 0.02 | (−0.01, 0.04) | 0.3090 | | | | | | | | |
| umol/L | | | | | | | | | | | | |
| Triglyc- | 1 | −0.05 | (−0.12, 0.02) | 0.1420 | | | | | | | | |
| erides, | | | | | | | | | | | | |
| mmol/L | | | | | | | | | | | | |
| Cholesterol, | 1 | −0.05 | (−0.15, 0.05) | 0.3013 | | | | | | | | |
| mmol/L | | | | | | | | | | | | |
| HDL, | 1 | −0.10 | (−0.58, 0.38) | 0.6855 | | | | | | | | |
| mmol/L | | | | | | | | | | | | |
| LDL, | 1 | 0.00 | (−0.10, 0.11) | 0.9540 | | | | | | | | |
| mmol/L | | | | | | | | | | | | |
| Glucose, | 1 | 0.05 | (0.01, 0.09) | 0.0075 | | | | | | | | |
| mmol/L | | | | | | | | | | | | |
| HbA1c, % | 0.25 | 0.04 | (0.01, 0.06) | 0.0037 | 0.25 | 0.03 | (0.01, 0.06) | 0.0132 | 0.25 | 0.03 | (0.01, 0.06) | 0.0175 |
| GGT, U/L | 10 | 0.02 | (−0.01, 0.04) | 0.1257 | | | | | 10 | 0.01 | (−0.02, 0.04) | 0.6157 |
| Insulin, | 5 | −0.01 | (−0.03, 0.01) | 0.2536 | | | | | | | | |
| uIU/mL | | | | | | | | | | | | |
| INR, Ratio | 0.1 | 0.07 | (−0.04, 0.19) | 0.2181 | | | | | | | | |
| Platelets, | 20 | −0.00 | (−0.04, 0.04) | 0.9996 | | | | | | | | |
| GI/L | | | | | | | | | | | | |
| ELF | 1 | 0.07 | (−0.06, 0.20) | 0.2727 | | | | | | | | |
| CYFRA-18, | 100 | 0.02 | (0.01, 0.04) | 0.0007 | 100 | 0.02 | (0.00, 0.03) | 0.0201 | 100 | 0.02 | (0.00, 0.04) | 0.0246 |
| U/L | | | | | | | | | | | | |
| MSDC- | 20 | −0.01 | (−0.03, 0.01) | 0.2192 | 20 | −0.02 | (−0.03, 0.02) | 0.2897 | | | | |
| 0602K | | | | | | | | | | | | |
| Dose | | | | | | | | | | | | |

| | |
|---|---|
| Adjusted R²: 0.3942 | Adjusted R²: 0.3850 |
| Adjusted R2 w/Dose: 0.3926 | |

TABLE 6
Univariable and multivariable modeling results for the outcome change from baseline in steatosis

| | | | | | | | | | Multivariable Model | | | |
|---|---|---|---|---|---|---|---|---|---|---|---|---|
| | Univariable Model | | | | Multivariable Model | | | | (All Predictors) | | | |
| | Effect | | | | Effect | | | | Effect | | | |
| | size for | Effect | | | size for | Effect | | | size for | Effect | | |
| | a change | Size | | P- | a change | Size | | P- | a change | Size | | P- |
| Parameter | of: | (95% CI) | | Value | of: | (95% CI) | | Value | of: | (95% CI) | | Value |
| Baseline | | | | | | | | | | | | |
| Variables: | | | | | | | | | | | | |
| Age, years | 5 | −0.05 | (−0.09, −0.01) | 0.0086 | 5 | −0.04 | (−0.08, 0.00) | 0.0664 | 5 | −0.06 | (−0.11, −0.02) | 0.0072 |
| Male Sex | Yes vs. | 0.06 | (−0.11, 0.23) | 0.4894 | | | | | | | | |
| | No | | | | | | | | | | | |
| Diabetes | Yes vs. | 0.12 | (−0.05, 0.29) | 0.1555 | | | | | Yes vs. | 0.08 | (−0.13, 0.28) | 0.4508 |
| | No | | | | | | | | No | | | |
| Weight, kg | 99.06 vs. | 0.08 | (0.01, 0.16) | 0.0550 | 99.06 vs. | 0.06 | (−0.01, 0.13) | 0.1038 | 99.06 vs. | 0.06 | (−0.01, 0.13) | 0.1240 |
| | 85.91 | | | | 85.91 | | | | 85.91 | | | |
| | 113.40 vs. | 0.03 | (−0.03, 0.09) | . | 113.40 vs. | 0.00 | (−0.06, 0.07) | . | 113.40 vs. | 0.00 | (−0.06, 0.07) | . |
| | 99.06 | | | | 99.06 | | | | 99.06 | | | |
| Waist | 113.03 vs. | 0.02 | (−0.05, 0.09) | 0.5891 | | | | | | | | |
| Circumfer- | 104.50 | | | | | | | | | | | |
| ence, cm | | | | | | | | | | | | |
| | 123.00 vs. | −0.01 | (−0.07, 0.06) | . | | | | | | | | |
| | 113.03 | | | | | | | | | | | |
| Albumin, | 1 | 0.01 | (−0.02, 0.04) | 0.5648 | | | | | | | | |
| g/L | | | | | | | | | | | | |
| Alk. Phos, | 5 | −0.01 | (−0.03, 0.00) | 0.1131 | | | | | | | | |
| U/L | | | | | | | | | | | | |
| ALT, U/L | Doubling | 0.05 | (−0.06, 0.16) | 0.3718 | | | | | | | | |
| AST, U/L | Doubling | −0.01 | (−0.15, 0.12) | 0.8404 | Doubling | 0.10 | (−0.01, 0.21) | 0.0914 | Doubling | 0.08 | (−0.01, 0.21) | 0.1017 |
| AST/ALT | 1 | −0.31 | (−0.62, 0.00) | 0.0517 | | | | | | | | |
| Ratio | | | | | | | | | | | | |
| Bilirubin, | 1 | −0.00 | (−0.02, 0.02) | 0.9732 | | | | | | | | |
| umol/L | | | | | | | | | | | | |
| Triglyc- | 1 | −0.02 | (−0.08, 0.03) | 0.3593 | | | | | 1 | −0.02 | (−0.07, 0.03) | 0.4261 |
| erides, | | | | | | | | | | | | |
| mmol/L | | | | | | | | | | | | |
| Cholesterol, | 4.81 vs. | 0.01 | (−0.05, 0.08) | 0.0001 | | | | | | | | |
| mmol/L | 4.14 | | | | | | | | | | | |
| | 5.48 vs. | 0.11 | (0.03, 0.18) | | | | | | | | | |
| | 4.81 | | | | | | | | | | | |
| HDL, | 1 | −0.00 | (− 0.26, 0.25) | 0.9786 | | | | | | | | |
| mmol/L | | | | | | | | | | | | |
| LDL, | 1 | −0.00 | (− 0.09, 0.08) | 0.9731 | | | | | | | | |
| mmol/L | | | | | | | | | | | | |
| Glucose, | 1 | 0.03 | (−0.01, 0.06) | 0.1326 | | | | | | | | |
| mmol/L | | | | | | | | | | | | |
| HbA1c, % | 0.25 | 0.01 | (−0.01, 0.03) | 0.1730 | 0.25 | 0.02 | (0.00, 0.04) | 0.0314 | 0.25 | 0.02 | (−0.00, 0.04) | 0.0759 |
| GGT, U/L | 43.00 vs. | −0.02 | (−0.05, 0.01) | 0.6461 | 43.00 vs. | −0.01 | (−0.04, 0.03) | 0.8600 | 43.00 vs. | −0.01 | (−0.04, 0.03) | 0.8843 |
| | 31.00 | | | | 31.00 | | | | 31.00 | | | |
| | 71.00 vs. | −0.04 | (−0.11, 0.03) | | 71.00 vs. | −0.01 | (−0.09, 0.06) | . | 71.00 vs. | −0.02 | (−0.09, 0.06) | |
| | 43.00 | | | | 43.00 | | | | 43.00 | | | |
| Insulin, | 10 | 0.01 | (−0.03, 0.04) | 0.6681 | | | | | 5 | −0.00 | (−0.02, 0.01) | 0.7663 |
| uIU/mL | | | | | | | | | | | | |
| INR, Ratio | 0.1 | −0.02 | (−0.12, 0.09) | 0.7665 | | | | | | | | |
| Platelets, | 238.00 vs. | −0.04 | (−0.11, 0.02) | 0.2857 | | | | | | | | |
| GI/L | 200.00 | | | | | | | | | | | |
| | 285.00 vs. | −0.00 | (−0.06, 0.06) | | | | | | | | | |
| | 238.00 | | | | | | | | | | | |
| ELF | 1 | 0.02 | (−0.07, 0.11) | 0.6324 | | | | | 1 | 0.07 | (−0.03, 0.18) | 0.1718 |
| CYFRA-18, | 100 | 0.00 | (−0.01, 0.02) | 0.8344 | | | | | 100 | 0.01 | (−0.01, 0.03) | 0.5714 |
| U/L | | | | | | | | | | | | |
| Change from | | | | | | | | | | | | |
| Baseline | | | | | | | | | | | | |
| Variables: | | | | | | | | | | | | |
| Weight, kg | 5 | 0.10 | (0.03, 0.17) | 0.0063 | 5 | 0.09 | (0.02, 0.15) | 0.0146 | 5 | 0.08 | (0.01, 0.15) | 0.0210 |
| Waist | 10 | 0.09 | (−0.04, 0.22) | 0.1588 | | | | | | | | |
| Circumfer- | | | | | | | | | | | | |
| ence, cm | | | | | | | | | | | | |
| Albumin, | 1 | 0.01 | (−0.03, 0.04) | 0.6892 | | | | | | | | |
| g/L | | | | | | | | | | | | |
| Alk. Phos, | 5 | 0.03 | (0.00, 0.05) | 0.0489 | | | | | | | | |
| U/L | | | | | | | | | | | | |
| ALT, U/L | 5 | 0.04 | (0.02, 0.05) | <.0001 | | | | | | | | |
| AST, U/L | 5 | 0.05 | (0.03, 0.06) | <.0001 | 5 | 0.02 | (0.00, 0.05) | 0.0366 | 5 | 0.02 | (−0.01, 0.05) | 0.1128 |
| AST/ALT | 1 | −0.37 | (−0.76, 0.01) | 0.0592 | | | | | | | | |
| Ratio | | | | | | | | | | | | |
| Bilirubin, | 1 | 0.01 | (−0.02, 0.03) | 0.6331 | | | | | | | | |
| umol/L | | | | | | | | | | | | |
| Triglyc- | 1 | 0.06 | (−0.00, 0.13) | 0.0555 | | | | | | | | |
| erides, | | | | | | | | | | | | |
| mmol/L | | | | | | | | | | | | |
| Cholesterol, | 1 | 0.01 | (−0.08, 0.11) | 0.7580 | | | | | | | | |
| mmol/L | | | | | | | | | | | | |
| HDL, | 1 | −0.47 | (−0.92, −0.02) | 0.0404 | | | | | | | | |
| mmol/L | | | | | | | | | | | | |
| LDL, | 1 | −0.02 | (−0.12, 0.07) | 0.6259 | | | | | | | | |
| mmol/L | | | | | | | | | | | | |
| Glucose, | 1 | 0.05 | (0.02, 0.09) | 0.0025 | | | | | | | | |
| mmol/L | | | | | | | | | | | | |
| HbA1c, % | 0.25 | 0.05 | (0.02, 0.07) | <.0001 | 0.25 | 0.02 | (−0.00, 0.05) | 0.0654 | 0.25 | 0.03 | (−0.00, 0.05) | 0.0574 |
| GGT, U/L | 10 | 0.05 | (0.03, 0.08) | <.0001 | 10 | 0.03 | (0.01, 0.06) | 0.0161 | 10 | 0.03 | (0.00, 0.06) | 0.0336 |
| Insulin, | 5 | 0.01 | (−0.01, 0.03) | 0.4752 | | | | | | | | |
| uIU/mL | | | | | | | | | | | | |
| INR, Ratio | 0.1 | 0.03 | (−0.08, 0.15) | 0.5511 | | | | | | | | |
| Platelets, | 20 | −0.01 | (−0.05, 0.03) | 0.5908 | | | | | | | | |
| GI/L | | | | | | | | | | | | |
| ELF | 1 | 0.05 | (−0.07, 0.17) | 0.4404 | | | | | | | | |
| CYFRA-18, | 100 | 0.02 | (0.01, 0.04) | 0.0001 | | | | | 100 | 0.01 | (−0.01, 0.02) | 0.4726 |
| U/L | | | | | | | | | | | | |
| MSDC- | 20 | −0.03 | (−0.05, −0.01) | 0.0040 | | −0.03 | (−0.05, −0.01) | 0.0031 | | | | |
| 0602K | | | | | | | | | | | | |
| Dose | | | | | | | | | | | | |

| | |
|---|---|
| Adjusted R²: 0.3422 | Adjusted R²: 0.3942 |
| Adjusted R² w/Dose: 0.3583 | |

TABLE 7
Univariable and multivariable modeling results for the outcome change from baseline in inflammation

| | | | | | | | | | Multivariable Model | | | |
|---|---|---|---|---|---|---|---|---|---|---|---|---|
| | Univariable Model | | | | Multivariable Model | | | | (All Predictors) | | | |
| | Effect | | | | Effect | | | | Effect | | | |
| | size for | Effect | | | size for | Effect | | | size for | Effect | | |
| | a change | Size | | P- | a change | Size | | P- | a change | Size | | P- |
| Parameter | of: | (95% CI) | | Value | of: | (95% CI) | | Value | of: | (95% CI) | | Value |
| Baseline | | | | | | | | | | | | |
| Variables: | | | | | | | | | | | | |
| Age, years | 5 | −0.04 | (−0.06, −0.01) | 0.0076 | 5 | −0.04 | (−0.07, −0.01) | 0.0076 | 5 | −0.04 | (−0.07, −0.01) | 0.0059 |
| Male Sex | Yes vs. | 0.08 | (−0.04, 0.19) | 0.1968 | | | | | | | | |
| | No | | | | | | | | | | | |
| Diabetes | Yes vs. | 0.05 | (−0.07, 0.16) | 0.4489 | | | | | Yes vs. | 0.05 | (−0.09, 0.18) | 0.5022 |
| | No | | | | | | | | No | | | |
| Weight, kg | 5 | 0.00 | (−0.01, 0.02) | 0.8272 | | | | | 5 | −0.00 | (−0.02, 0.01) | 0.5427 |
| Waist | 10 | −0.00 | (−0.05, 0.04) | 0.8451 | | | | | | | | |
| Circumfer- | | | | | | | | | | | | |
| ence, cm | | | | | | | | | | | | |
| Albumin, | 45.00 vs. | −0.02 | (−0.06, 0.03) | 0.5795 | | | | | | | | |
| g/L | 43.00 | | | | | | | | | | | |
| | 47.00 vs. | 0.02 | (−0.04, 0.07) | | | | | | | | | |
| | 45.00 | | | | | | | | | | | |
| Alk. Phos, | 5 | 0.00 | (−0.01, 0.01) | 0.6672 | | | | | | | | |
| U/L | | | | | | | | | | | | |
| ALT, U/L | Doubling | 0.12 | (0.05, 0.20) | 0.0015 | | | | | | | | |
| AST, U/L | Doubling | 0.15 | (0.06, 0.24) | 0.0014 | Doubling | 0.11 | (−0.02, 0.23) | 0.0922 | Doubling | 0.11 | (−0.02, 0.24) | 0.0938 |
| AST/ALT | 1 | −0.11 | (−0.32, 0.11) | 0.3272 | | | | | | | | |
| Ratio | | | | | | | | | | | | |
| Bilirubin, | 1 | 0.00 | (−0.01, 0.01) | 0.7428 | | | | | | | | |
| umol/L | | | | | | | | | | | | |
| Triglyc- | 1 | 0.01 | (−0.02, 0.05) | 0.4780 | | | | | 1 | 0.01 | (−0.03, 0.04) | 0.6665 |
| erides, | | | | | | | | | | | | |
| mmol/L | | | | | | | | | | | | |
| Cholesterol, | 1 | −0.01 | (−0.06, 0.05) | 0.7514 | | | | | | | | |
| mmol/L | | | | | | | | | | | | |
| HDL, | 1 | −0.04 | (−0.22, 0.13) | 0.6176 | | | | | | | | |
| mmol/L | | | | | | | | | | | | |
| LDL, | 1 | −0.02 | (−0.08, 0.04) | 0.4878 | | | | | | | | |
| mmol/L | | | | | | | | | | | | |
| Glucose, | 1 | 0.03 | (0.00, 0.05) | 0.0377 | | | | | | | | |
| mmol/L | | | | | | | | | | | | |
| HbA1c, % | 0.25 | 0.01 | (−0.00, 0.02) | 0.1932 | 0.25 | 0.01 | (−0.01, 0.02) | 0.2341 | 0.25 | 0.00 | (−0.01, 0.02) | 0.6199 |
| GGT, U/L | 10 | 0.01 | (−0.00, 0.01) | 0.1832 | | | | | 10 | −0.00 | (−0.01, 0.01) | 0.8161 |
| Insulin, | 5 | 0.02 | (0.01, 0.03) | 0.0016 | 10 | 0.03 | (0.00, 0.05) | 0.0179 | 5 | 0.01 | (0.00, 0.02) | 0.0252 |
| uIU/mL | | | | | | | | | | | | |
| INR, Ratio | 0.1 | −0.03 | (−0.11, 0.04) | 0.3691 | | | | | | | | |
| Platelets, | 20 | −0.01 | (−0.03, 0.00) | 0.1047 | | | | | | | | |
| GI/L | | | | | | | | | | | | |
| ELF | 1 | 0.07 | (0.01, 0.14) | 0.0173 | 1 | 0.08 | (0.02, 0.15) | 0.0172 | 1 | 0.09 | (0.02, 0.16) | 0.0149 |
| CYFRA-18, | 100 | 0.02 | (0.01, 0.03) | <.0001 | 100 | 0.02 | (0.01, 0.03) | 0.0054 | 100 | 0.02 | (0.01, 0.03) | 0.0061 |
| U/L | | | | | | | | | | | | |
| Change from | | | | | | | | | | | | |
| Baseline | | | | | | | | | | | | |
| Variables | | | | | | | | | | | | |
| Weight, kg | 5 | −0.00 | (−0.05, 0.05) | 0.9487 | | | | | 5 | −0.01 | (−0.06, 0.04) | 0.6919 |
| Waist | 10 | 0.00 | (−0.09, 0.09) | 0.9982 | | | | | | | | |
| Circumfer- | | | | | | | | | | | | |
| ence, cm | | | | | | | | | | | | |
| Albumin, | 1 | 0.02 | (−0.00, 0.04) | 0.1243 | | | | | | | | |
| g/L | | | | | | | | | | | | |
| Alk. Phos, | 5 | 0.03 | (0.01, 0.04) | 0.0019 | | | | | | | | |
| U/L | | | | | | | | | | | | |
| ALT, U/L | 5 | 0.01 | (0.00, 0.02) | 0.0314 | | | | | | | | |
| AST, U/L | 5 | 0.02 | (0.01, 0.04) | 0.0003 | 5 | 0.02 | (0.00, 0.04) | 0.0324 | 5 | 0.02 | (0.00, 0.04) | 0.0249 |
| AST/ALT | 1 | 0.18 | (−0.08, 0.45) | 0.1809 | | | | | | | | |
| Ratio | | | | | | | | | | | | |
| Bilirubin, | 1 | 0.01 | (−0.00, 0.03) | 0.1305 | | | | | | | | |
| umol/L | | | | | | | | | | | | |
| Triglyc- | 1 | −0.01 | (−0.05, 0.03) | 0.6395 | | | | | | | | |
| erides, | | | | | | | | | | | | |
| mmol/L | | | | | | | | | | | | |
| Cholesterol, | 1 | 0.02 | (−0.04, 0.08) | 0.5504 | | | | | | | | |
| mmol/L | | | | | | | | | | | | |
| HDL, | 1 | −0.29 | (−0.61, 0.03) | 0.0750 | | | | | | | | |
| mmol/L | | | | | | | | | | | | |
| LDL, | 1 | 0.04 | (−0.02, 0.11) | 0.2007 | | | | | | | | |
| mmol/L | | | | | | | | | | | | |
| Glucose, | 1 | 0.02 | (−0.00, 0.05) | 0.0550 | | | | | | | | |
| mmol/L | | | | | | | | | | | | |
| HbA1c, % | 0.25 | 0.03 | (0.02, 0.05) | <.0001 | 0.25 | 0.03 | (0.01, 0.04) | 0.0020 | 0.25 | 0.03 | (0.01, 0.04) | 0.0027 |
| GGT, U/L | 10 | 0.01 | (−0.00, 0.03) | 0.1235 | | | | | 10 | −0.01 | (−0.02, 0.01) | 0.5029 |
| Insulin, | 5 | 0.00 | (−0.01, 0.02) | 0.7769 | | | | | | | | |
| uIU/mL | | | | | | | | | | | | |
| INR, Ratio | 0.1 | 0.04 | (−0.04, 0.12) | 0.2887 | | | | | | | | |
| Platelets, | 20 | 0.01 | (−0.02, 0.04) | 0.4836 | | | | | | | | |
| GI/L | | | | | | | | | | | | |
| ELF | 1 | 0.03 | (−0.06, 0.11) | 0.5093 | | | | | | | | |
| CYFRA-18, | 100 | 0.02 | (0.01, 0.03) | 0.0001 | 100 | 0.01 | (−0.00, 0.02) | 0.0873 | 100 | 0.01 | (−0.00, 0.02) | 0.0821 |
| U/L | | | | | | | | | | | | |
| MSDC- | 20 | −0.01 | (−0.02, 0.00) | 0.2237 | 20 | 0.01 | (−0.01, 0.03) | 0.3718 | | | | |
| 0602K | | | | | | | | | | | | |
| Dose | | | | | | | | | | | | |

| | |
|---|---|
| Adjusted R²: 0.4775 | Adjusted R²: 0.4704 |
| Adjusted R² w/Dose: 0.4772 | |

TABLE 8
Univariable and multivariable modeling results for the outcome change from baseline in fibrosis

| | | | | | | | | | Multivariable Model | | | |
|---|---|---|---|---|---|---|---|---|---|---|---|---|
| | Univariable Model | | | | Multivariable Model | | | | (All Predictors) | | | |
| | Effect | | | | Effect | | | | Effect | | | |
| | size for | Effect | | | size for | Effect | | | size for | Effect | | |
| | a change | Size | | P- | a change | Size | | P- | a change | Size | | P- |
| Parameter | of: | (95% CI) | | Value | of: | (95% CI) | | Value | of: | (95% CI) | | Value |
| Baseline | | | | | | | | | | | | |
| Variables: | | | | | | | | | | | | |
| Age, years | 5 | 0.03 | (−0.02, 0.08) | 0.2670 | | | | | 5 | 0.03 | (−0.03, 0.08) | 0.3475 |
| Male Sex | Yes vs. | 0.00 | (−0.22, 0.23) | 0.9805 | | | | | | | | |
| | No | | | | | | | | | | | |
| Diabetes | Yes vs. | 0.27 | (0.05, 0.49) | 0.0190 | Yes vs. | 0.24 | (0.04, 0.44) | 0.0196 | Yes vs. | 0.21 | (−0.04, 0.46) | 0.0940 |
| | No | | | | No | | | | No | | | |
| Weight, kg | 5 | −0.00 | (−0.03, 0.03) | 0.8605 | | | | | 5 | 0.01 | (−0.02, 0.03) | 0.5134 |
| Waist | 10 | 0.04 | (−0.04, 0.12) | 0.3825 | | | | | | | | |
| Circumfer- | | | | | | | | | | | | |
| ence, cm | | | | | | | | | | | | |
| Albumin, | 45.00 vs. | −0.00 | (−0.09, 0.09) | 0.9806 | | | | | | | | |
| g/L | 43.00 | | | | | | | | | | | |
| | 47.00 vs. | −0.01 | (−0.11, 0.09) | | | | | | | | | |
| | 45.00 | | | | | | | | | | | |
| Alk. Phos, | 5 | −0.01 | (−0.03, 0.01) | 0.3443 | | | | | | | | |
| U/L | | | | | | | | | | | | |
| ALT, U/L | Doubling | 0.01 | (0.00, 0.01) | 0.0016 | | | | | | | | |
| AST, U/L | Doubling | 0.04 | (0.02, 0.06) | 0.0007 | Doubling | 0.51 | (0.27, 0.74) | <.0001 | Doubling | 0.51 | (0.26, 0.76) | <.0001 |
| AST/ALT | 0.82 vs. | −0.03 | (−0.12, 0.06) | 0.4756 | | | | | | | | |
| Ratio | 0.67 | | | | | | | | | | | |
| | 1.00 vs. | 0.01 | (−0.07, 0.09) | | | | | | | | | |
| | 0.82 | | | | | | | | | | | |
| Bilirubin, | 1 | 0.01 | (−0.01, 0.03) | 0.3620 | | | | | | | | |
| umol/L | | | | | | | | | | | | |
| Triglyc- | 1 | 0.29 | (0.08, 0.49) | 0.0157 | | | | | 1 | 0.19 | (0.00, 0.37) | 0.1345 |
| erides, | | | | | | | | | | | | |
| mmol/L | | | | | | | | | | | | |
| | 1 | −0.07 | (−0.15, 0.02) | | | | | | 1 | −0.04 | (−0.12, 0.04) | |
| Cholesterol, | 1 | −0.03 | (−0.13, 0.07) | 0.5579 | | | | | | | | |
| mmol/L | | | | | | | | | | | | |
| HDL, | 1 | −0.16 | (−0.50, 0.17) | 0.3296 | | | | | | | | |
| mmol/L | | | | | | | | | | | | |
| LDL, | 1 | −0.03 | (−0.14, 0.08) | 0.5941 | | | | | | | | |
| mmol/L | | | | | | | | | | | | |
| Glucose, | 1 | 0.04 | (−0.00, 0.09) | 0.0697 | | | | | | | | |
| mmol/L | | | | | | | | | | | | |
| HbA1c, % | 0.25 | 0.04 | (0.01, 0.06) | 0.0059 | | | | | 0.25 | 0.00 | (−0.03, 0.03) | 0.8557 |
| GGT, U/L | 10 | 0.03 | (0.01, 0.05) | 0.0004 | 10 | 0.02 | (0.01, 0.04) | 0.0095 | 10 | 0.01 | (−0.00, 0.03) | 0.1607 |
| Insulin, | 5 | 0.02 | (−0.01, 0.04) | 0.1824 | | | | | 5 | 0.00 | (−0.02, 0.02) | 0.8620 |
| uIU/mL | | | | | | | | | | | | |
| INR, Ratio | 0.1 | 0.11 | (−0.02, 0.25) | 0.1058 | | | | | | | | |
| Platelets, | 20 | −0.01 | (−0.05, 0.02) | 0.3712 | | | | | | | | |
| GI/L | | | | | | | | | | | | |
| ELF | 1 | 0.28 | (0.16, 0.40) | <.0001 | 1 | 0.19 | (0.08, 0.30) | 0.0008 | 1 | 0.14 | (0.01, 0.28) | 0.0332 |
| CYFRA-18, | 100 | 0.10 | (0.06, 0.14) | <.0001 | 100 | 0.04 | (0.00, 0.09) | 0.1086 | 100 | 0.05 | (0.00, 0.09) | 0.1060 |
| U/L | | | | | | | | | | | | |
| | 100 | −0.02 | (−0.06, 0.03) | | 100 | −0.03 | (−0.06, 0.01) | | 100 | −0.02 | (−0.06, 0.02) | |
| Change from | | | | | | | | | | | | |
| Baseline | | | | | | | | | | | | |
| Variables: | | | | | | | | | | | | |
| Weight, kg | 5 | 0.05 | (−0.04, 0.15) | 0.2683 | | | | | 5 | 0.03 | (−0.05, 0.12) | 0.4779 |
| Waist | 10 | 0.02 | (−0.14, 0.19) | 0.7763 | | | | | | | | |
| Circumfer- | | | | | | | | | | | | |
| ence, cm | | | | | | | | | | | | |
| Albumin, | 1 | 0.00 | (−0.04, 0.05) | 0.9531 | | | | | | | | |
| g/L | | | | | | | | | | | | |
| Alk. Phos, | 5 | 0.04 | (0.01, 0.07) | 0.0171 | | | | | | | | |
| U/L | | | | | | | | | | | | |
| ALT, U/L | 5 | 0.03 | (0.01, 0.05) | 0.0126 | | | | | | | | |
| AST, U/L | 5 | 0.04 | (0.02, 0.07) | 0.0012 | 5 | 0.08 | (0.06, 0.11) | <.0001 | 5 | 0.08 | (0.04, 0.11) | <.0001 |
| AST/ALT | 1 | 0.14 | (−0.37, 0.64) | 0.5899 | | | | | | | | |
| Ratio | | | | | | | | | | | | |
| Bilirubin, | 1 | 0.00 | (−0.04, 0.04) | 0.9464 | | | | | | | | |
| umol/L | | | | | | | | | | | | |
| Triglyc- | 1 | −0.00 | (−0.08, 0.08) | 0.9919 | | | | | | | | |
| erides, | | | | | | | | | | | | |
| mmol/L | | | | | | | | | | | | |
| Cholesterol, | 1 | 0.02 | (−0.10, 0.14) | 0.7305 | | | | | | | | |
| mmol/L | | | | | | | | | | | | |
| HDL, | 1 | −0.01 | (−0.59, 0.58) | 0.9771 | | | | | | | | |
| mmol/L | | | | | | | | | | | | |
| LDL, | 1 | 0.02 | (−0.10, 0.15) | 0.7192 | | | | | | | | |
| mmol/L | | | | | | | | | | | | |
| Glucose, | 1 | 0.04 | (−0.01, 0.09) | 0.0859 | | | | | | | | |
| mmol/L | | | | | | | | | | | | |
| HbA1c, % | 0.25 | 0.03 | (−0.00, 0.06) | 0.0515 | | | | | 0.25 | 0.02 | (−0.02, 0.05) | 0.3413 |
| GGT, U/L | 10 | 0.01 | (−0.02, 0.04) | 0.4828 | | | | | 10 | −0.01 | (−0.04, 0.03) | 0.7059 |
| Insulin, | 5 | 0.01 | (−0.02, 0.04) | 0.5134 | | | | | | | | |
| uIU/mL | | | | | | | | | | | | |
| INR, Ratio | 0.1 | 0.07 | (−0.08, 0.21) | 0.3574 | | | | | | | | |
| Platelets, | 20 | 0.01 | (−0.04, 0.06) | 0.7006 | | | | | | | | |
| GI/L | | | | | | | | | | | | |
| ELF | 1 | −0.02 | (−0.18, 0.14) | 0.8321 | | | | | | | | |
| CYFRA-18, | 100 | 0.02 | (0.01, 0.04) | 0.0074 | | | | | 100 | 0.00 | (−0.02, 0.02) | 0.6827 |
| U/L | | | | | | | | | | | | |
| MSDC- | 20 | −0.02 | (−0.04, 0.01) | 0.2055 | 20 | −0.01 | (−0.03, 0.01) | 0.2995 | | | | |
| 0602K | | | | | | | | | | | | |
| Dose | | | | | | | | | | | | |

| | |
|---|---|
| Adjusted R²: 0.4185 | Adjusted R²: 0.4053 |
| Adjusted R² w/Dose: 0.4183 | |

Multivariable models for each outcome including predictors found prognostic for any are shown in Table 9.

TABLE 9
Multivariable modeling results for changes in histological parameters in the EMMINENCE trial

| | Ballooning | | | | Steatosis | | | | Inflammation |
|---|---|---|---|---|---|---|---|---|---|
| | Effect | | | | Effect | | | | Effect |
| | size for | Effect | | | size for | Effect | | | size for |
| | change | Size | | P- | change | Size | | P- | change |
| Parameter | of: | (95% CI) | | value | of: | (95% CI) | | value | of: |
| Baseline | | | | | | | | | |
| Variables | | | | | | | | | |
| Age, years | 57.00 vs. | 0.04 | (−0.03, 0.10) | 0.008 | 5 | −0.06 | (−0.11, 0.02) | 0.007 | 5 |
| | 50.00 | | | | | | | | |
| | 65.00 vs. | −0.04 | (−0.14, 0.06) | | | | | | |
| | 57.00 | | | | | | | | |
| Diabetes | Yes vs. | −0.05 | (−0.26, 0.16) | 0.642 | Yes vs. | 0.08 | (−0.13, 0.28) | 0.451 | Yes vs. |
| | No | | | | No | | | | No |
| Weight, kg | 5 | 0.00 | (−0.02, 0.02) | 0.857 | 99.06 vs. | 0.06 | (−0.01, 0.13) | 0.124 | 5 |
| | | | | | 85.91 | | | | |
| | | | | | 113.40 vs. | 0.00 | (−0.06, 0.07) | | |
| | | | | | 99.06 | | | | |
| AST, U/L | Doubling | 0.30 | (0.09, 0.50) | 0.005 | Doubling | 0.08 | (−0.01, 0.21) | 0.102 | Doubling |
| Triglyc- | Doubling | 0.09 | (−0.02, 0.21) | 0.117 | 1 | −0.02 | (−0.07, 0.03) | 0.426 | 1 |
| erides, | | | | | | | | | |
| mmol/L | | | | | | | | | |
| HbA1c, % | 0.25 | 0.04 | (0.01, 0.06) | 0.010 | 0.25 | 0.02 | (−0.00, 0.04) | 0.076 | 0.25 |
| GGT, U/L | 10 | 0.01 | (−0.01, 0.02) | 0.368 | 43.00 vs. | −0.01 | (−0.04, 0.03) | 0.884 | 10 |
| | | | | | 31.00 | | | | |
| | | | | | 71.00 vs. | −0.02 | (−0.09, 0.06) | | |
| | | | | | 43.00 | | | | |
| Insulin, | 5 | 0.02 | (0.01, 0.04) | 0.007 | 5 | −0.00 | (−0.02, 0.01) | 0.766 | 5 |
| uIU/mL | | | | | | | | | |
| ELF | 1 | 0.02 | (−0.09, 0.13) | 0.744 | 1 | 0.07 | (−0.03, 0.18) | 0.172 | 1 |
| CYFRA-18, | 100 | 0.02 | (−0.00, 0.04) | 0.094 | 100 | 0.01 | (−0.01, 0.03) | 0.571 | 100 |
| U/L | | | | | | | | | |
| Change from | | | | | | | | | |
| Baseline | | | | | | | | | |
| Variables | | | | | | | | | |
| Weight, kg | 5 | 0.02 | (−0.05, 0.09) | 0.563 | 5 | 0.08 | (0.01, 0.15) | 0.021 | 5 |
| AST, U/L | 5 | 0.02 | (−0.01, 0.05) | 0.099 | 5 | 0.02 | (−0.01, 0.05) | 0.113 | 5 |
| HbA1c, % | 0.25 | 0.03 | (0.01, 0.06) | 0.017 | 0.25 | 0.03 | (−0.00, 0.05) | 0.057 | 0.25 |
| GGT, U/L | 10 | 0.01 | (−0.02, 0.04) | 0.616 | 10 | 0.03 | (0.00, 0.06) | 0.034 | 10 |
| CYFRA-18, | 100 | 0.02 | (0.00, 0.04) | 0.025 | 100 | 0.01 | (−0.01, 0.02) | 0.473 | 100 |
| U/L | | | | | | | | | |

| | | |
|---|---|---|
| Adjusted R²: 0.3850 | Adjusted R²: 0.3390 | Adjusted R²: 0.4775 |

| | | | | Fibrosis | | | |
|---|---|---|---|---|---|---|---|
| | Inflammation | | | Effect | | | |
| | Effect | | | size for | Effect | | |
| | Size | | P- | change | Size | | P- |
| Parameter | (95% CI) | | value | of: | (95% CI) | | value |
| Baseline | | | | | | | |
| Variables | | | | | | | |
| Age, years | −0.04 | (−0.07, 0.01) | 0.006 | 5 | 0.03 | (−0.03, 0.08) | 0.348 |
| Diabetes | 0.05 | (−0.09, 0.18) | 0.502 | Yes vs. | 0.21 | (−0.04, 0.46) | 0.094 |
| | | | | No | | | |
| Weight, kg | −0.00 | (−0.02, 0.01) | 0.543 | 5 | 0.01 | (−0.02, 0.03) | 0.513 |
| AST, U/L | 0.11 | (−0.02, 0.24) | 0.094 | Doubling | 0.51 | (0.26, 0.76) | <.001 |
| Triglyc- | 0.01 | (−0.03, 0.04) | 0.666 | Spline | 0.19 | (0.00, 0.37) | 0.134 |
| erides, | | | | (<=2.48) | | | |
| mmol/L | | | | | | | |
| | | | | spline | −0.04 | (−0.12, 0.04) | |
| | | | | (>2.48) | | | |
| HbA1c, % | 0.00 | (−0.01, 0.02) | 0.620 | 0.25 | 0.00 | (−0.03, 0.03) | 0.856 |
| GGT, U/L | −0.00 | (−0.01, 0.01) | 0.816 | 10 | 0.01 | (−0.00, 0.03) | 0.161 |
| Insulin, | 0.01 | (0.00, 0.02) | 0.025 | 5 | 0.00 | (−0.02, 0.02) | 0.862 |
| uIU/mL | | | | | | | |
| ELF | 0.09 | (0.02, 0.16) | 0.015 | 1 | 0.14 | (0.01, 0.28) | 0.033 |
| CYFRA-18, | 0.02 | (0.01, 0.03) | 0.006 | spline | 0.05 | (0.00, 0.09) | 0.106 |
| U/L | | | | (<=1202) | | | |
| | | | | spline | −0.02 | (−0.06, 0.02) | |
| | | | | (>1202) | | | |
| Change from | | | | | | | |
| Baseline | | | | | | | |
| Variables | | | | | | | |
| Weight, kg | −0.01 | (−0.06, 0.04) | 0.692 | 5 | 0.03 | (−0.05, 0.12) | 0.478 |
| AST, U/L | 0.02 | (0.00, 0.04) | 0.025 | 5 | 0.08 | (0.04, 0.11) | <.001 |
| HbA1c, % | 0.03 | (0.01, 0.04) | 0.003 | 0.25 | 0.02 | (−0.02, 0.05) | 0.341 |
| GGT, U/L | −0.01 | (−0.02, 0.01) | 0.503 | 10 | −0.01 | (−0.04, 0.03) | 0.706 |
| CYFRA-18, | 0.01 | (−0.00, 0.02) | 0.082 | 100 | 0.00 | (−0.02, 0.02) | 0.683 |
| U/L | | | | | | | |

| | |
|---|---|
| Adjusted R²: 0.4775 | Adjusted R²: 0.4185 |

Generally, inclusion of the additional covariates, or addition of MSDC-0602K dose (data not shown) did not change parameter estimates for any given outcome. Each of the models that included 12-month changes in weight, AST, HbA1c, GGT, and CK-18, accounted for approximately 40% of the variance (i.e., adjusted R2≈0.4) in each of the biopsy score changes (Table 9). With multivariable adjustment, change in weight was significantly associated with changes in 1 of the 4 histologic features, AST with 2 (and nearly significant for the other 2), HbA1c with 2 (nearly significant for another one), GGT with one, and CK-18 with one (nearly significant for another one).

These same predictors accounted for approximately the same proportion of variance in 12-month change in NAS with an adjusted R2 of 0.41 (Table 10).

TABLE 10
Multivariable models for histological outcomes in the EMMINENCE trial

| | | | | | | | | | Improvement in | | | |
|---|---|---|---|---|---|---|---|---|---|---|---|---|
| | | | | | | | | | Fibrosis with No | | | |
| | NAS | | | | Resolution of NASH | | | | Worsening of NASH | | | |
| | Effect | | | | Effect | | | | Effect | | | |
| | size for | Effect | | | size for | Odds | | | size for | Odds | | |
| | change | Size | | P- | change | Ratio | | P- | change | Ratio | | P- |
| Parameter | of: | (95% CI) | | value | of: | (95% CI) | | value | of: | (95% CI) | | value |
| Baseline | | | | | | | | | | | | |
| Variables | | | | | | | | | | | | |
| Age, years | 57.00 vs. | −0.13 | (−0.25, −0.01) | <.001 | 5 | 1.00 | (0.85, 1.19) | 0.964 | 5 | 1.07 | (0.90, 1.26) | 0.445 |
| | 50.00 | | | | | | | | | | | |
| | 65.00 vs. | −0.34 | (−0.52, −0.16) | . | | | | | | | | |
| | 57.00 | | | | | | | | | | | |
| Diabetes | Yes vs. | −0.01 | (−0.39, 0.36) | 0.939 | Yes vs. | 1.10 | (0.54, 2.25) | 0.789 | Yes vs. | 0.89 | (0.45, 1.73) | 0.724 |
| | No | | | | No | | | | No | | | |
| Weight, kg | 5 | 0.01 | (−0.03, 0.05) | 0.626 | 5 | 1.02 | (0.94, 1.10) | 0.669 | 5 | 0.99 | (0.92, 1.07) | 0.877 |
| AST, U/L | Doubling | 0.36 | (−0.00, 0.73) | 0.054 | Doubling | 0.94 | (0.90, 0.97) | <.001 | 1 | 0.97 | (0.94, 1.00) | 0.068 |
| Triglyc- | spline | 0.27 | (−0.01, 0.55) | 0.172 | 1 | 0.90 | (0.70, 1.17) | 0.432 | spline | 0.60 | (0.36, 1.01) | 0.062 |
| erides, | (<=2.48) | | | | | | | | (<=2.48) | | | |
| mmol/L | | | | | | | | | | | | |
| | spline | −0.04 | (−0.16, 0.08) | . | | | | | spline | 1.29 | (0.99, 1.69) | . |
| | (>2.48) | | | | | | | | (>2.48) | | | |
| HbA1c, % | 0.25 | 0.05 | (−0.00, 0.10) | 0.060 | 0.25 | 0.94 | (0.84, 1.05) | 0.274 | 0.25 | 0.94 | (0.85, 1.04) | 0.217 |
| GGT, U/L | 10 | 0.00 | (−0.03, 0.03) | 0.980 | 10 | 0.89 | (0.78, 1.02) | 0.087 | 10 | 0.97 | (0.91, 1.05) | 0.483 |
| Insulin, | 5 | 0.03 | (−0.00, 0.06) | 0.092 | 5 | 0.95 | (0.86, 1.05) | 0.287 | 5 | 0.95 | (0.87, 1.05) | 0.337 |
| uIU/mL | | | | | | | | | | | | |
| ELF | 1 | 0.16 | (−0.04, 0.35) | 0.114 | 1 | 0.81 | (0.54, 1.22) | 0.310 | 1 | 1.06 | (0.73, 1.54) | 0.750 |
| CYFRA-18, | 100 | 0.05 | (0.01, 0.09) | 0.008 | 849.93 vs. | 0.84 | (0.61, 1.16) | 0.285 | 100 | 0.93 | (0.84, 1.03) | 0.187 |
| U/L | | | | | 569.74 | | | | | | | |
| | | | | | 1217.80 vs. | 0.91 | (0.66, 1.26) | | | | | |
| | | | | | 849.93 | | | | | | | |
| Change from | | | | | | | | | | | | |
| Baseline | | | | | | | | | | | | |
| Variables | | | | | | | | | | | | |
| Weight, kg | 5 | 0.11 | (−0.02, 0.24) | 0.099 | 5 | 0.93 | (0.73, 1.18) | 0.533 | 5 | 1.03 | (0.82, 1.28) | 0.825 |
| AST, U/L | 5 | 0.06 | (0.00, 0.11) | 0.040 | 5 | 0.85 | (0.70, 1.03) | 0.096 | 5 | 0.89 | (0.74, 1.07) | 0.210 |
| HbA1c, % | 0.25 | 0.08 | (0.03, 0.13) | 0.003 | 0.25 | 0.88 | (0.77, 1.01) | 0.080 | 0.25 | 0.94 | (0.84, 1.06) | 0.336 |
| GGT, U/L | 10 | 0.02 | (−0.03, 0.07) | 0.384 | 10 | 1.00 | (0.80, 1.25) | 0.996 | 10 | 0.97 | (0.84, 1.11) | 0.649 |
| CYFRA-18, | 100 | 0.04 | (0.01, 0.07) | 0.012 | 100 | 0.98 | (0.90, 1.05) | 0.513 | 100 | 0.92 | (0.82, 1.03) | 0.150 |
| U/L | | | | | | | | | | | | |
| MSDC- | 20 | −0.03 | (−0.07, 0.00) | 0.0552 | 20 | 1.07 | (0.99, 1.14) | 0.0734 | 20 | 1.00 | (0.94, 1.07) | 0.9637 |
| 0602K | | | | | | | | | | | | |
| Dose | | | | | | | | | | | | |

| | | |
|---|---|---|
| Adjusted R²: 0.4118 | C-Statistic: 0.8207 | C-Statistic: 0.7593 |
| Adjusted R² w/dose: 0.4170 | C-Statistic w/dose: 0.8274 | C-Statistic w/dose: 0.7593 |

After adjustment for covariates associated with change in any individual histologic feature, 12-month changes in AST, HbA1c, and CK-18[M65] were statistically significantly associated with 12-month change in NAS. Changes in AST and HbA1c were nearly significantly associated (p<0.10) with NASH resolution without worsening fibrosis, while change in AST was nearly statistically significantly associated (p<0.10) with fibrosis improvement without worsening of NASH (Table 10).

Canonical Correlation

The first canonical variate was found to be statistically significant (p<0.0001), with a canonical correlation of about 0.49 pointing towards a positive linear correlation between the two sets of changes (12-month changes from baseline in biomarkers and in biopsy results). Standardized canonical coefficients (the weights used to maximize the correlation) and correlations of each of the component variables with the canonical variate suggest that changes in AST, HbA1c, and CK-18[M65] had the strongest influence on the first canonical variate for the biomarker, while all four biopsy outcome variables appeared to contribute more or less equally to their canonical variate, with the strongest influence observed for changes in inflammation (Table 11).

TABLE 11
Characteristics of NASH clinical trials included in meta-analysis

| Study | | | | | | | | | | | |
|---|---|---|---|---|---|---|---|---|---|---|---|
| Clinical | | | Control | | Active 1 | | Active 2 | | Active 3 | | |
| Trial | | | | Sample | | Sample | | Sample | | Sample | Follow-up |
| Reference | Study | Phase | Control | Size | Treatment | Size | Treatment | Size | Treatment | Size | Period |
| Ratzui et al. | GOLDEN | 2b | Placebo | 92 | Elafibranor | 93 | Elafibranor | 89 | | | 12- |
| | | | | | 80 mg | | 120 mg | | | | months |
| Neuschwander- | FLINT | 2b | Placebo | 141 | Obeticholic | 142 | | | | | 17- |
| Tetri, et al. | | | | | acid 25 mg | | | | | | months |
| Younossi, | REGENERATE | 3 | Placebo | 311 | Obeticholic | 312 | Obeticholic | 308 | | | 18- |
| et al. | | | | | acid | | acid | | | | months |
| | | | | | 10 mg + | | 25 mg + | | | | |
| | | | | | statin | | statin | | | | |
| Sanyal, et al. | PIVENS | Academic | Placebo | 83 | Pioglitazone | 80 | Vitamin | 84 | | | 22- |
| | | | | | 30 mg | | E | | | | months |
| Webber, et al. | WEBBER | Academic | Placebo | 37 | Pioglitazone | 37 | | | | | 12- |
| | | | | | 30 mg | | | | | | months |
| Belfort, et al. | BELFORT | Academic | Placebo | 21 | Pioglitazone | 26 | | | | | 6- |
| | | | | | 45 mg | | | | | | months |
| Cusi, et al. | CUSI | Academic | Placebo | 51 | Pioglitazone | 50 | | | | | 18- |
| | | | | | 45 mg | | | | | | months |
| Ratziu, et al. | FLIRT | Academic | Placebo | 31 | Rosiglitazone | 32 | | | | | 12- |
| | | | | | 8 mg | | | | | | months |
| Armstrong, | LEAN | Academic | Placebo | 23 | Liraglutide | 22 | | | | | 11- |
| et al. | | | | | 1.8 mg | | | | | | months |
| Friedman, | CENTAUR | 2b | Placebo | 144 | Cenicriviroc | 145 | | | | | 12- |
| et al. | | | | | 150 mg | | | | | | months |
| Harrison, | Madrigal | 2b | Placebo | 38 | Resmetirom | 78 | | | | | 9- |
| et al. | | | | | 80 mg | | | | | | months |
| Presentation, | ARREST | 2b | Placebo | 48 | Aramchol | 101 | Aramchol | 98 | | | 12- |
| AASLD 2018 | | | | | 400 mg | | 600 mg | | | | months |
| Harrison, | Emminence | 2b | Placebo | 94 | MSDC- | 99 | MSDC- | 98 | MSDC- | 101 | 12- |
| et al. | | | | | 0602L | | 0602K | | 0602K | | months |
| | | | | | 62.5 mg | | 125 mg | | 250 mg | | |
| Harrison, | ENCORE- | 2b | Placebo | 105 | Emricasan | 107 | Emricasan | 106 | | | 72- |
| et al. | NF | | | | 5 mg | | 50 mg | | | | weeks |
| Georgescu, | GEORGESCU | | | | Valsartan | 26 | Telmisartan | 28 | | | 20- |
| et al. | | | | | 80 mg | | 20 mg | | | | months |
| Nogueira, | NOGUEIRA | | Placebo | 23 | Omega-3 | 27 | | | | | 6- |
| et al. | | | | | 1 g | | | | | | months |
| Lueschner, | LEUSCHNER | | Placebo | 91 | UDCA | 95 | | | | | 18- |
| et al. | | | | | 23-28 | | | | | | months |
| | | | | | mg/kg/day | | | | | | |

AST, HbA1c, and CK-18 also had the highest correlations with the biopsy canonical variate.

Meta-Regressions

Seventeen NASH clinical trials, comprising 3,717 patients, were included in the meta-analyses (FIG. 6). Study characteristics are described in Table 12.

TABLE 12
Change from Baseline in LFTs and Biomarkers by Study Arm

| | | % Change from Baseline | | | | | | | |
|---|---|---|---|---|---|---|---|---|---|
| | | | | | | | | Alk | |
| Trial | Treatment | ALT | AST | CK-18 | HbA1c | GGT | Insulin | Phos | ELF |
| GOLDEN | Placebo | −4.86 | −6.97 | | | −15.36 | | 4.34 | |
| | Elafibranor | −9.72 | −16.87 | | | −34.54 | | −25.87 | |
| | 80 mg | | | | | | | | |
| | Elafibranor | −19.75 | −30.22 | | | −41.68 | | −26.41 | |
| | 120 mg | | | | | | | | |
| FLINT | Placebo | −21.95 | −17.03 | | 0.62 | −7.89 | 7.25 | −7.41 | |
| | Obeticholic | −45.78 | −41.83 | | 0.76 | −47.44 | 14.43 | 14.63 | |
| | acid 25 mg | | | | | | | | |
| REGENERATE | Placebo + | −19.60 | −16.64 | | 0.00 | −16.04 | | 14.99 | |
| | statin | | | | | | | | |
| | Obeticholic | −31.48 | −24.91 | | 4.15 | −21.95 | | 7.10 | |
| | acid | | | | | | | | |
| | 10 mg + | | | | | | | | |
| | statin | | | | | | | | |
| | Obeticholic | −44.89 | −35.79 | | 4.31 | −47.70 | | −5.04 | |
| | acid | | | | | | | | |
| | 25 mg + | | | | | | | | |
| | statin | | | | | | | | |
| PIVENS | Placebo | −24.81 | −6.91 | | | −5.80 | 7.83 | −4.63 | |
| | Pioglitazone | −49.76 | −37.78 | | | −35.17 | −9.52 | −13.95 | |
| | 30 mg | | | | | | | | |
| | Vitamin E | −43.02 | −36.10 | | | −25.00 | 2.73 | −12.08 | |
| WEBBER | Placebo | −8.20 | | | 1.69 | −4.69 | −10.80 | | |
| | Pioglitazone | −40.28 | | | −3.45 | −56.34 | 22.75 | | |
| | 30 mg | | | | | | | | |
| BELFORT | Placebo | −34.43 | −21.43 | | −1.61 | | 0.00 | | |
| | Pioglitazone | −58.21 | −40.43 | | −11.29 | | −33.33 | | |
| | 45 mg | | | | | | | | |
| CUSI | Placebo | −22.81 | −11.63 | −8.45 | −1.90 | | 6.25 | | |
| | Pioglitazone | −56.45 | −38.30 | −53.68 | −7.81 | | −41.18 | | |
| | 45 mg | | | | | | | | |
| FLIRT | Placebo | −10.00 | | | 3.04 | | | | |
| | Rosiglitazone | −30.00 | | | −3.21 | | | | |
| | 8 mg | | | | | | | | |
| LEAN | Placebo | −15.45 | −16.86 | −26.14 | 0.00 | −6.26 | −13.50 | −1.38 | −1.06 |
| | Liraglutide | −34.55 | −30.98 | −46.95 | −8.98 | −37.03 | −9.58 | −6.71 | −3.23 |
| | 1.8 mg | | | | | | | | |
| CENTAUR | Placebo | 1.17 | 5.83 | −22.02 | 1.26 | 22.66 | 13.05 | 19.06 | |
| | Cenicriviroc | 11.23 | 11.67 | −12.34 | −0.75 | 2.68 | 2.14 | 1.24 | |
| | 150 mg | | | | | | | | |
| MADRIGAL | Placebo | 18.30 | 9.47 | −13.68 | 2.49 | 72.54 | | 12.48 | −1.96 |
| | Resmetirom | −30.80 | −21.08 | −35.15 | 0.63 | −18.76 | | 7.85 | −7.17 |
| | 80 mg | | | | | | | | |
| ARREST | Placebo | 17.58 | | | 4.92 | | | | |
| | Aramchol | −18.06 | | | −0.62 | | | | |
| | 400 mg | | | | | | | | |
| | Aramchol | −32.02 | | | −1.79 | | | | |
| | 600 mg | | | | | | | | |
| EMMINENCE | Placebo | −11.85 | −4.38 | 2.33 | 3.98 | 6.46 | 14.74 | 1.47 | 0.60 |
| | MSDC- | −15.71 | −8.08 | −7.08 | −4.16 | −17.56 | −10.85 | −6.28 | 1.16 |
| | 0602K | | | | | | | | |
| | 62.5 mg | | | | | | | | |
| | MSDC- | −28.13 | −21.90 | −22.13 | −3.79 | −13.47 | −30.08 | −10.70 | 0.09 |
| | 0602K | | | | | | | | |
| | 125 mg | | | | | | | | |
| | MSDC- | −23.82 | −11.81 | −8.61 | −3.89 | −28.14 | −30.62 | −18.74 | 0.89 |
| | 0602K | | | | | | | | |
| | 250 mg | | | | | | | | |
| ENCORE-HF | Placebo | −20.54 | −20.21 | −17.91 | | | | | |
| | Emricasan | −35.28 | −18.47 | −36.86 | | | | | |
| | 5 mg | | | | | | | | |
| | Emricasan | −45.17 | −19.83 | −45.07 | | | | | |
| | 50 mg | | | | | | | | |
| GEORGESCU | Valsartan | −23.22 | −18.82 | | | −20.60 | | | |
| | 80 mg | | | | | | | | |
| | Telmisartan | −30.29 | −34.64 | | | −2.83 | | | |
| | 20 mg | | | | | | | | |
| LEUSCHNER | Placebo | −34.84 | −23.45 | | | −18.53 | | −5.45087 | |
| | UDCA | −40.55 | −27.95 | | | −59.94 | | −6.37576 | |
| | 23-28 | | | | | | | | |
| | mg/kg/day | | | | | | | | |

Total sample sizes ranged from 47 to 931 patients and follow-up from 6 to 22 months. Laboratory changes from baseline and changes in histological outcomes for each study are given in Table 13.

TABLE 13
Change from baseline in histological components endpoint counts

| | | | | | | Endpoints, n/N (%) | |
|---|---|---|---|---|---|---|---|
| | | Change from Baseline, mean | | | | NASH | Fibrosis |
| Trial | Treatment | Steatosis | Ballooning | Inflammation | Fibrosis | Resolution | Improvement |
| GOLDEN | Placebo | | | | | 11/92 | |
| | Elafibranor | | | | | 12/93 | |
| | 80 mg | | | | | | |
| | Elafibranor | 0.02 | −0.07 | −0.32 | −0.07 | 17/89 | |
| | 120 mg | | | | | | |
| FLINT | Placebo | −0.41 | −0.15 | −0.15 | 0.13 | | 19/98 |
| | Obeticholic | −0.78 | −0.46 | −0.48 | −0.20 | | 36/102 |
| | acid 25 mg | | | | | | |
| REGENERATE | Placebo + | | | | | 25/311 | 37/311 |
| | statin | | | | | | |
| | Obeticholic | | | | | 35/312 | 55/312 |
| | acid | | | | | | |
| | 10 mg + | | | | | | |
| | statin | | | | | | |
| | Obeticholic | | | | | 36/308 | 71/308 |
| | acid | | | | | | |
| | 25 mg + | | | | | | |
| | statin | | | | | | |
| PIVENS | Placebo | −0.10 | −0.10 | −0.20 | −0.10 | 17/83 | |
| | Pioglitazone | −0.80 | −0.40 | −0.70 | −0.40 | 38/80 | |
| | 30 mg | | | | | | |
| | Vitamin E | −0.70 | −0.30 | −0.60 | −0.30 | 30/84 | |
| WEBBER | Placebo | −0.33 | −0.03 | −0.13 | −0.03 | | |
| | Pioglitazone | −0.65 | −0.45 | −0.32 | −0.45 | | |
| | 30 mg | | | | | | |
| BELFORT | Placebo | −0.28 | −0.10 | −0.28 | −0.10 | | |
| | Pioglitazone | −1.08 | −0.61 | −0.81 | −0.61 | | |
| | 45 mg | | | | | | |
| CUSI | Placebo | −0.18 | 0.00 | −0.10 | 0.00 | 9/51 | |
| | Pioglitazone | −1.10 | −0.50 | −0.60 | −0.50 | 23/50 | |
| | 45 mg | | | | | | |
| FLIRT | Placebo | | 0.23 | −0.13 | −0.18 | | |
| | Rosiglitazone | | 0.13 | −0.09 | 0.03 | | |
| | 8 mg | | | | | | |
| LEAN | Placebo | −0.32 | −0.20 | −0.20 | 0.18 | 2/26 | |
| | Liraglutide | −0.52 | −0.50 | −0.20 | −0.17 | 9/26 | |
| | 1.8 mg | | | | | | |
| CENTAUR | Placebo | | | | | | 15/244 |
| | Cenicriviroc | | | | | | 29/145 |
| | 150 mg | | | | | | |
| MADRIGAL | Placebo | | | | | 5/34 | 8/34 |
| | Resmetirom | | | | | 18/73 | 21/73 |
| | 80 mg | | | | | | |
| ARREST | Placebo | | | | | 2/40 | 7/40 |
| | Aramchol | | | | | 6/80 | 17/80 |
| | 400 mg | | | | | | |
| | Aramchol | | | | | 13/78 | 23/78 |
| | 600 mg | | | | | | |
| EMMINENCE | Placebo | −0.36 | −0.18 | −0.05 | 0.03 | 15/94 | 16/94 |
| | MSDC- | −0.57 | −0.36 | −0.08 | 0.06 | 17/99 | 20/99 |
| | 0602K | | | | | | |
| | 62.5 mg | | | | | | |
| | MSDC- | −0.51 | −0.42 | −0.22 | −0.12 | 23/98 | 23/98 |
| | 0602K | | | | | | |
| | 125 mg | | | | | | |
| | MSDC- | −0.67 | −0.38 | −0.12 | −0.06 | 27/101 | 25/101 |
| | 0602K | | | | | | |
| | 250 mg | | | | | | |
| ENCORE-HF | Placebo | −0.08 | −0.22 | −0.12 | 0.06 | 11/105 | 20/105 |
| | Emricasan | −0.20 | 0.04 | −0.11 | 0.41 | 4/107 | 12/107 |
| | 5 mg | | | | | | |
| | Emricasan | −0.27 | 0.13 | 0.09 | 0.36 | 7/106 | 13/106 |
| | 50 mg | | | | | | |
| GEORGESCU | Valsartan | −0.34 | −0.11 | 0.08 | −0.31 | | |
| | 80 mg | | | | | | |
| | Telmisartan | −0.50 | −0.58 | −0.36 | −0.75 | | |
| | 20 mg | | | | | | |
| NOGUEIRA | Placebo | 0.20 | −0.20 | −0.20 | 0.10 | | |
| | Omega-3 | −0.10 | −0.10 | 0.20 | 0.10 | | |
| | 1 g | | | | | | |
| LEUSCHNER | Placebo | −0.57 | −0.26 | −0.19 | | | |
| | UDCA | −0.55 | −0.16 | −0.51 | | | |
| | 23-28 | | | | | | |
| | mg/kg/day | | | | | | |

Varying degrees of association of biomarker changes with changes in biopsy findings were identified. A decrease in insulin was associated with a decrease in steatosis (FIG. 11), while a decrease in GGT was associated with a decrease in inflammation (FIG. 15). Decreases in AST and HbA1c were associated with decreases in ballooning, inflammation, steatosis, and fibrosis while a decrease in CK-18 was associated with a decrease in ballooning, inflammation, and fibrosis, though not steatosis (FIG. 1). Results for ALT followed those of AST. Other biomarker responses were not associated with histologic score changes.

Greater treatment effects (i.e., lower treatment ratios) on AST were associated with greater treatment effects (i.e., lower mean treatment differences in histology scores) on ballooning, steatosis, inflammation, and fibrosis (FIG. 2). Treatment effects on HbA1c were not associated with treatment effects on any of these histology scores, while greater treatment effects on CK-18 were associated with greater treatment effects on steatosis, inflammation, and fibrosis but not with ballooning.

Changes in AST and CK-18, but not changes in HbA1c were associated with changes in NAS (FIG. 3). Changes in AST and HbA1c but not CK-18 were associated with the rate of NASH resolution. None of these three biomarkers was associated with fibrosis improvement without worsening of NASH, although the change in AST was nearly so. Only the treatment effect on AST was associated with treatment effect on NASH resolution without worsening of fibrosis, and only CK-18 with fibrosis improvement without worsening of NASH (FIG. 4).

Composite Endpoint

Based on the meta-regression results, the three biomarkers best associated with liver histology improvements overall were AST, CK-18 and HbA1c with average ranks across all outcomes of 6.00, 4.67, and 4.21, respectively. The weighted average (0.403xAST, 0.314axCK-18, 0.283xHbA1c) of changes in these three biomarkers may be considered reflective of expected overall effects on histologic parameters. The five interventions with the largest treatment effects on this new surrogate endpoint were pioglitazone, Aramachol, resmetiron MSDC-0602K, and liraglutide. In two studies [ARREST (Aramchol) and Madrigal phase 2b (resmetiron)], treatment effects relative to placebo were driven primarily by large adverse changes in the placebo group, while in other studies such as the pioglitazones studies of Cusi and Belfort, the LEAN study of liraglutide, ENCORE-NF (Emricasan) and FLINT (obeticholic acid) large improvements in the placebo group were observed, suggesting an overall trend to improvement in all patients enrolled in the study.

In the pooled treatment groups in EMMINENCE, the AUROC for the baseline-adjusted 12-month change in the AST/HbA1c/CK-18 composite was 0.7553 for fibrosis improvement without worsening of NASH and 0.7880 for NASH resolution without worsening of fibrosis. The measure with the highest AUROCs among the others examined was Fibroscan® stiffness for both fibrosis improvement without worsening of NASH (0.6679) and for NASH resolution without worsening of fibrosis (0.6627).

Discussion

The results of the present analysis suggest that in patients with NASH treated with new interventions, a weighted score of AST, CK-18 and HbA1c changes is associated with histologic improvement in NASH. This result has been seen in both analysis of the EMMINENCE study and a meta-analysis of 17 studies including 3,717 patients.

NASH is a substantial disorder with high prevalence leading to significant adverse outcomes including cirrhosis, hepatocellular carcinoma and need for liver transplantation. Its combined high prevalence and adverse effects have therefore very important consequences on the health of patients as well as significant economic effects on health systems. Therefore, developing new therapies for NASH is of utmost importance. However, such development of new interventions for NASH has been hindered by the need to perform complex and expensive studies with “paired” liver biopsies, i.e., liver biopsies before and after 1-1.5 years' treatment, for the initial assessment of the efficacy of new treatments. Beyond being very complex to perform and expensive, “paired” liver biopsies studies are fraught with significant problems. First, liver biopsies have some risks leading to adverse events. Second, they are limited by sampling errors, as NASH is not uniformly present at the same severity throughout the liver. Finally, in a post hoc analysis of the EMMINENCE study we have found that liver biopsy interpretation is fraught with inter-reader variability. Different expert hepatopathologists frequently read the same biopsies differently leading to inability to accurately detect changes in NASH status, introducing substantial noise into the assessment of new intervention for NASH.

As of now, no drug or intervention has been approved for the treatment of NASH. Some of this lack of progress may relate to the complexity, cost and lack of accuracy of “paired” biopsies studies. Hence, in the current analysis we sought to identify simple, noninvasive measures to assess NASH improvements. Our analysis in both the EMMINENCE study (using either multivariate regression or canonical models) and in a meta-analysis of 17 studies, changes in AST, CK-18 and HbA1c are best associated with overall histological improvements in NASH.

The weighted average of standardized changes in AST, CK-18, and HbA1c was used to assess the changes in the placebo and active arms of the studies included in the meta-analysis. Results presented in Table S10 suggest that many of these interventions are effective in improving this combined score, and hence potentially beneficial in NASH. Some of the interpretation is limited by unstable placebo effects—in some studies the placebo patients improved substantially suggesting that other beneficial interventions may have been implemented in parallel to the study drug during the follow up period, while in other studies, the placebo patients showed substantial worsening, suggesting that some of the suggested treatment effects may have been related to worsening in the placebo arm. This in turn, in smaller studies, may have been due to chance. However, the use of the proposed score enables better comparative qualitative assessment of treatment effects and can therefore facilitate a simple tool to assess efficacy of therapies in early NASH studies, avoiding the complexity and limitations of “paired” liver biopsy studies.

The baseline-adjusted change in the AST/HbA1c/CK-18 composite score better discriminated between histological responders and non-responders in patients in the EMMINENCE trial than did changes in other biochemical and imaging markers, even for fibrosis improvement without worsening of NASH. This finding may not be surprising as these other measures were specifically developed to detect fibrosis and not change in NASH over time.

Conclusions

Changes in AST, HbA1c, and CK-18 were identified in both covariate-adjusted models of the EMMINENCE study and meta-analysis of 17 studies including 3,717 patients to be associated with histological improvement in NASH. A weighted average of the standardized changes in these biomarkers can be used for assessment of the efficacy of new interventions for NASH in early development and hence reduce the dependence of these studies on complex, expensive, and unreliable “paired” liver biopsies.

1. A method of treating non-alcoholic fatty liver disease (NAFLD) and non alcoholic steatohepatitis (NASH) and/or metabolic syndrome, comprising administering to a subject in need thereof:
an initial dose of about 250 mg or about 125 mg of a compound of structural Formula (I): 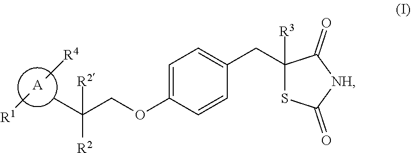
or a pharmaceutically acceptable salt thereof, for a period of time followed by administration in a dose less than the initial dose, wherein:
R¹ is hydrogen, halogen, substituted or unsubstituted alkyl, or —OR^{1A};
R² is halogen, hydroxyl, or optionally substituted aliphatic;
R^{2′} is hydrogen, or R² and R^{2′} may optionally be joined to form oxo;
R³ is hydrogen or deuterium;
R⁴ is hydrogen, halogen, substituted or unsubstituted alkyl, or —OR^{4A};
A is phenyl; and
R^{1A} and R^{4A} are independently hydrogen, halogen, —CF₃, —CCl₃, —CBr₃, —CI₃, —CHF₂, —CHCl₂, —CHBr₂, —CHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

2. The method of claim 1, wherein R³ is hydrogen.

3. The method of claim 2, wherein R⁴ is:
hydrogen, methyl, or —OR^{4A}; and
R^{4A} is methyl, ethyl, isopropyl, —CHF₂, or —CF₃.

4. The method of claim 3, wherein R⁴ is hydrogen.

5. The method of claim 1, wherein R′ is:
hydrogen, halogen, or —OR^{1A}; and
R^{1A} is substituted or unsubstituted alkyl.

6. (canceled)

7. (canceled)

8. The method of claim 5, wherein R¹ is —OR^{1A} and R^{1A} is substituted or unsubstituted alkyl.

9. (canceled)

10. (canceled)

11. (canceled)

12. (canceled)

13. (canceled)

14. (canceled)

15. (canceled)

16. (canceled)

17. (canceled)

18. The method of claim 1, wherein R² and R^{2′} are joined to form oxo.

19. The method of claim 1, wherein the compound of Formula (I) is: 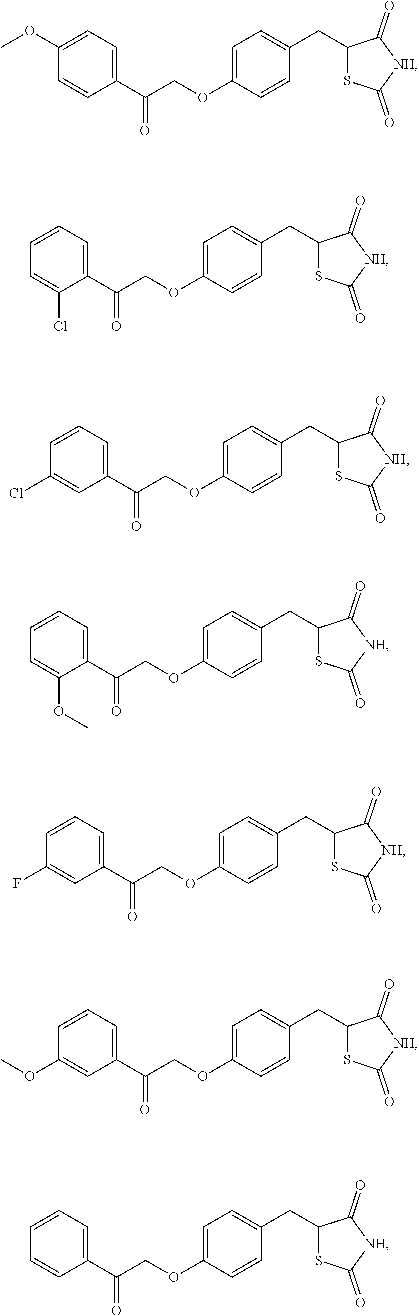 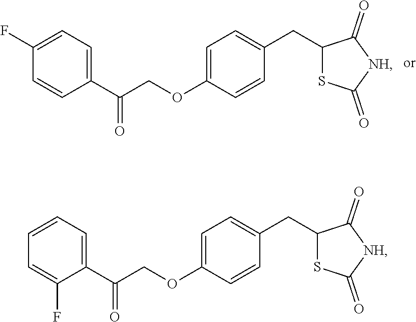 [SMILES: O=C1NC(=O)C(Cc2ccc(OCC(=O)c3ccc(F)cc3)cc2)S1 ; "or" ; O=C1NC(=O)C(Cc2ccc(OCC(=O)c3ccccc3F)cc2)S1]
or a pharmaceutically acceptable salt thereof.

20. The method of claim 1, wherein the compound of Formula (I) is: 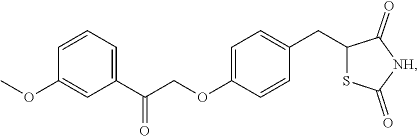 [SMILES: COc1cccc(C(=O)COc2ccc(CC3SC(=O)NC3=O)cc2)c1]
or a pharmaceutically acceptable salt thereof.

21. The method of claim 1, wherein the compound of Formula (I), or a pharmaceutically acceptable salt thereof, is administered orally.

22-195. (canceled)

196. The method of claim 21, wherein the pharmaceutically acceptable salt is a potassium salt.

197. The method of claim 1, wherein the initial dose is about 250 mg.

198. The method of claim 197, wherein the dose less than the initial dose is about 125 mg.

199. The method of claim 197, wherein the dose less than the initial dose is about 62.5 mg.

200. The method of claim 1, wherein the initial dose is about 125 mg.

201. The method of claim 200, wherein the dose less than the initial dose is about 62.5 mg.

202. The method of claim 1, wherein the period of time comprises one month.

203. The method of claim 202, wherein the period of time comprises three months.

204. The method of claim 1, wherein the period of time comprises six months.

205. The method of claim 1, wherein the period of time comprises twelve months.
